(19) United States
(12) Patent Application Publication
(10) Pub. No.: US 2021/0400985 A1
(43) Pub. Date: Dec. 30, 2021

(54) PLANT GROWTH-PROMOTING MICROBES, COMPOSITIONS, AND USES

(71) Applicant: PIONEER HI-BRED INTERNATIONAL, INC., JOHNSTON, IA (US)

(72) Inventors: RICHARD M BROGLIE, DES MOINES, IA (US); CAROLINE KOSTECKI, SAN RAFAEL, CA (US); HONOR RENEE LAFITTE, DAVIS, CA (US); ULRIKA LIDSTROM, SAN FRANCISCO, CA (US); STEVEN R PASZKIEWICZ, JOHNSTON, IA (US); JEFFREY R SCHUSSLER, MARION, IA (US); LAWRENCE KENT WOOD, KENSINGTON, CA (US)

(73) Assignee: PIONEER HI-BRED INTERNATIONAL, INC., JOHNSTON, IA (US)

(21) Appl. No.: 17/281,473

(22) PCT Filed: Oct. 9, 2019
(86) PCT No.: PCT/US19/55302

§ 371 Date: Mar. 30, 2021

Related U.S. Application Data
(60) Provisional application No. 62/743,722, filed on Oct. 10, 2018.

(51) Int. Cl. A01N 63/28; A01N 63/60; C12N 1/20; A01H 6/46; A01H 5/10

(52) U.S. Cl. CPC A01N 63/28; A01N 63/60; C12R 2001/06; A01H 6/4684; A01H 5/10; C12N 1/205

(57) ABSTRACT

The present application relates to plant growth promoting microbes (PGPMs), compositions comprising these PGPMs and methods of using these PGPMs and a plant or plant seed, wherein the plant or plant seed comprises at least one grain yield enhancing trait, for enhancing plant health, plant growth and/or plant yield, and/or for preventing, inhibiting, or treating the development of plant pathogens or the development of phytopathogenic diseases.

### FIELD

The present application relates to plant growth promoting microbes (PGPMs), compositions comprising these PGPMs and methods of using these PGPMs and a plant or plant seed, wherein the plant or plant seed comprises at least one grain yield enhancing trait, for enhancing plant health, plant growth and/or plant yield, and/or for preventing, inhibiting, or treating the development of plant pathogens or the development of phytopathogenic diseases.

### REFERENCE TO A SEQUENCE LISTING SUBMITTED AS A TEXT FILE VIA EFS-WEB

The Sequence Listing created on Sep. 28, 2018 as a text file named “7788_Seq_List.txt,” and having a size of 269 kilobytes is hereby incorporated by reference pursuant to 37 C.F.R. § 1.52(e)(5).

### BACKGROUND

Plant growth promoting microbes (PGPMs), such as plant growth-promoting rhizobacteria (PGPR), have gained worldwide importance and acceptance for agricultural benefits. PGPMs can affect plant growth by different direct and indirect mechanisms. There is a considerable amount of ongoing scientific research directed to understanding PGPMs, including the aspects of their adaptation, effects on plant physiology and growth, induced systemic resistance, biocontrol of plant pathogens, bio-fertilization, viability of co-inoculation, interactions with plant microorganisms, and mechanisms of root colonization.

By virtue of their rapid rhizosphere colonization and stimulation of plant growth and/or yield, there is currently considerable interest in exploiting PGPMs to improve crop production and grain yield. In fact, the inoculation of cultivated plants with PGPMs is currently considered a promising agricultural approach. As environmental concerns increase, e.g., concerns about groundwater quality with excess fertilizer and pesticide exposure in foods, biological alternatives are promising and becoming necessary. Thus, developing biological treatments compatible with fertilizers and pesticides and/or even reducing the amount of these chemical compounds used could be a significant advancement in the agricultural industry.

There is a continuing and pressing need for the identification of new PGPMs, PGPM synthetic consortia, and/or testing of their compatibility with existing commercially available crop management products.

### SUMMARY

The embodiments of this application address the aforementioned need by providing new plant growth promoting microbes (PGPMs), isolates, cultures, compositions, synthetic consortia, and methods useful for enhancing the health, growth and/or yield of a plant, wherein the plant comprises at least one genetically modified or transgenic grain yield enhancing trait. Also provided are methods for the treatment of plants or plant seeds by using the microbial strains (PGPMs), isolates, cultures or compositions disclosed herein, wherein the plants or plant seeds comprise at least one modified grain yield enhancing trait. In some embodiments, the at least one modified grain yield enhancing trait comprises an increased expression of an endogenous polynucleotide encoding an endogenous polypeptide. Such traits can be introduced by breeding with plants containing other recombinant events or with plants containing native variations or genome edited variations. In some embodiments, the at least one modified or transgenic yield enhancing trait comprises a Maize MADS box ZmM28 gene. In another embodiment, the Maize MADS box ZmM28 gene encodes a polypeptide having at least 95% sequence identity to SEQ ID NO: 476. In some embodiments, the Maize MADS box ZmM28 comprises a polynucleotide sequence having at least 95% sequence identity to SEQ ID NO: 475. In some embodiments, the plant or plant seed is a maize plant or maize plant seed. In some embodiments, the plant or plant seed comprises event DP202216-6 (ATCC Accession No. PTA-124653; further described in Example 4 of U.S. application No. 62/659,579, herein incorporated by reference).

In some embodiments, the grain yield is at least about three bushels/acre when compared to the control maize plant, wherein the plant and the control plant are grown in a field under normal crop growing conditions. In some embodiments, the grain yield in the field range from about 2 to about 8 bu/acre when compared to the control population of maize plants grown in a population density of about 20,000 to about 50,000 plants per acre.

This application also provides non-naturally occurring plant varieties that are artificially infected with at least one microbial strain disclosed herein. Other embodiments provide seed, reproductive tissue, vegetative tissue, regenerative tissues, plant parts, or progeny of the non-naturally occurring plant varieties. Other embodiments further provide a method for preparing agricultural compositions.

Other embodiments provide isolated microbial strains (PGPMs), isolated cultures thereof, biologically pure cultures thereof, and enriched cultures thereof. In some embodiments, the microbial strain comprises a 16S rRNA gene comprising a nucleotide sequence having at least 97% sequence identity to any one of SEQ ID NOs.: 1-474. In some embodiments, the microbial strain comprises a 16S rRNA gene comprising a nucleotide sequence having at least 97% sequence identity to any one of SEQ ID NOs.: 165-474. In some embodiments, the microbial strain comprises a 16S rRNA gene comprising a nucleotide sequence having at least 97% sequence identity to any one of SEQ ID NOs.: 172-182. In some embodiments, a 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to any one of the nucleotide sequences as set forth in any one of the SEQ ID NOs.: 1-474. Some embodiments provide a genus of microorganisms comprising any of the DNA sequences described above and which enhances the health, growth and/or yield of a plant, as described herein. In some embodiments, the microbial strain is a P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445) S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived from any one of these strains.

Another embodiment provides a microbial composition that comprises a microbial strain, such as a microbial strain selected from those described herein, or a culture thereof and a plant or plant seed, wherein the plant or plant seed comprises at least one modified or transgenic grain yield enhancing trait. In some embodiments, the microbial composition comprises a microbial strain, wherein the 16S rRNA gene of said strain comprises a sequence selected from the group consisting of SEQ ID NOs.: 1-474. In some embodiments, the microbial composition comprises a microbial strain, wherein the 16S rRNA gene of said strain comprises a sequence selected from the group consisting of SEQ ID NOs.: 165-474, or a culture thereof. In some embodiments, the microbial composition comprises a microbial strain, wherein the 16S rRNA gene of said strain comprises a sequence selected from the group consisting of SEQ ID NOs.: 172-182, or a culture thereof. Any of the above microbial compositions may optionally further comprise a second microbial strain whose 16S rRNA gene sequence comprises a sequence selected from the group consisting of SEQ ID NOs.: 1-474, or a culture thereof.

In some embodiments, the microbial composition comprises one or more microbial strains selected from S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof and a plant or plant seed, wherein the plant or plant seed comprises at least one modified or transgenic grain yield enhancing trait.

Other embodiments provide a composition comprising a synthetic microbial consortium and a plant or plant seed, wherein the plant or plant seed comprises at least one modified or transgenic grain yield enhancing trait. In some embodiments, a synthetic consortium comprises a) a first set of microbes comprising one or more microbes that promote plant health, growth, and/or yield; and b) a second set of microbes comprising one or more microbes that increase the competitive fitness of the first set of microbes in a); wherein the first and the second sets of microbes are combined into a single mixture as a synthetic consortium. In some embodiments, the synthetic consortium or a composition promotes or enhances plant health, growth and/or yield. In some embodiments, the synthetic consortium or a composition thereof according to the present application is applied to a plant (or a part thereof), a seed, or a seedling.

In some embodiments, a microbial composition as described herein, such as any of the microbial compositions described above and below, further comprises an agriculturally effective amount of a compound or composition selected from, but not limited to, a nutrient, a fertilizer, an acaricide, a bactericide, a fungicide, an insecticide, a microbicide, a nematicide, and a pesticide and combinations thereof. In some embodiments of the microbial compositions described herein, the microbial composition further comprises a carrier, such as (but not limited to) an organic or an inorganic carrier and combinations thereof. In some embodiments, the carriers suitable for the microbial compositions include, but are not limited to, silt, peat, turf, talc, lignite, kaolinite, pyrophyllite, zeolite, montmorillonite, alginate, press mud, sawdust and vermiculite and combinations thereof. In some embodiments, the carrier is a plant seed. In some embodiments, the microbial composition is prepared as a formulation selected from, but not limited to, an emulsion, a colloid, a dust, a granule, a pellet, a powder, a spray, and a solution. In some embodiments, the microbial composition described herein is a seed coating formulation.

Other embodiments provide a plant seed treatment having a coating comprising a microbial strain or a culture thereof as described herein, wherein the plant seed comprises at least one modified or transgenic grain yield enhancing trait. Also provided is a plant or a seed having a coating comprising a microbial composition as described herein, wherein the plant or seed comprises at least one modified or transgenic grain yield enhancing trait.

Other embodiments provide a method for treating plant seeds or seed priming. In some embodiments, the method includes exposing or contacting the plant seed with a microbial strain according to the present embodiments or a culture thereof. In some embodiments, the method includes exposing or contacting the plant seed with a microbial composition according to the present embodiments.

Other embodiments provide a method for enhancing the health, growth and/or yield of a plant. In some embodiments, such method involves applying an effective amount of a microbial strain, or a culture thereof to the plant, a plant part, or to the plant's surroundings, wherein the plant seed comprises at least one modified or transgenic grain yield enhancing trait. In some embodiments, the method involves growing one or more microbial strains in a growth medium or soil of a host plant or plant part prior to or concurrent with the host plant's growth in said growth medium or soil. In some embodiments of the above method, a microbial strain is applied to the plant, plant part, or to the plant's surroundings (e.g., immediate soil layer or rhizosphere) in a culture or a composition according to the present embodiments at a concentration that is at least 2×, 5×, 10×, 100×, 500×, or 1000× the concentration of the same microbial strain found in nature or detected in an untreated control plant, plant part, or the control plant's surroundings, respectively. In some embodiments, upon or after application, the concentration of the microbial strain in the treated plant, plant part, or the plant's surroundings (e.g., immediate soil layer or rhizosphere) is at least 2×, 5×, 10×, 100×, 500×, or 1000× the concentration of the same microbial strain found in nature or detected in an untreated control plant, plant part, or the control plant's surroundings. In some embodiments of the above method, a microbial strain is applied to the plant, plant part, or to the plant's surroundings (e.g., immediate soil layer or rhizosphere) in a culture or a composition at a concentration of at least 1×10² CFU/mL. In some embodiments, concentration ranges are from about 1×10² to about 1×10¹⁰ CFU/mL, such as the concentrations ranging from 1×10⁵ to 1×10⁹ CFU/mL. In some embodiments, application of a microbial strain (PGPM) as described herein to a plant, plant part, or to the plant's surroundings (e.g., immediate soil layer or rhizosphere) in a culture or a composition at a concentration that is at least 1×10⁶ CFU/mL leads to a concentration of the microbial strain in the treated plant, plant part or the plant's surroundings that is at least 2× the amount of the strain found in an untreated plant or its surroundings.

Other embodiments provide a non-naturally occurring plant. In some embodiments, the non-naturally occurring plant is artificially infected with one or more microbial strains (PGPMs) according to the present embodiments. Further provided in some embodiments of this aspect is a plant seed, reproductive tissue, vegetative tissue, regenerative tissue, plant part or progeny of a plant.

### DESCRIPTION OF THE DRAWINGS

FIG. 1 shows yield results from field experiment described in Example 3.

### BRIEF DESCRIPTION OF THE SEQUENCES

### Detailed Description

Unless otherwise defined, all terms of art, notations and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed by those skilled in the art.

The singular form “a”, “an”, and “the” include plural references unless the context clearly dictates otherwise. For example, the term “a cell” includes one or more cells, including mixtures thereof.

As used herein, an isolated strain of a microbe is a strain that has been removed from its natural milieu. As such, the term “isolated” does not necessarily reflect the extent to which the microbe has been purified. But, in different embodiments, an “isolated” culture has been purified at least 2× or 5× or 10× or 50× or 100× from the raw material from which it is isolated. As a non-limiting example, if a culture is isolated from soil as raw material, the organism can be isolated to an extent that its concentration in a given quantity of purified or partially purified material (e.g., soil) is at least 2× or 5× or 10× or 50× or 100× of that in the original raw material.

A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants as well as undesirable chemical contaminants.

As used herein, a “biologically pure” strain is intended to mean the strain separated from materials with which it is normally associated in nature. A strain associated with other strains, or with compounds or materials that it is not normally found with in nature, is still defined as “biologically pure.” A monoculture of a particular strain is, of course, “biologically pure.” In different embodiments, a “biologically pure” culture has been purified at least 2× or 5× or 10× or 50× or 100× or 1000× or higher (to the extent considered feasible by a skilled person in the art) from the material with which it is normally associated in nature. As a non-limiting example, if a culture is normally associated with soil, the organism can be biologically pure to an extent that its concentration in a given quantity of purified or partially purified material with which it is normally associated (e.g. soil) is at least 2× or 5× or 10× or 50× or 100×, or 1000× or higher (to the extent considered feasible by a skilled person in the art) that in the original unpurified material.

As used herein, the term “enriched culture” of an isolated microbial strain refers to a microbial culture wherein the total microbial population of the culture contains more than 50%, 60%, 70%, 80%, 90%, or 95% of the isolated strain.

The term “culturing”, as used herein, refers to the propagation of organisms on or in media of various kinds. Suitable media are known to a person with ordinary skill in the art.

A “composition” as used herein means a combination of an active agent (e.g., a PGPM or microbial strain described herein) and at least one other compound, carrier, or composition, which can be inert (for example, a detectable agent or label or liquid carrier) or active, such as, but not limited to, a fertilizer, nutrient, or pesticide. A microbial composition refers to a composition comprising at least one microbial species.

Ribosomes, which are comprised of numerous ribosomal proteins and three ribosomal RNA (rRNA) molecules, are a key component of protein synthesis. The 16S subunit rRNA, which is encoded by the 16S rRNA gene, has been the focus of much attention in microbial phylogenetic studies. The 16S rRNA gene sequence is highly conserved between taxonomic groups, yet also possesses regions that are highly polymorphic. Moreover, the rate of change in the RNA sequence is thought to have been relatively constant over evolutionary time, enabling scientists to determine the relative relatedness of different organisms.

An “effective amount,” as used herein, is an amount sufficient to effect beneficial and/or desired results. An effective amount can be administered in one or more administrations. In terms of treatment, inhibition or protection, an effective amount is that amount sufficient to ameliorate, stabilize, reverse, slow or delay progression of a target infection, abiotic stress, or disease state. The expression “effective microorganism” used herein in reference to a microorganism is intended to mean that the subject strain exhibits a degree of promotion of plant health, growth and/or yield or a degree of inhibition of a pathogenic disease that exceeds, at a statistically significant level, that of an untreated control. In some instances, the expression “an effective amount” is used herein in reference to that quantity of microbial treatment which is necessary to obtain a beneficial or desired result relative to that occurring in an untreated control under suitable conditions of treatment as described herein. For example, the expression “an agriculturally effective amount” is used herein in reference to that quantity of microbial treatment which is necessary to obtain an agriculturally beneficial or desired result relative to that occurring in an untreated control under suitable conditions of treatment as described herein. The effective amount of an agricultural formulation or composition that should be applied for the improvement of plant health, growth and/or yield, for the control of, e.g., insects, plant diseases, or weeds, can be readily determined via a combination of general knowledge of the applicable field.

A “nutrient” as used herein means a compound or composition that is able to provide one or more nutrient elements to plants. In some embodiments, a nutrient provides one or more nutrient elements selected from nitrogen (N), phosphorus (P), potassium (K), calcium (Ca), magnesium (Mg), sulfur (S), iron (Fe), manganese (Mn), zinc (Zn), copper (Cu), nickel (Ni), boron (B) and molybdenum (Mo) to the plants. In some embodiments, a nutrient as used herein provides at least one of nitrogen (N), phosphorus (P) and potassium (K) to the plants. In some embodiments, a nutrient provides at least one of calcium (Ca), magnesium (Mg) and sulfur (S) to the plants. In some embodiments, a nutrient of the embodiments of this application provides at least one of iron (Fe), manganese (Mn), zinc (Zn), copper (Cu), nickel (Ni), boron (B) and molybdenum (Mo) to the plants. In some embodiments, a nutrient is a compound or composition that promotes the plant uptake of one or more nutrient elements selected from nitrogen (N), phosphorus (P), potassium (K), calcium (Ca), magnesium (Mg), sulfur (S), iron (Fe), manganese (Mn), zinc (Zn), copper (Cu), nickel (Ni), boron (B) and molybdenum (Mo), from the soil.

A “fertilizer” as used herein means a compound or composition that is added to plants or soil to improve plant health, growth and/or yield. In some embodiments, a fertilizer improves plant health, growth and/or yield by providing a nutrient (such as the ones described herein) to the plant. Fertilizers include, but are not limited to, inorganic fertilizers, organic (or natural) fertilizers, granular fertilizers and liquid fertilizers. Granular fertilizers are solid granules, while liquid fertilizers are made from water soluble powders or liquid concentrates that mix with water to form a liquid fertilizer solution. In some embodiments, plants can quickly take up most water-soluble fertilizers, while granular fertilizers may need a while to dissolve or decompose before plants can access their nutrients. High-tech granular fertilizers have “slow-release,” “timed-release,” or “controlled-release” properties, synonymous terms meaning that they release their nutrients slowly over a period of time. Organic fertilizer may come from a organic sources such as, but not limited to, compost, manure, blood meal, cottonseed meal, feather meal, crab meal, or others, as opposed to synthetic sources. There are also some natural fertilizers that are not organic, such as Greensand, which contain potassium, iron, calcium, and other nutrients. Organic fertilizers depend on the microbes in the soil to break them down into digestible bits for plantsInorganic fertilizers are also known as synthetic or artificial fertilizers. Inorganic fertilizers are manufactured.

A “bacteriostatic” compound or agent, or a bacteriostat (abbreviated Bstatic), is a biological or chemical agent that stops bacteria from growing and reproducing, while not necessarily harming them otherwise. An “acaricide” means a compound or composition that increases the mortality of, or materially inhibits the growth, reproduction, or spread of undesired acarids, including but not limited to dust mites. A “bactericide” means a compound or composition that increases the mortality of, or materially inhibits the growth, reproduction, or spread of undesired bacteria, such as (but not limited to) those unfavorable for the plant growth. A “fungicidal” refers to a compound or composition that increases the mortality of, or materially inhibits the growth, reproduction, or spread of undesired fungi, such as (but not limited to) those unfavorable for the plant growth. A “nematicide” refers to a compound or composition that increases the mortality of, or materially inhibits the growth, reproduction, or spread of undesired nematodes. A “insecticide” refers to a compound or composition that increases the mortality of, or materially inhibits the growth, reproduction, or spread of undesired insects, such as (but not limited to) those that are harmful for the plant growth. A “microbicide” refers to a compound or composition that increases the mortality of, or materially inhibits the growth, reproduction, or spread of undesired microbes, such as (but not limited to) those that are harmful for the plant growth. A “pesticide” refers to a compound or composition that increases the mortality of, increases plant resistance to, materially inhibits the growth of, materially inhibits the reproduction of, or materially inhibits the spread of undesired pests, such as (but not limited to) those that are harmful for the plant growth.

A “carrier” as used herein refers to a substance or a composition that support the survival of the microbes. Such carriers may be either organic or non-organic. In some embodiments, a carrier may be an agriculturally accepted carrier.

“Seed priming” or “priming of seed” means controlling the hydration level within seeds so that the metabolic activity necessary for germination can occur but elongation by the embryonic axis, i.e. usually radicle emergence, is prevented. Different physiological activities within the seed occur at different moisture levels (Leopold and Vertucci, 1989, Moisture as a regulator of physiological reactions in seeds. In: Seed Moisture, eds. P. C. Stanwood and M. B. McDonald. CSSA Special Publication Number 14. Madison, Wis.: Crop Science Society of America, pp. 51-69; Taylor, 1997, Seed storage, germination and quality. In: The Physiology of Vegetable Crops, ed. H. C. Wien. Wallingford, U.K.: CAB International, pp. 1-36). The last physiological activity in the germination process is radicle emergence. The initiation of radicle emergence requires a high seed water content. By limiting seed water content, all the metabolic steps necessary for germination can occur without the irreversible act of radicle emergence. Prior to radicle emergence, the seed is considered desiccation tolerant, thus the primed seed moisture content can be decreased by drying. After drying, primed seeds can be stored until time of sowing. For example, in some embodiments, a plant seed is exposed or placed in contact with a microbial strain or a culture thereof, or a composition according to the embodiments of this application during the hydration treatment of seed priming. In some embodiments, the exposure or contact of a plant seed with the microbial strain or a culture thereof or a composition of the embodiments of this application, during the priming process improves seed germination performance, later plant health, plant growth, and/or final plant yield.

As used herein, an “endophyte” is an endosymbiont that lives within a plant for at least part of its life. Endophytes may be transmitted either vertically (directly from parent to offspring) or horizontally (from individual to unrelated individual). In some embodiments, vertically-transmitted fungal endophytes are asexual and transmit from the maternal plant to offspring via fungal hyphae penetrating the host's seeds. Bacterial endophytes can also be transferred vertically from seeds to seedlings (Ferreira et al., FEMS Microbiol. Lett. 287:8-14, 2008). In some embodiments, horizontally-transmitted endophytes are typically sexual, and transmit via spores that can be spread by wind and/or insect vectors. Microbial endophytes of crop plants have received considerable attention with respect to their ability to control disease and insect infestation, as well as their potential to promoting plant growth. For instance, some microbial strains described herein may be able to establish as endophytes in plants that come in contact with them. Such microbial strains are microbial endophytes.

The term “pathogen” as used herein refers to an organism such as an alga, an arachnid, a bacterium, a fungus, an insect, a nematode, a parasitic plant, a protozoan, a yeast, or a virus capable of producing a disease in a plant or animal. The term “phytopathogen” as used herein refers to a pathogenic organism that infects a plant. A “pathogenic disease” is a disease, such as a plant disease, that is caused by at least one pathogen. A “phytopathogenic disease” is a disease, such as a plant disease, that is caused by at least one phytopathogen. Some pathogens that may cause plant pathogenic diseases include, but are not limited to, Colletotrichum, Fusarium, Gibberella, Monographella, Penicillium, and Stagnospora organisms.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

In some embodiments a polypeptide comprising a nucleotide sequence having at least about 80%, 81%, 82%, 83%, 84%, 85%, 86%, 87%, 88%, 89%, 90%, 91%, 92%, 93%, 94%, 95%, 96%, 97%, 98%, 99% or greater percent sequence identity across the entire length of the nucleotide sequence of any one of SEQ ID NOS: 1-474.

As used herein in reference to a nucleic acid and polypeptide, the term “variant” is used herein to denote a polypeptide, protein or polynucleotide molecule with some differences, generated synthetically or naturally, in their amino acid or nucleic acid sequences as compared to a reference polypeptide or polynucleotide, respectively. For example, these differences include substitutions, insertions, deletions or any desired combinations of such changes in a reference polypeptide or polypeptide. Polypeptide and protein variants can further consist of changes in charge and/or post-translational modifications (such as glycosylation, methylation. phosphorylation, etc.).

The term “variant”, when used herein in reference to a microorganism, is a microbial strain having identifying characteristics of the species to which it belongs, while having at least one nucleotide sequence variation or identifiably different trait with respect to the parental strain, where the trait is genetically based (heritable). In some embodiments, a variant may be induced from a microorganism through mutagenesis.

“PGPM” refers to plant-growth promoting microorganisms (or microbes). In some embodiments, PGPMs not only can promote plant health, growth and/or yield, but also can survive and multiply in microhabitats associated with the root surface, in competition with other microbiota, and/or are able to colonize the root, at least for the time needed to express their plant promotion and/or protection activities. In some embodiments, microbial strains whose 16S rRNA gene comprises a nucleic acid sequence selected from the SEQ ID NOs.: 1-474, are PGPMs and variants or progeny thereof.

The PGPMs, isolates, cultures, compositions or synthetic consortia promote or enhance plant health, growth or yield, and/or have plant growth-promoting activity. The term “plant growth-promoting activity”, as used herein, encompasses a wide range of improved plant properties, including, for example without limitation, improved nitrogen fixation, improved root development, increased leaf area, increased plant yield, increased seed germination, increased photosynthesis, or an increase in accumulated biomass of the plant. In some embodiments, the microbial strains, isolates, cultures, compositions or synthetic consortia as described herein improves stress tolerance (e.g., tolerance to drought, flood, salinity, heat, pest), improves nutrient uptake, plant heath and vigor, improves root development, increases leaf area, increases plant yield, increases seed germination, or an increase in accumulated biomass of the plant. In some embodiments, the microbial strains, isolates, cultures, compositions or synthetic consortia as described herein increase the size or mass of a plant or parts thereof, as compared to a control plant, or parts thereof or as compared to a predetermined standard. In some embodiments, the microbial strains, isolates, cultures, compositions or synthetic consortia as described herein promote plant growth by promoting seed germination, as compared to a control seed. In some embodiments, the microbial strains, isolates, cultures, compositions or synthetic consortia as described herein improve the health, vigor, and/or yield of a plant, as compared to a control plant.

As used herein, the term “yield” or “grain yield” refers to the amount of harvestable plant material or plant-derived product and is normally defined as the measurable produce of economic value of a crop. For crop plants, yield also means the amount of harvested material per acre or unit of production. Yield may be defined in terms of quantity or quality. The harvested material may vary from crop to crop, for example, it may be seeds, above ground biomass, roots, fruits, cotton fibers, any other part of the plant, or any plant-derived product which is of economic value. The term yield also encompasses yield potential, which is the maximum obtainable yield. Yield may be dependent on a number of yield components, which may be monitored by certain parameters. These parameters are well known to persons skilled in the art and vary from crop to crop. The term yield also encompasses harvest index, which is the ratio between the harvested biomass over the total amount of biomass.

As described herein, Event DP-202216-6 refers to the same maize event DP-202216-6. The protein encoded by the Maize MADS box ZmM28 gene in the plasmid PHP40099 or the Event DP-202216-6 is also referred to as AG099 protein (SEQ ID NO: 476) and the corresponding DNA sequence as AG099 gene or AG099 DNA (SEQ ID NO: 476).

Traits that may confer increased grain yield include, but are not limited to, a Maize MADS box ZmM28 gene, a 1-AminoCyclopropane-1-Carboxylate Deaminase-like Polypeptide (ACCDP (U.S. Pat. No. 8,097,769), a maize zinc finger protein gene (Zm-ZFP1) (US Patent Application Publication Number 2012/0079623), a maize lateral organ boundaries (LOB) domain protein (Zm-LOBDP1) (US Patent Application Publication Number 2012/0079622), a VIM1 (Variant in Methylation 1)-like polypeptide or a VTC2-like (GDP-L-galactose phosphorylase) polypeptide or a DUF1685 polypeptide or an ARF6-like (Auxin Responsive Factor) polypeptide (WO 2012/038893), a Ste20-like polypeptide or a homologue thereof (EP 2431472), and a nucleoside diphosphatase kinase (NDK) polypeptides and homologs thereof (US Patent Application Publication Number 2009/0064373).

Compositions of this disclosure include a representative sample of seeds which was deposited as Patent Deposit No. PTA-124653 and plants, plant cells, and seed derived therefrom. Applicant(s) have made a deposit of at least 2500 seeds of maize event DP-202216-6 (Patent Deposit No. PTA-124653) with the American Type Culture Collection (ATCC), Manassas, Va. 20110-2209 USA, on Jan. 12, 2018. These deposits will be maintained under the terms of the Budapest Treaty on the International Recognition of the Deposit of Microorganisms for the Purposes of Patent Procedure. The seeds deposited with the ATCC on Jan. 12, 2018 were taken from a representative sample deposit maintained by Pioneer Hi-Bred International, Inc., 7250 NW 62^{nd} Avenue, Johnston, Iowa 50131-1000. Access to this deposit will be available during the pendency of the application to the Commissioner of Patents and Trademarks and persons determined by the Commissioner to be entitled thereto upon request. Upon issuance of a patent, this deposit of seed of maize Event DP-202216-6 is intended to meet all the necessary requirements of 37 C.F.R. §§ 1.801-1.809, and will be maintained in the ATCC depository, for a period of 30 years, or 5 years after the most recent request, or for the enforceable life of the patent, whichever is longer, and will be replaced if it becomes nonviable during that period. Applicant(s) have no authority to waive any restrictions imposed by law on the transfer of biological material or its transportation in commerce. Unauthorized seed multiplication prohibited. The seed may be regulated under one or more applicable National, State or other local regulations and ordinances imposed by one or more competent governmental agencies.

In some embodiments, the microbial strains, isolates, cultures and compositions comprising a plant or plant seed, wherein the plant or plant seed comprises at least one modified or transgenic grain yield enhancing trait have a grain yield improvement that is an at least 2% increase, at least 3% increase, at least 4% increase, at least 5% increase, at least 10% increase, at least 15% increase, at least 20%, at least 25% increase, at least 50% increase, at least 75% increase, or at least a 100% increase in the property being measured compared to a control plant. Thus, as non-limiting examples, the microbial strains, isolates, cultures and compositions according to the the embodiments of this application may produce an above stated percentage increase in nitrogen fixation, or an above stated increase in total root weight, or in leaf area or in plant product yield (e.g., an above stated percentage increase in plant product weight), or an increased percentage of seeds that germinate within 10 days or 14 days or 30 days, or rate of photosynthesis (e.g., determined by CO₂ consumption) or accumulated biomass of the plant (e.g., determined by weight and/or height of the plant). The plant product is the item—usually but not necessarily—a food item produced by the plant.

A “control plant”, as used herein, provides a reference point for measuring changes in phenotype of the subject plant, and may be any suitable plant cell, seed, plant component, plant tissue, plant organ or whole plant. A control plant may comprise, for example (but not limited to), (a) a wild-type plant or cell, i.e., of the same genotype as the starting material for the genetic alteration which resulted in the subject plant or cell; (b) a plant or cell of the genotype as the starting material but which has been transformed with a null construct (i.e., a construct which has no known effect on the trait of interest, such as a construct comprising a reporter gene); (c) a plant or cell which is a non-transformed segregant among progeny of a subject plant or cell; (d) a plant or cell which is genetically identical to the subject plant or cell but which is not exposed to the same treatment (e.g., inoculant treatment) as the subject plant or cell; (e) the subject plant or cell itself, under conditions in which the gene of interest is not expressed; or (f) the subject plant or cell itself, under conditions in which it has not been exposed to a particular treatment such as, for example, an inoculant or combination of inoculants, microbial strains, and/or other chemicals.

“Inoculant” as used herein refers to any culture or preparation that comprises at least one microorganism. In some embodiments, an inoculant (sometimes as microbial inoculant, or soil inoculant) is an agricultural amendment that uses beneficial microbes, such as PGPMs, (including, but not limited to endophytes) to promote plant health, growth and/or yield. Many of the microbes suitable for use in an inoculant may form symbiotic relationships with the target crops where both parties benefit (mutualism).

Competitive fitness refers to the fitness of the microbes to compete with their neighbors for space and resources. Fitness means the ability or propensity of a given genotype (e.g., a 16S rRNA gene sequence) to both survive and reproduce in a given environment.

Biofertilizers designate the biological products which contain microorganisms providing direct and/or indirect gains in plant health, growth and/or yield.

A bioreactor refers to any device or system that supports a biologically active environment. As described herein a bioreactor is a vessel in which microorganisms including the microorganism of the embodiments of this application can be grown.

Diverse plant-associated microorganisms, including, but not limited to, many rhizobacterial species, can positively impact plant health and physiology in a variety of ways. These beneficial microbes are generally referred to as PGPMs, such as plant growth-promoting bacteria (PGPB) or plant growth-promoting rhizosphere (PGPR). Isolated strains of microorganisms have been reported to have plant growth-promoting activity and/or biocontrol activity, and new genera and species with similar activities are still being discovered. Additionally, within some bacterial genera, multiple species and subspecies of biocontrol agents have been identified and can be found across multiple spatial scales, from the global level to farm level, and even on single plants. Furthermore, it has been reported that some individual microbial isolates may display biocontrol and/or plant growth-promoting activity not only on the plants or crops from which they were obtained but also on other crops. This indicates the generalist nature of some genotypes, especially those with a wide geographic distribution. If introduced in sufficient numbers and active for a sufficient duration, a single microbial population can have a significant impact on plant health.

The embodiments disclosed include new microbial strains that are PGPMs. In some embodiments, a microbial strain disclosed herein comprises a nucleotide sequence selected from SEQ ID NOs.: 1-474 or a variant or progenty thereof. In some embodiments, the microbial strain comprises a 16S rRNA gene comprising a nucleotide sequence selected from SEQ ID NOs.: 165-474. In some embodiments, the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% sequence identity to any one of the nucleotide sequences as set forth in SEQ ID NOs.: 1-474. Some embodiments provide a genus of plant growth-promoting microorganisms comprising any of the DNA sequences described herein and which enhances the health, growth and/or yield of a plant, as described herein.

In some embodiments, the microbial strain is selected from P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338) or a strain derived from any one of these strains. The deposits were made under the provisions of the Budapest Treaty on the International Recognition of the Deposit of Microorganisms for the Purposes of Patent Procedure. Further, these deposits will be maintained under the terms of the Budapest Treaty on the International Recognition of the Deposit of Microorganisms for the Purposes of Patent Procedure. Access to these deposits will be available during the pendency of the application to the Commissioner of Patents and Trademarks and persons determined by the Commissioner to be entitled thereto upon request. Upon allowance of any claims in the application, the Applicant will make available to the public, pursuant to 37 C.F.R. § 1.808, sample(s) of the deposits. The deposits will be maintained in the NRRL depository, which is a public depository, for a period of 30 years, or 5 years after the most recent request, or for the enforceable life of the patent, whichever is longer, and will be replaced if it becomes nonviable during that period.

Some embodiments also provide isolates and cultures of the microbial strains as described herein, and compositions and synthetic consortia comprising various combinations of those microbial strains, isolates or cultures and a plant or plant seed, wherein the plant or plant seed comprises at least one modified or transgenic grain yield enhancing trait.

In some embodiments, the PGPMs, when applied to seed, plant surfaces, plant parts, or soil, colonizes rhizosphere and/or the interior of the plant and promotes growth of the host plant. In some embodiments, PGPMs are biofertilizers. In some embodiments, the PGPMs are microbial fertilizers, which supply the plant with nutrients and thereby can promote plant growth in the absence of pathogen pressure. In some embodiments, the PGPMs may directly promote plant growth and/yield through mechanisms, including, but not limited to, ability to produce or change the concentration of plant hormones; asymbiotic nitrogen fixation; and/or solubilization of mineral phosphate and other nutrients.

In some embodiments, PGPMs may affect the plant growth and development as phytostimulators. For example, some PGPMs described herein have the ability to produce or change the concentration of plant hormones, including, but not limited to the five classical phytohormones, i.e., auxin, ethylene, abscisic acid, cytokinin, and gibberellin. Some PGPMs may also produce enzymes or secondary metabolites that affect phytohormone production in plants. In some embodiments, PGPMs may have the ability to produce or change the concentration of other hormones as well as certain volatile organic compounds (VOCs) and the cofactor pyrrolquinoline quinone (PQQ), thereby stimulating plant growth and/or yield.

In some embodiments, PGPMs may affect the plant growth and development by modifying nutrient availability or uptake. The PGPMs may alter nutrient uptake rates, for example, by direct effects on roots, by effects on the environment which in turn modify root behavior, and by competing directly for nutrients. Some factors by which PGPMs described herein may play a role in modifying the nutrient use efficiency in soils include, for example, root geometry, nutrient solubility, nutrient availability by producing plant congenial ion form, partitioning of the nutrients in plant and utilization efficiency. For example, a low level of soluble phosphate can limit the growth of plants. Some plant growth-promoting microbes are capable of solubilizing phosphate from either organic or inorganic bound phosphates, thereby facilitating plant growth.

In some embodiments, PGPMs may affect the plant growth and development as plant stress controllers. For example, some PGPMs may control and/or reduce several types of plant stress, including, but not limited to, stress from the effects of phytopathogenic bacteria, stress from polyaromatic hydrocarbons, stress from heavy metal such as Ca′ and Ni′, and stress from salt and severe weather conditions (e.g., drought or flood).

In some embodiments, PGPMs may promote plant health, growth and/or yield directly by controlling phytophathogens or pests in plants. In some embodiments, PGPMs described herein exhibit one or more mechanisms of biological disease control, most of which involve competition and production of metabolites that affect the pathogen directly. Examples of such metabolites include antibiotics, cell wall-degrading enzymes, siderophores, and HCN. It is noteworthy to state that different mechanisms may be found in a single PGPM strain and act simultaneously. In some embodiments, PGPMs may affect the plant growth and development by producing extracellular siderophores. Some PGPMs described herein may secrete low molecular weight, high affinity ferric-chelating microbial cofactors that specifically enhance their acquisition of iron by binding to membrane bound siderophore receptors. Siderophores are small, high-affinity chelators that bind Fe, making it more (or less) available to certain member of natural microflora. For example, a siderophore may make Fe more available to a plant or microbe that possesses the ability to recognize and import the specific siderophore molecular structure. Many different siderophore types and structures exist with different Fe-binding affinities. Furthermore, exchange of Fe from a siderophore with low Fe-binding affinity to one with higher Fe-binding affinity is known to occur which may further influence Fe availability to any given organism. One of the siderophores produced by some pseudomonad PGPMs is known as pseudobactin that inhibits the growth of Erwinia cartovora (causal organism for soft-rot of potato) (see, e.g., Kloepper et al. Current Microbiol. 4: 317-320, 1980). Additions of pseudobactin to the growth medium inhibited soft-rot infection and also reduced the number of pathogenic fungi in the potato plant along with a significant increase in potato yield. Most evidence to support the siderophore theory of biological control by PGPM comes from work with the pyoverdines, one class of sideophores that comprises the fluorescent pigments of fluorescent pseudomonads (Demange et al. in Iron Transport in Microbes, Plants and Animals, pp 167-187, 1987). According to the siderophore theory, pyoverdines demonstrate certain functional strain specificity which is due to selective recognition of outer membrane siderophore receptors (Bakker et al. Soil Biology and Biochemistry 19: 443-450, 1989). Production of siderophore(s) may modulate the fitness and/or growth of other strains. In addition to inhibiting certain strains (e.g., Erwinia), production of siderophore(s) can also support the fitness/growth of other microbial strains that possess receptors for a given siderophore but are unable to synthesize the molecule themselves.

In some embodiments, the PGPMs may act indirectly on the plant by increasing the competitive fitness of a second microbial strain (e.g., another PGPM) by, e.g., providing nutrients, metabolites and/or siderophores (and/or by any other benefiting mechanism as described herein) to the second microbial strain. In some embodiments, the PGPMs may act indirectly on the plant by increasing the competitive fitness of a second microbial strain (e.g., another PGPM) by, e.g., providing nutrients, metabolites and/or siderophores (and/or by any other benefiting mechanism as described herein) to the second microbial strain, and/or by decreasing the competitive fitness of a third microbial strain that inhibits, competes with, or excludes or otherwise has a negative impact on the fitness of the second microbial strain.

In some embodiments, the PGPMs are biocontrol agents of plant diseases by activating chemical and/or physical defenses of the host plants, i.e., inducing induced systemic resistance (ISR) or systemic acquired resistance (SAR). In some embodiments, induction of resistance promoted by PGPMs of the present embodiments is active and signaling in the route of salicylic acid with induction of proteins related to the pathogenesis (PR-proteins) or route of the jasmonic acid and ethylene. Sometimes, when the PGPMs colonize the root system, constituents of the microorganism cell molecules act as a biochemical signal, and the genes that encode for the synthesis of the PR-proteins are activated. In addition to PR-proteins, plants produce other enzymes of the defense, including peroxidases, phenylalanine ammonia-lyse (PAL), and polyphenoloxidase (PPO). Peroxidase and PPO are catalysts in the formation of lignin. PAL and other enzymes are involved in the formation of phytoalexins. In some embodiments, the PGPMs described herein induce plant resistance to diseases by increasing peroxidases, PPO and/or PAL production.

In some embodiments, the PGPMs of the embodiments of this application promote the plant health, growth and/or yield via one or more of the mechanisms as described herein.

In some embodiments, the PGPMs of the embodiments of this application are biofertilizers or biocontrol agents, which are compatible with organic farming.

Other aspects of the present embodiments contemplate isolated and/or cultured PGPMs. In one aspect, an embodiment provides isolated microbial strains (or PGPMs), isolated cultures thereof, biologically pure cultures thereof, and enriched cultures thereof. In some embodiments, the microbial isolate or culture comprises at least one microbial strain, wherein the 16S rRNA gene of the microbial strain comprises a nucleotide sequence selected from SEQ ID NOs.: 1-474. In some embodiments, the microbial isolate or culture comprises at least one microbial strain, wherein the 16S rRNA gene of the microbial strain comprises a nucleotide sequence selected from SEQ ID NOs.: 165-474. In some embodiments, the microbial isolate or culture comprises at least one microbial strain, wherein the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, or at least 99.5% sequence identity to any one of the nucleotide sequences as set forth in SEQ ID NOs.: 1-474.

Some embodiments provide a microbial isolate or culture thereof comprising at least microbial strain selected from: P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338) or a strain derived from any one of these strains. The microbial isolates or cultures promote the plant health, growth and/or yield, e.g., via one or more of the mechanisms as described herein in a plant or plant seed, wherein the plant or plant seed comprises at least one modified or transgenic grain yield enhancing trait.

### Microbiological Compositions

Embodiments of this application provide a microbial composition that comprises a PGPM or microbial strain, such as a microbial strain selected from those described herein, or a culture thereof and a plant or plant seed, wherein the plant or plant seed comprises at least one modified or transgenic grain yield enhancing trait. In some embodiments, the microbial composition comprises a microbial strain, wherein the 16S rRNA gene of said strain comprises a sequence selected from the group consisting of SEQ ID NOs.: 1-474, or a culture thereof.

In some embodiments, a microbial composition comprises at least one microbial strain, wherein the 16S rRNA gene of said strain comprises a sequence selected from the group consisting of SEQ ID NOs.: 1-474, or a culture thereof. In some embodiments, the microbial composition comprises at least one microbial strain, wherein the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, or at least 99.5% sequence identity to any one of the nucleotide sequences as set forth in SEQ ID NOs.: 1-474, or a culture thereof.

In some embodiments, a microbial composition comprising a plant or plant seed and at least two, at least three, at least four, at least five, at least ten, or at least 20 microbial strains, wherein the 16S rRNA gene of said strain comprises a sequence selected from the group consisting of SEQ ID NOs.: 1-474, or a culture thereof, and wherein the plant or plant seed comprises at least one modified or transgenic grain yield enhancing trait. In some embodiments, the microbial composition comprises at least two, at least three, at least four, at least five, at least ten, or at least 20 microbial strains, wherein the 16S rRNA gene of the microbial strain comprises a nucleotide sequence that exhibits at least 85%, at least 90%, at least 91%, at least 92%, at least 93%, at least 94%, at least 95%, at least 96%, at least 97%, at least 98%, at least 99%, or at least 99.5% sequence identity to any one of the nucleotide sequences as set forth in SEQ ID NOs.: 1-474, or a culture thereof.

In some embodiments, the microbial composition comprises one or more microbial strains selected from P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), and any combination thereof, and strains derived therefrom, or cultures thereof and a plant or plant seed, wherein the plant or plant seed comprises at least one modified or transgenic grain yield enhancing trait. In some embodiments, the microbial composition comprises at least two, at least three, at least four, at least five, at least ten, or at least 20 microbial strains disclosed herein and a plant or plant seed, wherein the plant or plant seed comprises at least one modified or transgenic grain yield enhancing trait. In another embodiment, the microbial composition comprises a plurality of strains disclosed herein.

In some embodiments, the microbial composition comprises at least one, at least two, at least three, at least four, at least five, at least ten, or at least 20 microbial strains selected from P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or strains derived therefrom, or cultures thereof and a plant or plant seed, wherein the plant or plant seed comprises at least one modified or transgenic grain yield enhancing trait. In another embodiment provides a composition comprising one or more Arthrobacter microbial strains. In another embodiment provides a composition comprising one or more Arthrobacter globiformis microbial strains.

In another embodiment provides a composition comprising a synthetic microbial consortium. In some embodiments, a synthetic consortium comprises: (a) a first set of microbes comprising one or more microbes that promote plant health, growth, and/or yield; and (b) a second set of microbes comprising one or more microbes that increase (directly or indirectly) the competitive fitness of one or more of the microbes of the first set of microbes in step (a); wherein the first and the second sets of microbes are combined into a single mixture as a synthetic consortium. In one embodiment, the synthetic consortium further comprises microbial strains not found together in nature. In another embodiment, the synthetic consortium comprises microbial strains not found in comparable concentrations relative to one another in nature. In some embodiments of a synthetic consortium, one or more microbes of the first set of microbes ((a) above) enhance nutrient availability and/or nutrient uptake of a plant. In some embodiments of a synthetic consortium, one or more microbes in the first set of microbes ((a) above) modulate plant hormone levels. In some embodiments of a synthetic consortium, one or more microbes in the first set of microbes ((a) above) demonstrate one or more of the activities selected from nitrogen fixation, IAA production, ACC deaminase activity, phosphate solubilization, and/or iron solubilization (and/or any other activities from which plant health, growth, and/or yield may be benefited). In some embodiments of a synthetic consortium, one or more microbes of the first set of microbes ((a) above) inhibit or suppress a plant pathogen (e.g., as a biological pesticide such as one selected from those described herein). In some embodiments of a synthetic consortium, one or more microbes in the second set of microbes ((b) above) directly increase the competitive fitness of one or more microbes in the first set of microbes ((a) above). In some embodiments, one or more microbes in the second set of microbes produce a metabolite that enhances the competitive fitness of one or more microbes in the first set of microbes. For example, one or more microbes in the second set of microbes produce a siderophore that enhances iron acquisition of one or more of the microbes in the first set of microbes. In some embodiments of a synthetic consortium, one or more microbes in the second set of microbes ((b) above) decrease the competitive fitness of a microorganism that is distinct from the microbes of the first or the second sets of microbes ((a) or (b) above), and potentially detrimental to (e.g., by inhibiting, competing with, excluding, or otherwise having a negative impact on) the fitness of one or more microbes in the first set of microbes ((a) above). In some embodiments of a synthetic consortium, one or more microbes in the second set of microbes ((b) above) produce a metabolite that is bactericidal, bacteriostatic or otherwise modulates growth of a microorganism that is distinct from the microbes of the first and the second sets of microbes, and that is detrimental to (e.g., by inhibiting, competing with, excluding, or otherwise having a negative impact on) the fitness of one or more microbes in the first set of microbes ((a) above). For example, one or more of the microbes in the second set of microbes ((b) above) produce a siderophore that inhibits the growth or fitness of a microorganism that is potentially detrimental to one or more microbes in the first set ((a) above). Thus, the function of the second set of microbes is to directly or indirectly increase the fitness or competitive fitness of the first set of microbes. In some embodiments of a synthetic consortium, the first and second set of microbes are combined and supplemented with an inert formulary component. In some embodiments, the synthetic consortium and compositions thereof promotes or enhances the health, growth and/or yield of a plant. In some embodiments, the synthetic consortium or a composition thereof according to the present application is applied to a plant or plant seed, wherein the plant or plant seed comprises at least one modified or transgenic grain yield enhancing trait.

In some embodiments, the microbial compositions described herein, such as any of the microbial compositions described above, further comprise an agriculturally effective amount of an additional substance, compound or composition, such as, but not limited to, a nutrient, a fertilizer, an acaricide, a bactericide, a fungicide, an insecticide, a microbicide, a nematicide, a pesticide, or a combination thereof.

In some embodiments, the compositions are chemically inert; hence they are compatible with substantially any other constituents of the application schedule. The compositions may also be used in combination with plant growth affecting substances, such as fertilizers, plant growth regulators, and the like, provided that such compounds or substances are biologically compatible. The compositions may also be used in combination with biologically compatible pesticidal active agents as, for example, herbicides, nematocides, fungicides, insecticides, and the like.

In some embodiments, the microbial strains and compositions may furthermore be in the form of a mixture with synergists. Synergists are compounds by which the activity of the active compositions is increased without it being necessary for the synergist added to be active itself.

In some embodiments, the microbial strains and compositions may furthermore be in the form of a mixture with inhibitors (e.g., preservatives) which reduce the degradation of the active compositions after application in the habitat of the plant, on the surface of parts of plants or in plant tissues.

The active microbial strains and compositions may be used as a mixture with known fertilizers, acaricides, bactericides, fungicides, insecticides, microbicides, nematicides, pesticides, or combinations of any thereof, for example in order to widen the spectrum of action or to prevent the development of resistances to pesticides in this way. In many cases, synergistic effects, i.e., the activity of the mixture can exceed the activity of the individual components. A mixture with other known active compounds, such as growth regulators, safeners and/or semiochemicals is also contemplated.

In some embodiments, the compositions may include at least one chemical or biological fertilizer. The amount of at least one chemical or biological fertilizer employed in the compositions may vary depending on the final formulation as well as the size of the plant and seed to be treated. In some embodiments, the at least one chemical or biological fertilizer employed is about 0.1% w/w to about 80% w/w based on the entire formulation. In some embodiments, the at least one chemical or biological fertilizer is present in an amount of about 1% w/w to about 60%> w/w and in some embodiments about 10%> w/w to about 50% w/w.

The microbiological compositions optionally further include at least one biological fertilizer. Exemplary biological fertilizers that are suitable for use herein and can be included in a microbiological composition according to the embodiments of this application for promoting plant growth and/yield include microbes, animals, bacteria, fungi, genetic material, plant, and natural products of living organisms. In these compositions, the microorganism is isolated prior to formulation with an additional organism. For example, microbes such as but not limited to species of Achromobacter, Ampelomyces, Aureobasidium, Azospirillum, Azotobacter, Bacillus, Beauveria, Bradyrhizobium, Candida, Chaetomium, Cordyceps, Cryptococcus, Dabaryomyces, Delftia, Erwinia, Exophilia, Gliocladium, Herbaspirillum, Lactobacillus, Mariannaea, Microccocus, Paecilomyces, Paenibacillus, Pantoea, Pichia, Rhizobium, Saccharomyces, Sporobolomyces, Stenotrophomonas, Talaromyces, and Trichoderma can be provided in a composition with the microorganisms. Use of the microbiological compositions according to the present embodiments in combination with the microbial microorganisms disclosed in U.S. Patent Appl. Publication Nos. US20030172588_A1, US20030211119_A1, US20130276493, US20140082770; U.S. Pat. Nos. 7,084,331; 7,097,830; 7,842,494; PCT Appl. Nos. WO2010109436_A1, WO2013158900, and WO2013090628 is also contemplated.

In some embodiments, the methods and compositions disclosed herein may include at least one chemical or biological pesticide, acaricide, bactericide, fungicide, insecticide, microbicide, nematicide, or a combination thereof. The amount of at least one chemical or biological pesticide, acaricide, bactericide, fungicide, insecticide, microbicide, nematicide, or a combination thereof employed in the compositions can vary depending on the final formulation as well as the size of the plant and seed to be treated. In some embodiments, the at least one chemical or biological pesticide, acaricide, bactericide, fungicide, insecticide, microbicide, nematicide, or a combination thereof employed is about 0.1% w/w to about 80% w/w based on the entire formulation. In some embodiments, the at least one chemical or biological pesticide, acaricide, bactericide, fungicide, insecticide, microbicide, nematicide, or a combination thereof is present in an amount of about 1% w/w to about 60%> w/w and most preferably about 10%> w/w to about 50% w/w.

A variety of chemical pesticides and may be used. Exemplary chemical pesticides include those in the carbamate, organophosphate, organochlorine, and pyrethroid classes. Also included are chemical control agents such as, but not limited to, benomyl, borax, captafol, captan, chorothalonil, formulations containing copper; formulations containing dichlone, dicloran, iodine, zinc; fungicides such as but not limited to blastididin, cymoxanil, fenarimol, flusilazole, folpet, imazalil, ipordione, maneb, manocozeb, metalaxyl, oxycarboxin, myclobutanil, oxytetracycline, PCNB, pentachlorophenol, prochloraz, propiconazole, quinomethionate, sodium aresenite, sodium DNOC, sodium hypochlorite, sodium phenylphenate, streptomycin, sulfur, tebuconazole, terbutrazole, thiabendazole, thiophanate-methyl, triadimefon, tricyclazole, triforine, validimycin, vinclozolin, zineb, and ziram.

In some embodiments, the methods and compositions disclosed herein include at least one biological pesticide. Exemplary biological pesticides that are suitable for use herein and can be included in a microbiological composition for preventing a plant pathogenic disease include microbes, animals, bacteria, fungi, genetic material, plant, and natural products of living organisms. In these compositions, the microorganism is isolated prior to formulation with an additional organism. For example, microbes such as but not limited to species of Anthrobacter, Ampelomyces, Aureobasidium, Bacillus, Beauveria, Candida, Chaetomium, Cordyceps, Cryptococcus, Dabaryomyces, Erwinia, Exophilia, Gliocladium, Mariannaea, Paecilomyces, Paenibacillus, Pantoea, Pichia, Pseudomonas, Sporobolomyces, Streptomyces, Talaromyces, and Trichoderma can be provided in a composition with the microorganisms disclosed herein, with fungal strains of the Muscodor genus being preferred. Use of the microbiological compositions in combination with the microbial antagonists disclosed in U.S. Pat. Nos. 7,518,040; 7,601,346; and 6,312,940 is also contemplated.

Examples of fungi that may be combined with microbial strains and compositions in a composition include, without limitation, Muscodor species, Aschersonia aleyrodis, Beauveria bassiana (“white muscarine”), Beauveria brongniartii, Chladosporium herbarum, Cordyceps clavulata, Cordyceps en tomorrhiza, Cordyceps facis, Cordyceps gracilis, Cordyceps melolanthae, Cordyceps militaris, Cordyceps myrmecophila, Cordyceps ravenelii, Cordyceps sinensis, Cordyceps sphecocephala, Cordyceps subsessilis, Cordyceps unilateralis, Cordyceps variabilis, Cordyceps washingtonensis, Culicinomyces clavosporus, Entomophaga grylli, Entomophaga maimaiga, Entomophaga muscae, Entomophaga praxibulli, Entomophthora plutellae, Fusarium lateritium, Glomus species, Hirsutella citriformis, Hirsutella thompsoni, Metarhizium anisopliae (“green muscarine”), Metarhizium flaviride, Muscodor albus, Neozygitesfloridana, Nomuraea rileyi, Paecilomyces farinosus, Paecilomyces fumosoroseus, Pandora neoaphidis, Tolypocladium cylindrosporum, Verticillium lecanii, Zoophthora radicans, and mycorrhizal species such as Laccaria bicolor. Other mycopesticidal species will be apparent to those skilled in the art.

In still another embodiment, the PGPM compositions, consortia and methods disclosed herein can be used to treat a genetically modified plant or seed or a transgenic plant or seed. As used herein, the term “genetically modified” is intended to mean any species containing a genetic trait, loci, or sequence that was not found in the species or strain prior to manipulation. A genetically modified plant may be transgenic, cis-genic, genome edited, or bred to contain a new genetic trait, loci, or sequence. A genetically modified plant may be prepared by means known to those skilled in the art, such as transformation by bombardment, by a Cas/CRISPR or TALENS system, or by breeding techniques. As used herein, a “trait” is a new or modified locus or sequence of a genetically modified plant, including, but not limited to, a transgenic plant. A trait may provide herbicide or insect resistance to the genetically modified plant. As used herein, a “transgenic” plant, plant part, or seed refers to a plant, plant part, or seed containing at least one heterologous gene that allows the expression of a polynucleotide or polypeptide not naturally found in the plant. The heterologous gene in transgenic seed can originate, for example, from microorganisms of the species Bacillus, Rhizobium, Pseudomonas, Serratia, Trichoderma, Clavibacter, Glomus or Gliocladium.

A further embodiment relates to a method of increasing the durability of plant pest compositions comprising providing a plant protection composition to a plant or planted area, and providing the PGPM compositions, consortia and methods described herein to the plant or planted area, wherein the PGPM compositions, consortia and methods described herein have a different mode of action than the plant protection composition.

The present disclosure further provides methods and compositions that contain at least one of the isolated microbial strains or cultures thereof, such as any one of those described herein, and a carrier. The carrier may be any one or more of a number of carriers that confer a variety of properties, such as increased stability, wettability, dispersibility, etc. Wetting agents such as natural or synthetic surfactants, which can be nonionic or ionic surfactants or a combination thereof, can be included in a composition of the embodiments. Emulsions, such as water-in-oil emulsions can also be used to formulate a composition that includes at least one isolated microorganism of the present embodiments (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products (e.g., ground grain or beans, broth or flour derived from grain or beans), starch, sugar, or oil. The carrier may be an agricultural carrier. In certain preferred embodiments, the carrier is a seed, and the composition may be applied or coated onto the seed or allowed to saturate the seed.

In some embodiments, the agricultural carrier may be soil or plant growth medium. Other agricultural carriers that may be used include fertilizers, plant-based oils, humectants, or combinations thereof. In some embodiments, an agricultural carrier does not include only water as a carrier. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant parts, sugar cane bagasse, hulls or stalks from grain processing, ground plant material (“yard waste”), compost, or wood from building site refuse, sawdust or small fibers from recycling of paper, fabric, or wood. Other suitable agricultural carriers are known to those skilled in the art.

In some embodiments, the carrier suitable for the compositions described herein is an organic carrier. The organic carriers include, but are not limited to, peat, turf, talc, lignite, kaolinite, pyrophyllite, zeolite, montmorillonite, alginate, press mud, sawdust, and vermiculite. Talc is a natural mineral referred as steatite or soapstone composed of various minerals in combination with chloride and carbonate. Chemically it is referred as magnesium silicate and available as powder form from industries suited for wide range of applications. Talc has relative hydrophobicity, low moisture equilibrium, chemical inertness, reduced moisture absorption and it prevents the formation of hydrate bridges which enable longer storage periods. Peat (turf) is a carbonized vegetable tissue formed in wet conditions by decomposition of various plants and mosses. Peat is formed by the slow decay of successive layers of aquatic and semi aquatic plants, such as sedges, reeds, rushes, and mosses. Press mud is a byproduct of sugar industries. Vermiculite is a light mica-like mineral used to improve aeration and moisture retention. In some embodiments, compositions with organic carriers as described herein are suitable for organic farming. Other suitable organic carriers are known to those skilled in the art.

The microbiological compositions that comprise isolated microbial strains or cultures thereof may be in a variety of forms, including, but not limited to, still cultures, whole cultures, stored stocks of cells, mycelium and/or hyphae (particularly glycerol stocks), agar strips, stored agar plugs in glycerol/water, freeze dried stocks, and dried stocks such as lyophilisate or mycelia dried onto filter paper or grain seeds. As defined herein, “isolated culture” or grammatical equivalents as used in this disclosure and in the art is understood to mean that the referred to culture is a culture fluid, pellet, scraping, dried sample, lyophilisate, or section (for example, hyphae or mycelia); or a support, container, or medium such as a plate, paper, filter, matrix, straw, pipette or pipette tip, fiber, needle, gel, swab, tube, vial, particle, etc. that contains a single type of organism. An isolated culture of a microbial antagonist is a culture fluid or a scraping, pellet, dried preparation, lyophilisate, or section of the microorganism, or a support, container, or medium that contains the microorganism, in the absence of other organisms.

In some embodiments, the compositions are in a liquid form. For example, in the liquid form, e.g., solutions or suspensions, the microorganisms of the present embodiments may be mixed or suspended in water or in aqueous solutions. Suitable liquid diluents or carriers include water, aqueous solutions, petroleum distillates, or other liquid carriers.

In some embodiments, the compositions are in a solid form. For example, solid compositions can be prepared by dispersing the microorganisms of the embodiments in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

In one embodiment, the microbial composition promotes plant health, growth and/or yield via one or more mechanisms by which PGPMs function, as described herein. In some embodiments, the compositions contemplated herein enhance the growth and yield of crop plants by acting as microbial fertilizers, biocontrol agents of plant diseases, and/or inducers of plant resistance. The compositions, similarly to other biofertilizer agents, may have a high margin of safety because they typically do not burn or injure the plant. In some embodiments, a biocontrol agent comprises a bacterium, a fungus, a yeast, a protozoan, a virus, an entomopathogenic nematode, a botanical extract, a protein, a nucleic acid, a secondary metabolite, and/or an innoculant.

As described herein, enhancing plant growth and plant yield may be effected by application of one or more of the compositions to a host plant or parts of the host plant. The compositions can be applied in an amount effective to enhance plant growth or yield relative to that in an untreated control. The active constituents are used in a concentration sufficient to enhance the growth of the target plant when applied to the plant. Effective concentrations may vary depending upon various factors such as, for example, (a) the type of the plant or agricultural commodity; (b) the physiological condition of the plant or agricultural commodity; (c) the concentration of pathogens affecting the plant or agricultural commodity; (d) the type of disease injury on the plant or agricultural commodity; (e) weather conditions (e.g., temperature, humidity); and (f) the stage of plant disease. Typical application concentrations are of about 10 to 1×10¹⁴ colony forming units (cfu) per seed, including about 1×10³ cfu/seed, or about 1×10⁴ cfu/seed, 1×10⁵ cfu/seed, or about 1×10⁶ cfu/seed, or about 1×10⁷ cfu/seed, or about 1×10⁸ cfu/seed, or about 1×10⁹ cfu/seed, or about 1×10¹⁰ cfu/seed, or about 1×10¹¹ cfu/seed, or about 1×10¹² cfu/seed, or about 1×10¹³ cfu/seed including about 1×10³ to 1×10⁸ cfu/seed about 1×10³ to 1×10⁷ cfu/seed, about 1×10³ to 1×10⁵ cfu/seed, about 1×10³ to 1×10⁶ cfu/seed, about 1×10³ to 1×10⁴ du/seed, about 1×10³ to 1×10⁹ du/seed, about 1×10³ to 1×10¹⁰ du/seed, about 1×10³ to 1×10¹¹ cfu/seed, about 1×10³ to 1×10¹² cfu/seed, about 1×10³ to 1×10¹³ cfu/seed, about 1×10⁴ to 1×10⁸ cfu/seed about 1×10⁴ to 1×10⁷ cfu/seed, about 1×10⁴ to 1×10⁵ cfu/seed, about 1×10⁴ to 1×10⁶ cfu/seed, about 1×10⁴ to 1×10⁹ cfu/seed, about 1×10⁴ to 1×10¹⁰ cfu/seed, about 1×10¹¹ to 1×10⁹ cfu/seed, about 1×10⁴ to 1×10¹² cfu/seed about 1×10⁴ to 1×10¹³ du/seed, about 1×10⁵ to 1×10⁷ cfu/per seed, about 1×10⁵ to 1×10⁶ cfu/per seed, about 1×10⁵ to 1×10⁸ cfu/per seed, about 1×10⁵ to 1×10⁹ cfu/per seed, about 1×10⁵ to 1×10¹⁰ cfu/per seed, to 1×10⁵ to 1×10¹¹ cfu/per seed, about 1×10⁵ to 1×10¹² cfu/per seed, about 1×10⁵ to 1×10¹³ cfu/per seed, about 1×10⁶ to 1×10⁸ cfu/per seed, about 1×10⁶ to 1×10⁷ cfu/per seed, about 1×10⁶ to 1×10⁹ cfu/per seed, about 1×10⁶ to 1×10¹⁰ cfu/per seed, about 1×10⁶ to 1×10¹¹ cfu/per seed, about 1×10⁶ to 1×10¹² cfu/per seed, about 1×10⁶ to 1×10¹³ cfu/per seed, about 1×10⁷ to 1×10⁸ cfu/per seed, about 1×10⁷ to 1×10⁹ clipper seed, about 1×10⁷ to 1×10¹⁰ cfu/per seed, about 1×10⁷ to 1×10¹¹ cfu/per seed, about 1×10⁷ to 1×10¹² cfu/per seed, about 1×10⁷ to 1×10¹³ cfu/per seed, about 1×10⁸ to 1×10⁹ cfu/per seed, about 1×10⁸ to 1×10¹⁰ cfu/per seed, about 1×10⁸ to 1×10¹¹ cfu/per seed, about 1×10⁸ to 1×10¹² cfu/per seed, about 1×10⁸ to 1×10¹³ cfu/per seed, about 1×10⁹ to 1×10¹⁰ cfu/per seed, about 1×10⁹ to 1×10¹¹ cfu/per seed, about 1×10⁹ to 1×10¹² cfu/per seed, about 1×10⁹ to 1×10¹³ cfu/per seed, about 1×10¹⁰ to 1×10¹¹ cfu/per seed, about 1×10¹¹ to 1×10¹² cfu/per seed, about 1×10¹⁰ to 1×10¹³ cfu/per seed, about 1×10¹¹ to 1×10¹² cfu/per seed, about 1×10¹¹ to 1×10¹³ cfu/per seed, and about 1×10¹² to 1×10¹³ cfu/per seed. As used herein, the tem “colony forming unit” or “cfu” is a unit capable of growing and producing a colony of a microbial strain in favorable conditions. The cfu count serves as an estimate of the number of viable structures or cells in a sample. In some embodiments, concentrations are those of from about 1 to about 100 mg dry bacterial mass per milliliter of carrier (liquid composition) or per gram of carrier (dry formulation). In some embodiments, the concentrations range from 1×10² to about 1×10¹⁰ cell/mL, such as the concentrations ranging from 1×10⁵ to 1×10⁹ cell/mL of the composition or carrier.

In some embodiments, the amount of one or more of the microorganisms in the compositions may vary depending on the final formulation as well as size or type of the plant or seed utilized. Preferably, the one or more microorganisms in the compositions are present in about 0.01% w/w to about 80% w/w of the entire formulation. In some embodiments, the dry weights of one or more microorganisms employed in the compositions is about 0.01%, 0.1%, 1%, 5% w/w to about 65% w/w and most preferably about 1% w/w to about 60% w/w by weight of the entire formulation.

The microbiological compositions may be applied to the target plant (or part(s) thereof) using a variety of conventional methods such as dusting, coating, injecting, rubbing, rolling, dipping, spraying, or brushing, or any other appropriate technique which does not significantly injure the target plant to be treated. Exemplary methods include, but are not limited to, the inoculation of growth medium or soil with suspensions of microbial cells and the coating of plant seeds with microbial cells and/or spores.

Also provided are methods of treating a plant by application of any of a variety of customary formulations in an effective amount to either the soil (i.e., in-furrow), a portion of the plant (i.e., drench) or on the seed before planting (i.e., seed coating or dressing). Customary formulations include solutions, emulsifiable concentrate, wettable powders, suspension concentrate, soluble powders, granules, suspension-emulsion concentrate, natural and synthetic materials impregnated with active compound, and very fine control release capsules in polymeric substances. In certain embodiments, the microbial compositions are formulated in powders that are available in either a ready-to-use formulation or are mixed together at the time of use. In either embodiment, the powder may be admixed with the soil prior to or at the time of planting. In an alternative embodiment, one or both of either the plant growth-promoting agent or biocontrol agent is a liquid formulation that is mixed together at the time of treating. One of ordinary skill in the art understands that an effective amount of the described compositions depends on the final formulation of the composition as well as the size of the plant or the size of the seed to be treated.

Depending on the final formulation and method of application, one or more suitable seed additives (additives) can also be introduced to the compositions. Adhesives such as carboxymethylcellulose and natural and synthetic polymers in the form of powders, granules or latexes, such as gum arabic, chitin, polyvinyl alcohol and polyvinyl acetate, as well as natural phospholipids, such as cephalins and lecithins, and synthetic phospholipids, trehalose, mannitol, sorbitol, myo-inositol, sophorose, maltotriose, glucose, (+)-galactose, methyl-beta-D-galactopyranoside, safener, a lipo-chitooligosaccharide, a triglucosamine lipoglycine salt, an isoflavone, and a ryanodine receptor modulator may be added to the present compositions.

In some embodiments, the compositions are formulated n a single, stable solution, or emulsion, or suspension. For solutions, the active chemical compounds are typically dissolved in solvents before the biological agent is added. Suitable liquid solvents include petroleum based aromatics, such as xylene, toluene or alkylnaphthalenes, aliphatic hydrocarbons, such as cyclohexane or paraffins, for example petroleum fractions, mineral and vegetable oils, alcohols, such as butanol or glycol as well as their ethers and esters, ketones, such as methyl ethyl ketone, methyl isobutyl ketone or cyclohexanone, strongly polar solvents, such as dimethylformamide and dimethyl sulphoxide. For emulsion or suspension, the liquid medium is water. In one embodiment, the chemical agent and biological agent are suspended in separate liquids and mixed at the time of application. In a preferred embodiment of suspension, the chemical agent and biological agent are combined in a ready-to-use formulation that exhibits a reasonably long shelf-life. In use, the liquid can be sprayed or can be applied foliarly as an atomized spray or in-furrow at the time of planting the crop. The liquid composition can be introduced in an effective amount on the seed (i.e., seed coating or dressing) or to the soil (i.e., in-furrow) before germination of the seed or directly to the soil in contact with the roots by utilizing a variety of techniques known in the art including, but not limited to, drip irrigation, sprinklers, soil injection or soil drenching. Optionally, stabilizers and buffers can be added, including alkaline and alkaline earth metal salts and organic acids, such as citric acid and ascorbic acid, inorganic acids, such as hydrochloric acid or sulfuric acid. Biocides can also be added and can include formaldehydes or formaldehyde-releasing agents and derivatives of benzoic acid, such as p-hydroxybenzoic acid.

Seed Coating Formulations

In one aspect, the microbial strains, cultures and/or compositions described herein are formulated as a seed treatment on a plant seed, wherein the plant seed comprises at least one modified or transgenic grain yield enhancing trait. In some embodiments, plant seeds can be partially, or substantially uniformly coated with one or more layers of the microbial strains, cultures, and/or compositions disclosed herein using conventional methods, including but not limited to mixing, spraying or a combination thereof through the use of treatment application equipment that is specifically designed and manufactured to accurately, safely, and efficiently apply seed treatment products to seeds.

In some embodiments, plant seeds can be coated using a coating technology such as, but not limited to, rotary coaters, drum coaters, fluidized bed techniques, spouted beds, rotary mists or a combination thereof. Liquid seed treatments such as those of the present embodiments can be applied, for example, via either a spinning “atomizer” disk or a spray nozzle which evenly distributes the seed treatment onto the seed as it moves though the spray pattern. In certain embodiments, the seed is then mixed or tumbled for an additional period of time to achieve additional treatment distribution and drying. The seeds can be primed or unprimed before coating with the compositions to increase the uniformity of germination and emergence. In an alternative embodiment, a dry powder formulation can be metered onto the moving seed and allowed to mix until completely distributed.

Other aspects provide plant seeds treated with the subject microbial compositions, wherein the plant seed comprises at least one modified or transgenic grain yield enhancing trait. One embodiment provides the seeds having at least part of the surface area coated with a microbiological composition according to the present embodiments. In one embodiment, the microorganism-treated seeds have a microbial strain or spore concentration or microbial cell concentration from about 1×10² to about 1×10¹⁰ per seed. The seeds may also have more spores or microbial cells per seed. The microbial spores and/or cells can be coated freely onto the seeds or, preferably, they can be formulated in a liquid or solid composition before being coated onto the seeds. For example, a solid composition comprising the microorganisms can be prepared by mixing a solid carrier with a suspension of the spores until the solid carriers are impregnated with the spore or cell suspension. This mixture can then be dried to obtain the desired particles.

In some other embodiments, the microbial compositions contain functional agents capable of protecting the seeds from the harmful effects of selective herbicides such as activated carbon, nutrients (fertilizers), and other agents capable of improving the germination and quality of the products or a combination thereof.

Seed coating methods and compositions that are known in the art can be particularly useful when they are modified by the addition of one of the compositions disclosed herein. Such coating methods and apparatus for their application are disclosed in, for example but not limited to, U.S. Pat. Nos. 5,918,413; 5,554,445; 5,389,399; 4,759,945; and 4,465,017. Seed coating compositions are disclosed, for example, in U.S. Pat. Appl. No. US20100154299, U.S. Pat. Nos. 5,939,356; 5,876,739, 5,849,320; 5,791,084, 5,661,103; 5,580,544, 5,328,942; 4,735,015; 4,634,587; 4,372,080, 4,339,456; and 4,245,432, which are all incorporated herein by reference.

A variety of additives can be added to the seed treatment formulations comprising the compositions disclosed herein. Binders can be added and include those composed preferably of an adhesive polymer that can be natural or synthetic without phytotoxic effect on the seed to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; fats; oils; proteins, including gelatin and zeins; gum arables; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

Any of a variety of colorant additives may be employed, including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. Other additives that can be added include trace nutrients such as salts of iron, manganese, boron, copper, cobalt, nickel, molybdenum and zinc. A polymer or other dust control agent can be applied to retain the treatment on the seed surface.

In some specific embodiments, in addition to the microbial cells or spores, the coating can further comprise a layer of adherent. The adherent should be non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrans; alginates; sugars; molasses; polyvinyl pyrrolidones; polysaccharides; proteins; fats; oils; gum arables; gelatins; syrups; and starches. More examples can be found in, for example, U.S. Pat. No. 7,213,367 and U.S. Pat. Appln. No. US20100189693, incorporated herein by reference.

Various additives, such as adherents, dispersants, surfactants, and nutrient and buffer ingredients, can also be included in the seed treatment formulation. Other seed treatment additives include, but are not limited to, coating agents, wetting agents, buffering agents, and polysaccharides. At least one agriculturally acceptable carrier may be added to the seed treatment formulation such as water, solids or dry powders. The dry powders can be derived from a variety of materials such as calcium carbonate, gypsum, vermiculite, talc, humus, activated charcoal, and various phosphorous compounds.

In some embodiments, the seed coating composition can comprise at least one filler which is an organic or inorganic, natural or synthetic component with which the active components are combined to facilitate its application onto the seed. In certain embodiments, the filler is an inert solid such as clays, natural or synthetic silicates, silica, resins, waxes, solid fertilizers (for example, ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminum or magnesium silicates.

The seed treatment formulation may further include one or more of the following ingredients: other pesticides, including compounds that act only below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; chemical fertilizers; biological fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi. These ingredients may be added as a separate layer on the seed or alternatively, may be added as part of the seed coating composition of the embodiments.

In some embodiments, the amount of the composition or other ingredients used in the seed treatment should not inhibit germination of the seed or cause phytotoxic damage to the seed.

The formulation that is used to treat the seed in the compositions of this application may be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation is about 0.5% to about 99% by weight (w/w), 5%-40% or as otherwise formulated by those skilled in the art.

In some embodiments, other conventional inactive or inert ingredients may be incorporated into the seed treatment formulation. Such inert ingredients include, but are not limited to, conventional sticking agents; dispersing agents such as methylcellulose, for example, serve as combined dispersant/sticking agents for use in seed treatments; polyvinyl alcohol; lecithin, polymeric dispersants (e.g., polyvinylpyrrolidone/vinyl acetate); thickeners (e.g., clay thickeners to improve viscosity and reduce settling of particle suspensions); emulsion stabilizers; surfactants; antifreeze compounds (e.g., urea), dyes, colorants, and the like. Further inert ingredients useful in the embodiments of this application can be found in McCutcheon's, vol. 1, “Emulsifiers and Detergents,” MC Publishing Company, Glen Rock, N.J., U.S.A., 1996. Additional inert ingredients useful in the embodiments of this application can be found in McCutcheon's, vol. 2, “Functional Materials,” MC Publishing Company, Glen Rock, N.J., U.S.A., 1996.

The coating formulations of this application may be applied to seeds by a variety of methods, including, but not limited to, mixing in a container (e.g., a bottle or bag), mechanical application, tumbling, spraying, and immersion. A variety of active or inert material can be used for contacting seeds with the microbial compositions, such as conventional film-coating materials including but not limited to water-based film coating materials such as SEPIRET™ (Seppic, Inc., N.J.) and OPACOAT™ (Berwind Pharm. Services, P.A.)

The amount of a composition according to the embodiments of this application that is used for the treatment of the seed will vary depending upon the type of seed and the type of active ingredients, but the treatment will comprise contacting the seeds with an agriculturally effective amount of the described composition. As discussed herein, an effective amount means that amount of the described composition that is sufficient to affect beneficial or desired results. An effective amount can be administered in one or more administrations.

In addition to the coating layer, the seed may be treated with one or more of the following ingredients: other pesticides including fungicides and herbicides; herbicidal safeners; fertilizers and/or biocontrol agents. These ingredients may be added as a separate layer or alternatively, may be added in the coating layer.

The seed coating formulations of the embodiments of this application may be applied to the seeds using a variety of techniques and machines, such as fluidized bed techniques, the roller mill method, rotostatic seed treaters, and drum coaters. Other methods, such as spouted beds may also be useful. The seeds may be pre-sized before coating. In some embodiments, after coating, the seeds are dried and then transferred to a sizing machine for sizing. Such procedures are known to a skilled artisan.

The microorganism-treated seeds may also be enveloped with a film overcoating to protect the coating. Such overcoatings are known in the art and may be applied using fluidized bed and drum film coating techniques, as well as any other suitable methods known in the art.

In another embodiment, microbial strains, isolates, cultures, and/or compositions of this application can be introduced onto a seed by use of solid matrix priming. For example, a quantity of a described composition can be mixed with a solid matrix material and then the seed can be placed into contact with the solid matrix material for a period to allow the composition to be introduced to the seed. The seed can then optionally be separated from the solid matrix material and stored or used, or the mixture of solid matrix material plus seed can be stored or planted directly. Solid matrix materials which are useful in may include polyacrylamide, starch, clay, silica, alumina, soil, sand, polyurea, polyacrylate, or any other material capable of absorbing or adsorbing the composition for a time and releasing that composition into or onto the seed. It is useful to make sure that the composition and the solid matrix material are compatible with each other. For example, the solid matrix material should be chosen so that it can release the composition at a reasonable rate, for example over a period of minutes, hours, days, or months.

In some embodiments, any plant seed capable of germinating to form a plant may be treated with the compositions contemplated herein. Suitable seeds include, but are not limited to, those of cereals, coffee, cole crops, fiber crops, flowers, fruits, legume, oil crops, trees, tuber crops, vegetables, as well as other plants of the monocotyledonous, and dicotyledonous species. In some embodiments, crop seeds are coated include, but are not limited to, bean, carrot, corn, cotton, grasses, lettuce, peanut, pepper, potato, rapeseed, rice, rye, sorghum, soybean, sugarbeet, sunflower, tobacco, and tomato seeds. In certain embodiments, barley or wheat (spring wheat or winter wheat) seeds are coated with the present compositions.

### Methods for Preparing the Composition

Cultures of the microorganisms may be prepared for use in the compositions of the present application using techniques known in the art, including, but not limited to, standard static drying and liquid fermentation. Growth is commonly effected in a bioreactor. A bioreactor may be any appropriate shape or size for growing the microorganisms (PGPMs). A bioreactor may range in size and scale from 10 mL to liters to cubic meters and may be made of stainless steel or any other appropriate material as known and used in the art. The bioreactor may be a batch type bioreactor, a fed batch type or a continuous-type bioreactor (e.g., a continuous stirred reactor). For example, a bioreactor may be a chemostat as known and used in the art of microbiology for growing and harvesting microorganisms. A bioreactor may be obtained from any commercial supplier (See also Bioreactor System Design, Asenjo & Merchuk, CRC Press, 1995). For small scale operations, a batch bioreactor may be used, for example, to test and develop new processes, and for processes that cannot be converted to continuous operations.

Microorganisms or PGPMs grown in a bioreactor may be suspended or immobilized. Growth in the bioreactor is generally under aerobic conditions at suitable temperatures and pH for growth. Cell growth can be achieved at temperatures between 5 and 40° C., with the preferred temperature being in the range of 15 to 30° C., 15 to 28° C., 20 to 30° C., or 15 to 25° C. The pH of the nutrient medium can vary between 4.0 and 9.0, but the preferred operating range is usually slightly acidic to neutral at pH 4.0 to 7.0, or 4.5 to 6.5, or pH 5.0 to 6.0. Typically, maximal cell yield is obtained in 18-96 hours after inoculation.

Optimal conditions for the cultivation of the microorganisms of this application may depend upon the particular strain. However, by virtue of the conditions applied in the selection process and general requirements of most microorganisms, a person of ordinary skill in the art would be able to determine essential nutrients and conditions. The microorganisms or PGPMs would typically be grown in aerobic liquid cultures on media which contain sources of carbon, nitrogen, and inorganic salts that can be assimilated by the microorganism and supportive of efficient cell growth. Exemplary (but not limiting) carbon sources are hexoses such as glucose, but other sources that are readily assimilated such as amino acids, may be substituted. Many inorganic and proteinaceous materials may be used as nitrogen sources in the growth process. Exemplary (but not limiting) nitrogen sources are amino acids and urea but others include gaseous ammonia, inorganic salts of nitrate and ammonium, vitamins, purines, pyrimidines, yeast extract, beef extract, proteose peptone, soybean meal, hydrolysates of casein, distiller's solubles, and the like. Among the inorganic minerals that can be incorporated into the nutrient medium are the customary salts capable of yielding calcium, zinc, iron, manganese, magnesium, copper, cobalt, potassium, sodium, molybdate, phosphate, sulfate, chloride, borate, and like ions. In some embodiments, potato dextrose liquid medium for fungal strains and R2A broth premix for bacterial strains is used.

Methods for Using the Microbial Strains, Cultures, and/or Compositions

Other aspects provide a method for treating a plant seed, comprising a step of exposing or contacting a plant or plant seed with a microbial strain, isolate, culture, and/or composition as described herein, wherein the plant or plant seed comprises at least one modified or transgenic grain yield enhancing trait.

Other aspects provide a method for enhancing the growth or yield of a plant, said method comprising applying an effective amount of a microbial strain, isolate, culture, and/or composition as described herein to the plant or to the plant's surroundings, wherein the plant comprises at least one modified or transgenic grain yield enhancing trait. Another aspect, provides a method for preventing, inhibiting or treating the development of a pathogenic disease of a plant, said method comprising applying an effective amount of a microbial strain, isolate, culture and/or composition as described herein to the plant or to the plant's surroundings, wherein the plant comprises at least one modified or transgenic grain yield enhancing trait. In some embodiments of the methods, the microbial strain is grown in a growth medium or soil of a host plant prior to or concurrent with the host plant growth in said growth medium or soil. In some embodiments, the microbial strain is established as an endophyte on said plant. In some embodiments of the above method, a microbial strain (PGPM) is applied to the plant (or a part thereof) or to the plant's surroundings (e.g., immediate soil layer or rhizosphere) in a culture or a composition at a concentration that is at least 2×, 5×, 10×, 100×, 500×, or 1000× the concentration of the same microbial strain found in nature or detected in an untreated control plant (or a part thereof) or the control plant's surroundings, respectively. In some embodiments, upon or after application, the concentration of the microbial strain (PGPM) in the treated plant (or a part thereof) or the plant's surroundings (e.g., immediate soil layer or rhizosphere) is at least 2×, 5×, 10×, 100×, 500×, or 1000× the concentration of the same microbial strain found or detected in an untreated control plant (or a part thereof) or the control plant's surroundings. In some embodiments of the above method, a microbial strain (PGPM) is applied to the plant (or a part thereof) or to the plant's surroundings (e.g., immediate soil layer or rhizosphere) in a culture or a composition at a concentration of at least 1×10² CFU/mL. In some embodiments, concentration ranges from about 1×10² to about 1×10¹⁰ CFU/mL, such as the concentrations ranging from 1×10⁵ to 1×10⁹ CFU/mL. In some embodiments, application of a microbial strain (PGPM) to the plant (or a part thereof) or to the plant's surroundings (e.g., immediate soil layer or rhizosphere) in a culture or a composition at a concentration that is at least 1×10⁶ CFU/mL leads to a concentration of the microbial strain in the treated plant, plant part or the plant's surroundings that is at least 2× the amount of the strain found in the untreated plant or its surroundings.

In some embodiments of the above method, the microbial strain is established as an endophyte on the plant and the seed offspring of the plant after application. In some embodiments of this aspect, the microbial endophyte introduced into the plant may be an endophytic microorganism having a plant growth-promoting activity, a biological control activity, or a combination of both activities. A variety of methods previously found effective for the introduction of a microbial endophyte into cereal grass species are known in the art. Examples of such methods include those described in U.S. Pat. Appl. No. 20030195117A1, U.S. Pat. Appl. No. 20010032343A1, and U.S. Pat. No. 7,084,331. In some embodiments, the microbial strain, isolate, culture, and/or composition is applied to one or more places selected from the soil, a seed, a root, a flower, a leaf, a fruit, a portion of the plant or the whole plant. In this aspect, the microbial strain, culture or composition may be delivered to the plant by any of the delivery system described herein.

Examples of phytopathogenic diseases that are suitable for applications of the methods and materials include, but are not limited to, diseases caused by a broad range of pathogenic fungi. The methods of the present embodiments are preferably applied against pathogenic fungi that are important or interesting for agriculture, horticulture, plant biomass for the production of biofuel molecules and other chemicals, and/or forestry. In some embodiments, the pathogenic fungi are pathogenic Pseudomonas species (e.g., Pseudomonas solanacearum), Xylella fastidiosa; Ralstonia solanacearum, Xanthomonas campestris, Erwinia amylovora, Fusarium species, Phytophthora species (e.g., P. infestans), Botrytis species, Leptosphaeria species, powdery mildews (Ascomycota) and rusts (Basidiomycota), etc.

Non-limiting examples of plant pathogens of interest include, for instance, Acremonium strictum, Agrobacterium tumefaciens, Alternaria alternata, Alternaria solani, Aphanomyces euteiches, Aspergillus fumigatus, Athelia rolfsii, Aureobasidium pullulans, Bipolaris zeicola, Botrytis cinerea, Calonectria kyotensis, Cephalosporium maydis, Cercospora medicaginis, Cercospora sojina, Colletotrichum coccodes, Colletotrichum fragariae, Colletotrichum graminicola, Coniella diplodiella, Coprinopsis psychromorbida, Corynespora cassiicola, Curvularia pallescens, Cylindrocladium crotalariae, Diplocarpon earlianum, Diplodia gossyina, Diplodia spp., Epicoccum nigrum, Erysiphe dehor acearum, Fusarium graminearum, Fusarium oxysporum, Fusarium oxysporum f.sp. tuberosi, Fusarium proliferatum var. proliferatum, Fusarium solani, Fusarium verticillioides, Ganoderma boninense, Geotrichum candidum, Glomerella tucumanensis, Guignardia bidwellii, Kabatiella zeae, Leptosphaerulina briosiana, Leptotrochila medicaginis, Macrophomina, Macrophomina phaseolina, Magnaporthe grisea, Magnaporthe oryzae, Microsphaera manshurica, Monilinia fructicola, Mycosphaerella fijiensis, Mycosphaerella fragariae, Nigrospora oryzae, Ophiostoma ulmi, Pectobacterium carotovorum, Pellicularia sasakii (Rhizoctonia solani), Peronospora manshurica, Phakopsora pachyrhizi, Phoma foveata, Phoma medicaginis, Phomopsis longicolla, Phytophthora cinnamomi, Phytophthora erythroseptica, Phytophthora fragariae, Phytophthora infestans, Phytophthora medicaginis, Phytophthora megasperma, Phytophthora palmivora, Podosphaera leucotricha, Pseudopeziza medicaginis, Puccinia graminis subsp. Tritici (UG99), Puccinia sorghi, Pyricularia grisea, Pyricularia oryzae, Pythium ultimum, Pythium aphanidermatum, Rhizoctonia solani, Rhizoctonia zeae, Rosellinia sp., Sclerotinia sclerotiorum, Sclerotinina trifoliorum, Sclerotium rolfsii, Septoria glycines, Septoria lycopersici, Setomelanomma turcica, Sphaerotheca macularis, Spongospora subterranea, Stemphylium sp, Synchytrium endobioticum, Thecaphora (Angiosorus), Thielaviopsis, Tilletia indica, Trichoderma viride, Ustilago maydis, Verticillium albo-atrum, Verticillium dahliae, Verticillium dahliae, Xanthomonas axonopodis, or Xanthomonas oryzae pv. oryzae.

In some embodiments, the methods and materials are useful in suppressing the development of the pathogens Aspergillus fumigatus, Botrytis cinerea, Cerpospora betae, Colletotrichum sp., Curvularia spp., Fusarium sp., Ganoderma boninense, Geotrichum candidum, Gibberella sp., Monographella sp., Mycosphaerella fijiensis, Phytophthora palmivora, Phytophthora ramorum, Penicillium sp., Pythium ultimum, Pythium aphanidermatum, Rhizoctonia solani, Rhizopus spp., Schizophyllum spp., Sclerotinia sclerotiorum, Stagnospora sp., Verticillium dahliae, or Xanthomonas axonopodis. In some embodiments, the methods and materials may be used to suppress the development of several plant pathogens of commercial importance, including Fusarium graminearum NRRL-5883, Monographella nivalis ATCC MYA-3968, Gibberella zeae ATCC-16106, Stagnospora nodurum ATCC-26369, Colletotrichum graminicola ATCC-34167, and Penicillium sp. pathogens.

In some embodiments, the method for enhancing the growth or yield of a plant, including any of such methods descried herein, further comprises a step of processing soil before planting a plant, a plant seed or a plant seedling in said soil wherein the plant, plant seed, or plant seedling comprises at least one modified or transgenic grain yield enhancing trait. In some embodiments, the soil is fully or partially sterilized in the soil processing step. In some embodiments, the soil processing method comprises making a microwave radiator move into soil, and thereafter radiating microwaves from the microwave radiator to soil to be processed. Examples of such a method can be found, e.g., in US20060283364. In some embodiments, the soil is fully or partially sterilized by autoclaving (e.g., at 121° C., 1 h or other similar conditions) or by gamma (γ)-irradiation (50 kGy). In some embodiments, the soil is fully or partially sterilized by heating, steaming or gassing with ethylene oxide. In some embodiments, the soil is partially or fully sterilized by soil solarization. Soil solarization is an environmentally friendly method of using solar power for soil processing (e.g., sterilization) by mulching the soil and covering it with tarp, usually with a plastic (e.g. transparent polyethylene) cover, to trap solar energy. Other suitable soil processing methods are known to those skilled in the art.

In some embodiments, the method for enhancing the growth or yield of a plant comprises (a) processing the soil before planting the plant, plant seed or seedling thereof in said soil; (b) planting the plant, plant seed or seedling thereof in the soil processed in step (a); and (3) applying an effective amount of a microbial strain, isolate, culture, and/or composition as described herein to the plant, plant seed or seedling, or surroundings thereof. In some embodiments, the soil is fully sterilized. In some embodiments, the soil is partially sterilized. In some embodiments, the soil is processed by autoclaving in step (a).

### Delivery Systems

Microbial stains, isolates or cultures thereof, or microbial compositions may be delivered through several means. In some embodiments, they are delivered by seed treatment, seed priming, seedling dip, soil application, foliar spray, fruit spray, hive insert, sucker treatment, sett treatment, and a multiple delivery system.

In some embodiments, the microbial strains, cultures thereof or compositions comprising the same, as described herein, may be delivered by direct exposure or contact with a plant, or a plant seed wherein the plant or plant seed comprises at least one modified or transgenic grain yield enhancing trait. In some embodiments, the seed can be coated with a microbial strain (or an isolate or a culture thereof) or a composition thereof. Seed treatment with PGPMs may be effective against several plant diseases.

In some embodiments, the microbial strains, isolates, cultures or compositions, as described herein, can be delivered by direct exposure or contact with a plant seed during seed priming process. Priming with PGPMs may increase germination and improve seedling establishment. Such priming procedures may initiate the physiological process of germination but prevents the emergence of plumule and radicle. It has been recognized that initiation of the physiological process helps in the establishment and proliferation of the PGPMs on the spermosphere.

In some embodiments, the microbial strains, isolates, cultures thereof or compositions comprising the same, as described herein, can be delivered by seedling dip. Plant pathogens often enter host plants through root. In some embodiments, protection of rhizosphere region by prior colonization with PGPMs prevents the establishment of a host-parasite relationship.

In some embodiments, the microbial strains, isolates, cultures or compositions, as described herein, can be delivered by direct application to soil. Soil is the repertoire of both beneficial and pathogenic microbes. In some embodiments, delivering PGPMs to soil can suppress the establishment of pathogenic microbes.

In some embodiments, the microbial strains, isolates, cultures or compositions, as described herein, can be delivered by foliar spray or fruit spray. In some embodiments, delivering PGPMs directly to plant foliage or fruit can suppress pathogenic microbes contributing to various foliar diseases or post-harvest diseases.

In some embodiments, the microbial strains, isolates, cultures or compositions are delivered by hive insert. Honey bees and bumble bees serve as a vector for the dispersal of biocontrol agents of diseases of flowering and fruit crops. In some embodiments, a dispenser can be attached to the hive and loaded with the PGPMs, optionally in combination with other desired agents.

In some embodiments, the microbial strains, isolates, cultures or compositions are delivered by sucker treatment or sett treatment. PGPMs can plant a vital role in the management of soilborne diseases of vegetatively propagated crops. The delivery of PGPMs varies depending upon the crop. For crops such as banana, PGPMs may be delivered through sucker treatment (e.g., sucker dipping). For crops such as sugarcane, PGPMs may be delivered through sett treatment (e.g., sett dipping).

In some embodiments, the microbial strains, isolates, cultures or compositions are delivered by a multiple delivery system comprising two or more of the delivery systems as described herein.

Plant Varieties and Seed Offspring Infected with a Microbial Strain

Also provided, in other aspects of the present embodiments is an artificially infected plant created by artificially introducing a microbial strain disclosed herein to the plant. In some embodiments of this aspect, the microbial strain introduced to the plant may be an endophytic microorganism having a plant growth-promoting activity, a biological control activity, or a combination of both activities. In some embodiments, the microbial strain is established as an endophyte in the plant or a progeny thereof (e.g., the seed offspring) that is exposed to or treated with a microbial (endophytic) strain, isolate, culture or composition thereof as described herein. Accordingly, another embodiment provides a seed of the artificially infected plant, comprising the microbial endophyte disclosed herein.

A variety of methods previously found effective for the introduction of a microbial endophyte into cereal grass species are known in the art. Examples of such methods include those described in U.S. Pat. Appl. No. 20030195117A1, U.S. Pat. Appl. No. 20010032343A1, and U.S. Pat. No. 7,084,331, among others.

In some embodiments, after artificial infection, a DNA sequence of the isolated endophytic microorganism is amplified by PCR and the endophyte is confirmed by carrying out a homology search for the DNA sequence amplified. In some embodiments, a foreign gene that expresses an identifiable means is introduced into the above-mentioned endophytic microorganism, and the presence of the colonization of the above-mentioned endophytic microorganism infecting the plant is confirmed by the above-identifiable means using the foreign gene.

### Suitable Plants

In principle, the methods and compositions of this application may be deployed for any plant species wherein the plant comprises at least one modified or transgenic grain yield enhancing trait. Monocotyledonous as well as dicotyledonous plant species are particularly suitable. The methods and compositions are preferably used with plants that are important or interesting for agriculture, horticulture, for the production of biomass used in producing liquid fuel molecules and other chemicals, and/or forestry.

In still another embodiment, the PGPM compositions, consortia and methods disclosed herein can be used to treat transgenic seed. A transgenic seed refers to the seed of plants containing at least one heterologous gene that allows the expression of a polypeptide or protein not naturally found in the plant. The heterologous gene in transgenic seed can originate, for example, from microorganisms of the species Bacillus, Rhizobium, Pseudomonas, Serratia, Trichoderma, Clavibacter, Glomus or Gliocladium.

Thus, embodiments of this application have use over a broad range of plants, preferably higher plants pertaining to the classes of Angiospermae and Gymnospermae. Plants of the subclasses of the Dicotylodenae and the Monocotyledonae are particularly suitable. Dicotyledonous plants belong to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

The methods and compositions may be used in plants that are important or interesting for agriculture, horticulture, biomass for the production of biofuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza saliva (rice), Helianthus annuus (sunflower), Medicago saliva (alfalfa), Beta vulgaris (sugarbeet), Pennisetum glaucum (pearl millet), Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus), Triticosecale spp. (triticum—wheat X rye), Bambuseae (Bamboo), Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Agrostis spp. (bentgrass), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers. Of interest are plants grown for energy production, so called energy crops, such as cellulose-based energy crops like Panicum virgatum (switchgrass), Sorghum bicolor (sorghum, sudangrass), Miscanthus giganteus (miscanthus), Saccharum sp. (energycane), Populus balsamifera (poplar), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale spp. (triticum—wheat X rye), and Bambuseae (Bamboo); and starch-based energy crops like Zea mays (corn) and Manihot esculenta (cassava); and sugar-based energy crops like Saccharum sp. (sugarcane), Beta vulgaris (sugarbeet), and Sorghum bicolor (L.) Moench (sweet sorghum); and biofuel-producing energy crops like Glycine max (soybean), Brassica napus (canola), Helianthus annuus (sunflower), Carthamus tinctorius (safflower), Jatropha curcas (Jatropha), Ricinus communis (castor), Elaeis guineensis (African oil palm), Elaeis oleifera (American oil palm), Cocos nucifera (coconut), Camelina sativa (wild flax), Pongamia pinnata (Pongam), Olea europaea (olive), Linum usitatissimum (flax), Crambe abyssinica (Abyssinian-kale), and Brassica juncea.

In some embodiments, the methods and compositions may be used in corn, including but not limited to, flour corn (Zea mays var. amylacea), popcorn (Zea mays var. everta), dent corn (Zea mays var. indentata), flint corn (Zea mays var. indurate), sweet corn (Zea mays var. saccharata and Zea mays var. rugosa), waxy corn (Zea mays var. ceratina), amylomaize (Zea mays), pod corn (Zea mays var. tunicata Larrañaga ex A. St. Hil.), and striped maize (Zea mays var. japonica). In some embodiments, the methods and compositions are used in sweetcorn.

This disclosure will be better understood from the Examples which follow. However, one skilled in the art will readily appreciate that the specific methods and results discussed are merely illustrative of the disclosure as described more fully in the embodiments.

### EXAMPLES

### Example 1 Collection of Soil Samples and Sequencing of Soil Microorganisms

Soil samples were collected from agricultural fields. For instance, soil samples were collected from corn fields in the United States. The present application contemplates PGPMs identified and isolated from any suitable types of environmental materials, such as samples collected from, without limitation, soil, rock, plants, animals, organic debris, water, aerosols, etc. From each field V4-V10 stage corn plants were selected, removed from the ground and soil collected. For each plant, height and weight were recorded, soil attached to the roots was collected for cultivation and DNA extraction, and bulk soil surrounding the root structure was collected for soil chemistry analysis and archiving. Non-plant associated soil samples were also collected from multiple locations in the field for baseline soil chemistry analysis and DNA extractions.

Root associated soil samples (about 0.5 g) were collected in triplicate from the rhizosphere of corn plants for DNA extraction and sequencing. Samples were placed into 2-mL screw-cap centrifuge tubes containing a sterile ceramic bead matrix consisting of one 4-mm glass bead (GSM-40), 1.0 g of 1.4- to 1.6-mm zirconium silicate beads (SLZ-15) and 0.75 g of 0.070- to 0.125-mm zirconium silicate beads (BSLZ-1) obtained from Cero Glass (Columbia, Tenn.). Samples were kept cool and transported to the laboratory for DNA extraction.

Samples were mechanically lysed using a FastPrep FP 120 instrument (Bio-101, Vista, Calif.) at 6.5 m/s for 45 s in 1 ml phosphate buffer (200 mM sodium phosphate, 200 mM NaCl, 20 mM EDTA, pH 8.0) and 10% SDS (sodium dodecyl sulfate). Lysed samples were centrifuged at 13,000×g for 5 min at 4° C. to separate the supernatant with DNA and particulate matter. Supernatants were transferred into new 1,5-mL centrifuge tubes and further purified by adding 500 μl phenol-chloroform-isoamyl alcohol (25:24:1) and centrifuging at 13,200×g for 5 min at room temperature. The separated aqueous phase containing the DNA was collected for final purification on QIAprep Plasmid Spin columns (Qiagen, Valencia, Calif.) following manufacturer's instructions.

Identification of key organisms was performed by first extracting genomic DNA and then using 16S rRNA next generation sequencing (NGS) to generate environmental microbial profiles from agricultural fields following the methods of Patin et al. (Microb. Ecol. 65:709-719, 2013). Correlation analysis of microbe 16S sequence tags and desired target phenotypes, included but not limited to, grain yield, plant biomass, plant height, drought tolerance score, and anthesis to silking interval determined the organisms of interest.

### Example 2 Identification of Microbial Consortia

The corn plants for sampling were at the V4-V10 stage of development and were chosen based upon being under or over-performing plants based on visual inspection and comparison with neighboring plants. Plants were collected in trios, that each included an under, over, and average-performing plant that were located in one experimental plot. Under-performing plants were chosen based upon being smaller in size with the average size of plants across the general area or entire field. Over-performing plants were chosen based upon being greater in size than the average size of plants across the general area or entire field. Another criterion for choosing an over-performing plant was that its immediate neighbors were also over-performing relative to the size of plants in the general area or entire field. One average size plant was also chosen per plot. The two middle rows of each plot were left undisturbed to harvest grain yield data at the end of the flowering stress managed experiment.

Prior to sampling, the height of each plant was determined by extending the upper leaves vertically to the highest point and measuring this level. The weight of the plant was determined post-sampling by removing the entire above soil portion of the plant and transferring into a sealed Ziploc quart size bag. The sample was then transferred to the laboratory to determine dry mass.

Corn root-associated soil samples were conducted by digging up the corn plants with a shovel and carefully excavating roots with a sterile stainless-steel spatula. Soil clinging to the roots was removed directly into 2 ml centrifuge tubes containing beads for cell lysis and DNA extraction and profiling were performed as described in Example 1 (See Patin et al. Microb. Ecol. 65:709-719, 2013).

To compare microbial communities associated with corn roots from plants from different fields, the heights and weights of each plant collected from the same field were normalized. A number of different normalization methods were deployed that included Z-scores, interpolation of the values between 0-1 and percent rank. The reason for normalizing the values was to enable comparison of plants between fields that, in some cases, were of different sizes because of different plant genetics, planting dates, soil types, weather, etc.

Approximately 100,000 or more V5V6 16S rRNA sequence tags were generated from each sample. Pearson correlation values were determined for the percent abundance of each 16S rRNA sequence tag and the normalized corn plant weight, height, yield or other parameters of interest across more than 250 microbial profiles from fields in Woodland, Calif. comprising of field corn under managed drought stress conditions. Bacterial 16S rRNA sequence tags with the highest correlation to several parameters of interest were identified. The 16S rRNA sequence tags with the highest correlation to plant performance (grain yield and, drought tolerance) were of primary importance. Secondary consideration was given to 16S tags that negatively correlated with thermal time to reproductive maturity (GDU to Shed, GDU to Silk, GDU Anthesis to Silk Interval). V5V6 16S rRNA sequence tags identified include SEQ ID NOs: 1-474.

Cultivation screens were also performed from the same samples where the root-associated microbial communities were resolved by 16S rRNA gene profiling. Approximately 25,000 isolates were recovered by cultivating on seven different solid medium formulations. The identity of the isolates was determined by PCR-amplifying a portion of the 16S rRNA gene comprising the v1-v9 variable regions. The sequences were trimmed to the same V5V6 region as used for the 16S rRNA gene profiles conducted above. This step allowed for cross indexing between the cultivation and 16S rRNA gene profiling data.

Cultivated strains corresponding to the best plant performance correlated sequence tags and were recovered and advanced for testing their ability to increase predicted plant performance under drought conditions. 16S rRNA sequence tags for the selected strains include SEQ ID NOs: 468-474.

### Example 3 Field Validation of Microbial Yield Enhancement

Field experiments were performed by combining microbial candidates selected for association with increased plant performance under water limited conditions and the AG099 transgenic trait for increased yield and yield stability under drought stress.

The microbial treatments included one consortium (S2695 Arthrobacter globiformis, NRRL Deposit No. B-67444 (v1v9 SEQ ID NO: 462 and v5v6 SEQ ID NO: 469); S2834 Streptomyces vastus, NRRL Deposit No. B-67441(v1v8 SEQ ID NO:463 and v5v6 SEQ ID NO: 469); and S2876 Niastella yeongjuensis, NRRL Deposit No. B-67448(v1v9 SEQ ID NO: 465 and v5v6 SEQ ID NO: 468)) and one single strain (S2695 Arthrobacter globiformis, NRRL Deposit No. B-67444 (v1v9 SEQ ID NO: 462 and v5v6 SEQ ID NO: 174)) applied as seed coatings using a xanthan gum polymer on a set of two hybrids. One hybrid contained a transgene (AG099, a Maize MADS box ZmM28 gene expressing an amino acid sequence of SEQ ID NO: 476) and one hybrid served as a wild type comparison. Irrigation application was managed to impose drought stress during grain-filling at one site, and a second site received standard irrigation to avoid stress. In all locations, the crop was managed according to local commercial practices with effective control of weeds and pests. Yield data were collected in all locations. To evaluate the yield data, a mixed model framework was used to perform the single and multi-location analysis. In the single location analysis, main effect of construct is considered as a fixed effect. However, construct effect may be considered as random in other circumstances. The main effect of event was considered as random. The blocking factors such as replicates and incomplete block within replicates were considered as random. In the multi-location analysis, the main effect of event or construct and its interaction with loc id were considered as random effects. Yield analysis was by ASREML (VSN International Ltd) (Best Linear Unbiased Prediction) (Cullis, B. R et al (1998) Biometrics 54: 1-18, Gilmour, A. R. et al (2009). ASReml User Guide 3.0, Gilmour, A. R., et al (1995) Biometrics 51: 1440-50).

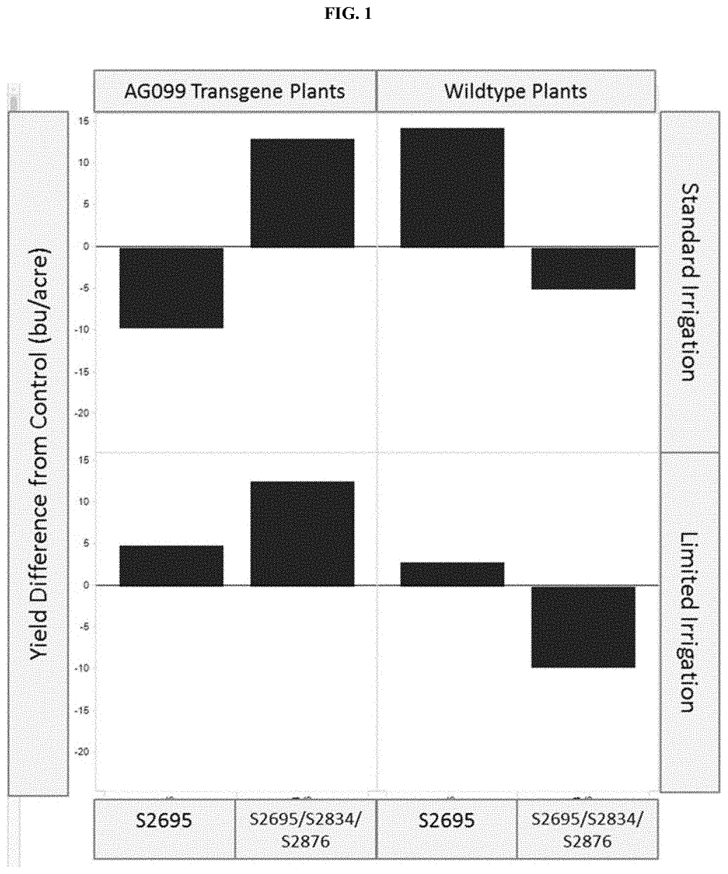

Results from this experiment showed a positive impact on AG099-yield with the consortium of three microbes: S2695 Arthrobacter globiformis, NRRL Deposit No. B-67444; S2834 Streptomyces roseiscleroticus, NRRL Deposit No. B-67441; S2876 Niastella yeongjuensis, NRRL Deposit No. B-67448 relative to wild type genetics (see FIG. 1). WT genetics performed significantly better with the single strain treatment. These differences were statistically significant (P<0.1) only in the location with full irrigation, but a similar trend was observed in the stress location as well. The consortium increased plant height in the AG099 hybrid, but decreased height in the WT as tested.

1. A composition comprising one or more microbial strains wherein the 16S sequence of the one or more microbial strains comprises at least 97% sequence identity to any one of SEQ ID NOs: 165-474 and a plant or plant seed, wherein the plant or plant seed comprises at least one introduced or modified grain yield enhancing trait.

2. The composition of claim 1, further comprising at least one additional microbial strain wherein the 16S sequence of the at least one additional microbial strain comprises at least 97% sequence identity to any one of SEQ ID NOs: 1-474.

3. The composition of claim 1, further comprising at least two additional microbial strains wherein the 16S sequence of the at least two additional microbial strains comprises at least 97% sequence identity to any one of SEQ ID NOs: 1-474.

4. The composition of claim 1, further comprising at least three additional microbial strains wherein the 16S sequence of the at least three additional microbial strains comprises at least 97% sequence identity to any one of SEQ ID NOs: 1-474.

5. A composition comprising one or more microbial strains selected from S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof and a plant or plant seed, wherein the plant or plant seed comprises at least one grain yield enhancing trait.

6. The composition of claim 5, further comprising at least one additional microbial strain wherein the at least one additional microbial strain is selected from P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and 52669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof.

7. The composition of claim 5, further comprising at least two additional microbial strains wherein the at least two additional microbial strains are selected from P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof.

8. The composition of claim 5, further comprising at least three additional microbial strains wherein the at least three additional microbial strains are selected from P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof.

9. The composition of claim 1, wherein the plant or plant seed is a genetically modified plant seed or a transgenic plant seed.

10. The composition of claim 1, wherein grain yield enhancing trait comprises increasing expression of a Maize MADS box ZmM28 gene compared to a control plant not comprising the grain yield enhancing trait.

11. The composition of claim 10, wherein Maize MADS box ZmM28 gene encodes a polypeptide having at least 95% sequence identity to SEQ ID NO: 476.

12. The composition of claim 10, wherein Maize MADS box ZmM28 comprises a polynucleotide sequence having at least 95% sequence identity to SEQ ID NO: 475.

13. The composition of claim 1, further comprising an agriculturally effective amount of a compound or composition selected from the group consisting of a nutrient, a fertilizer, an acaricide, a bactericide, a fungicide, an insecticide, a microbicide, a nematicide, and a pesticide.

14. The composition according to any one of claims 1-9, further comprising a carrier.

15. The composition according to claim 11, wherein said carrier is selected from peat, turf, talc, lignite, kaolinite, pyrophyllite, zeolite, montmorillonite, alginate, press mud, sawdust, perlite, mica, silicas, quartz powder, calcium bentonite, vermiculite and mixtures thereof.

16. The composition according to claim 1, wherein the one or more microbial strains are prepared as a formulation selected from the group consisting of an emulsion, a colloid, a dust, a granule, a pellet, a powder, a spray, and a solution.

17. The composition according to claim 1, further comprising a biocontrol agent selected from the group consisting of a bacteria, a fungus, a yeast, a protozoa, a virus, an entomopathogenic nematode, a botanical extract, a protein, a nucleic acid, a secondary metabolite, and an innoculant.

18. The composition according to claim 1, further comprising a compound selected from the group consisting of a safener, a lipo-chitooligosaccharide, a triglucosamine lipoglycine salt, an isoflavone, and a ryanodine receptor modulator.

19. A method of increasing plant grain yield of a plant, the method comprising applying one or more microbial strains to the plant or plant part, wherein the 16S sequence of the one or more microbial strains comprises at least 97% sequence identity to any one of SEQ ID Nos: 1-474, wherein the plant comprises at least one modified grain yield enhancing trait.

20. The method of claim 19, wherein the plant part comprises a seed, and wherein the seed is coated with a seed additive.

21. The method of claim 20, wherein the plant or plant component comprises event DP202216-6 having an ATCC Accession Number of PTA-124653.

22. The method of claim 19, wherein the composition comprises at least 1×10² CFUs of the one or more microbial strains.

23. The method of claim 19, wherein the plant is a genetically modified plant seed or a transgenic plant seed.

24. The method of claim 21, wherein the coating further comprises a biocontrol agent selected from the group consisting of a bacterium, a fungus, a yeast, a protozoan, a virus, an entomopathogenic nematode, a botanical extract, a protein, a nucleic acid, a secondary metabolite, and an innoculant.

25. The method of claim 21, wherein the coating further comprises a compound selected from the group consisting of a safener, a lipo-chitooligosaccharide, a triglucosamine lipoglycine salt, an isoflavone, and a ryanodine receptor modulator.

26. A composition comprising one or more plant growth promoting microbes and a plant or plant seed, wherein the plant or plant seed comprises at least one grain yield enhancing trait, wherein the grain enhancing yield trait comprises a Maize MADS box ZmM28 gene.

27. The composition of claim 26, wherein the one or more plant growth promoting microbes comprises a 16S sequence having at least 97% sequence identity to any one of SEQ ID NOs: 1-474 or any one of P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof.

28. The composition of claim 10, wherein Maize MADS box ZmM28 gene encodes a polypeptide having at least 95% sequence identity to SEQ ID NO: 476.

29. The composition of claim 26, wherein Maize MADS box ZmM28 comprises a polynucleotide sequence having at least 95% sequence identity to SEQ ID NO: 475.

30. The composition of claim 26, further comprising an agriculturally effective amount of a compound or composition selected from the group consisting of a nutrient, a fertilizer, an acaricide, a bactericide, a fungicide, an insecticide, a microbicide, a nematicide, and a pesticide.

31. The composition of claim 26, further comprising a carrier.

32. The composition of claim 31, wherein said carrier is selected from peat, turf, talc, lignite, kaolinite, pyrophyllite, zeolite, montmorillonite, alginate, press mud, sawdust, perlite, mica, silicas, quartz powder, calcium bentonite, vermiculite and mixtures thereof.

33. The composition of claim 26, further comprising a biocontrol agent selected from the group consisting of a bacteria, a fungus, a yeast, a protozoa, a virus, an entomopathogenic nematode, a botanical extract, a protein, a nucleic acid, a secondary metabolite, and an innoculant.

34. The composition of claim 26, further comprising a compound selected from the group consisting of a safener, a lipo-chitooligosaccharide, a triglucosamine lipoglycine salt, an isoflavone, and a ryanodine receptor modulator.

35. A method of increasing plant grain yield of a plant comprising applying one or more plant growth promoting microbes and a plant or plant seed, wherein the plant or plant seed comprises at least one grain yield enhancing trait, wherein the grain enhancing yield trait comprises a Maize MADS box ZmM28 gene.

36. The method of claim 35, wherein the plant comprises a seed.

37. The method of claim 36, further comprising coating the seed is coated with a seed additive.

38. The method of claim 35, wherein the composition comprises at least 1×10² CFUs of the one or more microbial strains.

39. The method of claim 35, wherein the plant is a genetically modified plant seed or a transgenic plant seed.

40. The method of claim 36, wherein the coating further comprises a biocontrol agent selected from the group consisting of a bacterium, a fungus, a yeast, a protozoan, a virus, an entomopathogenic nematode, a botanical extract, a protein, a nucleic acid, a secondary metabolite, and an innoculant.

41. The method of claim 36, wherein the coating further comprises a compound selected from the group consisting of a safener, a lipo-chitooligosaccharide, a triglucosamine lipoglycine salt, an isoflavone, and a ryanodine receptor modulator.

42. A composition comprising one or more plant growth promoting microbes and a plant or plant seed, wherein the plant or plant seed comprises event DP202216-6 having an ATCC Accession Number of PTA-124653.

43. The composition of claim 42, wherein the one or more plant growth promoting microbes comprises a 16S sequence having at least 97% sequence identity to any one of SEQ ID NOs: 1-474 or any one of P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof.

44. A composition of claim 42, further comprising an agriculturally effective amount of a compound or composition selected from the group consisting of a nutrient, a fertilizer, an acaricide, a bactericide, a fungicide, an insecticide, a microbicide, a nematicide, and a pesticide.

45. The composition of claim 42, further comprising a carrier.

46. The composition of claim 45, wherein said carrier is selected from peat, turf, talc, lignite, kaolinite, pyrophyllite, zeolite, montmorillonite, alginate, press mud, sawdust, perlite, mica, silicas, quartz powder, calcium bentonite, vermiculite and mixtures thereof.

47. The composition of claim 42, further comprising a biocontrol agent selected from the group consisting of a bacteria, a fungus, a yeast, a protozoa, a virus, an entomopathogenic nematode, a botanical extract, a protein, a nucleic acid, a secondary metabolite, and an innoculant.

48. The composition of claim 42, further comprising a compound selected from the group consisting of a safener, a lipo-chitooligosaccharide, a triglucosamine lipoglycine salt, an isoflavone, and a ryanodine receptor modulator.

49. A method of increasing maize grain yield, the method comprising applying one or more plant growth promoting microbes to a maize plant or maize seed, wherein the maize plant or the maize seed comprises event DP202216-6 having an ATCC Accession Number of PTA-124653.

50. The composition of claim 49, wherein the one or more plant growth promoting microbes comprises a 16S sequence having at least 97% sequence identity to any one of SEQ ID NOs: 1-474 or any one of P0032_C7, P0048_B9, P0050_F5 (also referred to as S2199), P0035_B2 (also referred to as S2145, NRRL Deposit No. B-67091), P0020_B1, P0047_A1 (also referred to as S2284, NRRL Deposit No. B-67102), P0033_E1 (also referred to as S2177), P0032_A8 (also referred to as S2181, NRRL Deposit No. B-67099), P0049_E7, P0042_A8 (also referred to as S2167), P0042_D5 (also referred to as S2165), P0042_B2 (also referred to as S2168, NRRL Deposit No. B-67096), P0042_B12 (also referred to as S2189), P0042_C2 (also referred to as S2173, NRRL Deposit No. B-67098), P0042_D10 (also referred to as S2172, NRRL Deposit No. B-67097), P0044_A3 (also referred to as S2476), P0018_A11, P0044_A5, P0047_E2, P0047_C1, P0038_D2 or S2166, P0042_E1, P0047_E8, P0018_A1, S2159_P0058_B9 (NRRL Deposit No. B-67092), S2161_P0054_E8 (NRRL Deposit No. B-67094), S2164_P0054_F4, P0057_A3 (also referred to as S2160, NRRL Deposit No. B-67093), S2142_P0061_E11, S2163_P0019_A12 (NRRL Deposit No. B-67095), P0147_D10 (also referred to as S2291, NRRL Deposit No. B-67104), P0147_G10 (also referred to as S2292, NRRL Deposit No. B-67105), P0160_F7 (also referred to as S2351), P0140_C10 (also referred to as S2300, NRRL Deposit No. B-67107), S2387, P0157_G5 (also referred to as S2303, NRRL Deposit No. B-67108), P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106) S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. 67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), and S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRLB Deposit No. 67445), S2837 (NRRL Deposit No. B-67446), S2839 (NRRL Deposit No. B-67447), S2876 (NRRL Deposit No. B-67448), S2871 (NRRL Deposit No. B-67440), S2145-2 (NRRL B-67331), S2292-2 (NRRL B-67332), S2300-2 (NRRL B-67333), S2303-2 (NRRL B-67334), S2375-2 (NRRL B-67335), S2382-2 (NRRL B-67336), S2423-2 (NRRL B-67337), S2669-2 (NRRL B-67338), or a strain derived therefrom, or a culture thereof.

51. The method of claim 49, wherein the plant comprises a seed.

52. The method of claim 51, further comprising coating the seed is coated with a seed additive.

53. The method of claim 49, wherein the composition comprises at least 1×10² CFUs of the one or more microbial strains.

54. The method of claim 51, wherein the coating further comprises a biocontrol agent selected from the group consisting of a bacterium, a fungus, a yeast, a protozoan, a virus, an entomopathogenic nematode, a botanical extract, a protein, a nucleic acid, a secondary metabolite, and an innoculant.

55. The method of claim 49, wherein the coating further comprises a compound selected from the group consisting of a safener, a lipo-chitooligosaccharide, a triglucosamine lipoglycine salt, an isoflavone, and a ryanodine receptor modulator.


SEQUENCE LISTING (476 sequences)

SEQ ID NO: 1 - DNA, 250 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatacgagaagcctctagagatagaggtctctttggacactcgtatacaggtggtgc

SEQ ID NO: 2 - DNA, 586 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatacgagaagcctctagagatagaggtctctttggacactcgtatacaggtggtgcatggctgtcgtcagctcgtgtcgtgagatgttgggttaagtcccgcaacgagcgcaaccctcgtcttatgttgccagcacgtaatggtggggactcataagagactgccggggtcaactcggaggaaggtggggatgacgtcaagtcatcatgccccttatgcctagggcttcacgcatgctacaatggccggtacaaagggctgcgaaatcgcaagatggagcgaatcccaaaaagccggtctcagttcggattggggtctgcaactcgaccccatgaagtcggagtcgctagtaatcgcagatcagcaacgctgcggtgaatacgttcccgggc

SEQ ID NO: 3 - DNA, 586 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatacgagaagcctctagagatagaggtctctttggacactcgtatacaggtggtgcatggctgtcgtcagctcgtgtcgtgagatgttgggttaagtcccgcaacgagcgcaaccctcgtcttatgttgccagcacgtcatggtggggactcataagagactgccggggtcaactcggaggaaggtggggatgacgtcaagtcatcatgccccttatgcctagggcttcacgcatgctacaatggccggtacaaagggctgcgaaatcgcaagatggagcgaatcccaaaaagccggtctcagttcggattggggtctgcaactcgaccccatgaagtcggagtcgctagtaatcgcagatcagcaacgctgcggtgaatacgttcccgggc

SEQ ID NO: 4 - DNA, 250 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatacgagaagcctctagagatagaggtctctttggacacttgtatacaggtggtgc

SEQ ID NO: 5 - DNA, 588 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatacgagaagcctctagagatagaggtctctttggacacttgtatacaggtggtgcatggctgtcgtcagctcgtgtcgtgagatgttgggttaagtcccgcaacgagcgcaaccctcgtcttatgttgccagcgggtaatgccggggactcataagagactgccggggtcaactcggaggaaggtggggatgacgtcaagtcatcatgccccttatgcctagggcttcacgcatgctacaatggccggtacaaagggctgcgaaatcgcaagatggagcgaatcccaaaaagccggtctcagttcggattggggtctgcaactcgaccccatgaagtcggagtcgctagtaatcgcagatcagcaacgctgcggtgaatacgttcccgggcct

SEQ ID NO: 6 - DNA, 94 bp, Unknown
  16S DNA Tag
gtatacaggtggtgcatggctgtcgtcagctcgtgtcgtgagatgttgggttaagtcccgcaacgagcgcaaccctcgtcttatgttgccagcg

SEQ ID NO: 7 - DNA, 588 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatacgagaagcctctagagatagaggtctctttggacacttgtatacaggtggtgcatggctgtcgtcagctcgtgtcgtgagatgttgggttaagtcccgcaacgagcgcaaccctcgtcttatgttgccagcacgtcatggtggggactcataagagactgccggggtcaactcggaggaaggtggggatgacgtcaagtcatcatgccccttatgcctagggcttcacgcatgctacaatggccggtacaaagggctgcgataccgcaaggtggagcgaatcccaaaaagccggtctcagttcggattggggtctgcaactcgaccccatgaagtcggagtcgctagtaatcgcagatcagcaacgctgcggtgaatacgttcccgggcct

SEQ ID NO: 8 - DNA, 245 bp, Unknown
  16S DNA Tag
tagaggtctctttggacacttgtatacaggtggtgcatggctgtcgtcagctcgtgtcgtgagatgttgggttaagtcccgcaacgagcgcaaccctcgtcttatgttgccagcacgtcatggtggggactcataagagactgccggggtcaactcggaggaaggtggggatgacgtcaagtcatcatgccccttatgcctagggcttcacgcatgctacaatggccggtacaaagggctgcgat

SEQ ID NO: 9 - DNA, 250 bp, Unknown
  16S DNA Tag
cacgccctaaacgatgcgaactggatgttgggagcaactaggctctcagtatcgaagctaacgcgttaagttcgccgcctggggagtacggtcgcaagactgaaactcaaaggaattgacgggggcccgcacaagcggtggagtatgtggtttaattcgatgcaacgcgcagaaccttacctggccttgacatccacggaacccttgagagatcgaggggtgccttcgggaaccgtgagacaggtgctgc

SEQ ID NO: 10 - DNA, 588 bp, Unknown
  16S DNA Tag
cacgccctaaacgatgcgaactggatgttgggagcaactaggctctcagtatcgaagctaacgcgttaagttcgccgcctggggagtacggtcgcaagactgaaactcaaaggaattgacgggggcccgcacaagcggtggagtatgtggtttaattcgatgcaacgcgcagaaccttacctggccttgacatccacggaacccttgagagatcgaggggtgccttcgggaaccgtgagacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacggccagggctacacacgtactacaatggtggggacagagggctgcgatcccgcgagggtgagccaatcccagaaaccccatctcagtccggatcggagtctgcaactcgactccgtgaagtcggaatcgctagtaatcgcagatcagcattgctgcggtgaatacgttcccgggcct

SEQ ID NO: 11 - DNA, 252 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggataccttcagttcggctggaccggtgacaggtgctgc

SEQ ID NO: 12 - DNA, 587 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggataccttcagttcggctggaccggtgacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacgttcccgggcct

SEQ ID NO: 13 - DNA, 587 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggataccttcagttcggctggaccggtgacaggtgctgcatggctgtcgtcagctcgtgtcgtgagatgttgggttaagtcccgcaacgagcgcaaccctcgcccttagttgccatcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacgttcccgggcct

SEQ ID NO: 14 - DNA, 249 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgc

SEQ ID NO: 15 - DNA, 586 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggcct

SEQ ID NO: 16 - DNA, 249 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgc

SEQ ID NO: 17 - DNA, 574 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatac

SEQ ID NO: 18 - DNA, 249 bp, Unknown
  16S DNA Tag
cacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgc

SEQ ID NO: 19 - DNA, 585 bp, Unknown
  16S DNA Tag
cacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggcct

SEQ ID NO: 20 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgc

SEQ ID NO: 21 - DNA, 585 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggcaggtacaatgagctgcgaagccgtgaggcggagcgaatctcaaaaagcctgtctcagttcggattggggtctgcaactcgaccccatgaagtcggagttgctagtaatcgcagatcagcattgctgcggtgaatacgtt

SEQ ID NO: 22 - DNA, 585 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaaagagctgcgaagccgtgaggtggagcgaatctcaaaaagccggtctcagttcggattggggtctgcaactcgaccccatgaagtcggagttgctagtaatcgcagatcagcattgctgcggtgaatacgtt

SEQ ID NO: 23 - DNA, 585 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaaagagctgcgaaaccgtgaggtggagcgaatctcaaaaagccggtctcagttcggattggggtctgcaactcgaccccatgaagtcggagttgctagtaatcgcagatcagcattgctgcggtgaatacgtt

SEQ ID NO: 24 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacgccggaaagcatcagagatggtgccccccttgtggtcggtgtacaggtggtgc

SEQ ID NO: 25 - DNA, 594 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacgccggaaagcatcagagatggtgccccccttgtggtcggtgtacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagttgctagtaatcgcagatcagcattgctgcggtgaatacgttcccgggcct

SEQ ID NO: 26 - DNA, 303 bp, Unknown
  16S DNA Tag
caacgcgaagaaccttaccaaggcttgacatacgccggaaagcatcagagatggtgccccccttgtggtcggtgtacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggccggtacaatgagctgcgata

SEQ ID NO: 27 - DNA, 249 bp, Unknown
  16S DNA Tag
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaacgtctggagacaggcgcccccttgtggtcggtgtacaggtggtgc

SEQ ID NO: 28 - DNA, 581 bp, Unknown
  16S DNA Tag
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaacgtctggagacaggcgcccccttgtggtcggtgtacaggtggtgcatggctgtcgtcagctcgtgtcgtgagatgttgggttaagtcccgcaacgagcgcaacccttgtcccgtgttgccagcaagcccttcggggtgttggggactcacgggagaccgccggggtcaactcggaggaggtggggacgacgtcaagtcatcatgccccttatgtcttgggctgcacacgtgctacaatggccggtacaatgagctgcgataccgcaaggtggagcgaatctcaaaaagccggtctcagttcggattggggtctgcaactcgaccccatgaagtcggagtcgctagtaatcgcagatcagcattgctgcggtgaatac

SEQ ID NO: 29 - DNA, 164 bp, Unknown
  16S DNA Tag
gcccccttgtggtcggtgtacaggtggtgcatggctgtcgtcagctcgtgtcgtgagatgttgggttaagtcccgcaacgagcgcaacccttgtcccgtgttgccagcaagcccttcggggtgttggggactcacgggagaccgccggggtcaactcggaggag

SEQ ID NO: 30 - DNA, 250 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaaagacctggaaacaggtgccccgcttgcggtcggtttacaggtggtgc

SEQ ID NO: 31 - DNA, 569 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaaagacctggaaacaggtgccccgcttgcggtcggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcgg

SEQ ID NO: 32 - DNA, 250 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaagacctggaaacaggtcccccacttgtggtcggtttacaggtggtgc

SEQ ID NO: 33 - DNA, 576 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaagacctggaaacaggtcccccacttgtggtcggtttacaggtggtgcatggttgtcgtcagctcgtgtcgtgagatgttgggttaagtcccgcaacgagcgcaaccctcgttccatgttgccagcgcgttatggcggggactcatgggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatac

SEQ ID NO: 34 - DNA, 250 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaagacctggaaacaggtcccccgcttgcggtcggtttacaggtggtgc

SEQ ID NO: 35 - DNA, 588 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggtaagacctggaaacaggtcccccgcttgcggtcggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggcct

SEQ ID NO: 36 - DNA, 249 bp, Unknown
  16S DNA Tag
cacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgc

SEQ ID NO: 37 - DNA, 586 bp, Unknown
  16S DNA Tag
cacgccgtaaacgatgtcgacttggaggttgttcccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgcgtgatggcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggcct

SEQ ID NO: 38 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgc

SEQ ID NO: 39 - DNA, 591 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggcaggtacaatgagctgcgatgccgcgaggcggagcgaatctcaaaaagcctgtctcagttcggattggggtctgcaactcgaccccatgaagtcggagttgctagtaatcgcagatcagcattgctgcggtgaatacgttcccggg

SEQ ID NO: 40 - DNA, 259 bp, Unknown
  16S DNA Tag
accggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggcaggtacaat

SEQ ID NO: 41 - DNA, 249 bp, Unknown
  16S DNA Tag
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaagcattagagatagtgccccccttgtggtcggtgtacaggtggtgc

SEQ ID NO: 42 - DNA, 593 bp, Unknown
  16S DNA Tag
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaagcattagagatagtgccccccttgtggtcggtgtacaggtggtgcatggctgtcgtcagctcgtgtcgtgagatgttgggttaagtcccgcaacgagcgcaacccttgtcccgtgttgccagcaggcccttgtggtgctggggactcacgggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggcct

SEQ ID NO: 43 - DNA, 252 bp, Unknown
  16S DNA Tag
cacgccgtaaactatgagagctagccgtcggcaagtttacttgtcggtggcgcagctaacgcattaagctctccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggaaaccttcagttcggctggaccggtgacaggtgctgc

SEQ ID NO: 44 - DNA, 587 bp, Unknown
  16S DNA Tag
cacgccgtaaactatgagagctagccgtcggcaagtttacttgtcggtggcgcagctaacgcattaagctctccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggaaaccttcagttcggctggaccggtgacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcagatcagcatgctgcggtgaatacgttcccgggcct

SEQ ID NO: 45 - DNA, 263 bp, Unknown
  16S DNA Tag
tccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggaaaccttcagttcggctggaccggtgacaggtgctgcatggctgtcgtcagctcgtgtcgtgagatgttgggttaagtcccgcaacgagcgcaaccctcgcccttagttgccagcattc

SEQ ID NO: 46 - DNA, 250 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaaacgcctggaaacaggtgccccgcttgcggtcggtttacaggtggtgc

SEQ ID NO: 47 - DNA, 588 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaaacgcctggaaacaggtgccccgcttgcggtcggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggcct

SEQ ID NO: 48 - DNA, 290 bp, Unknown
  16S DNA Tag
aaacgcctggaaacaggtgccccgcttgcggtcggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccgg

SEQ ID NO: 49 - DNA, 250 bp, Unknown
  16S DNA Tag
cacgccgtaaacgttgggcgctagatgtggggaccattccacggtttccgtgtcgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatatacgagaacgggccagaaatggtcaactctttggacactcgtaaacaggtggtgc

SEQ ID NO: 50 - DNA, 588 bp, Unknown
  16S DNA Tag
cacgccgtaaacgttgggcgctagatgtggggaccattccacggtttccgtgtcgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcataggagactgccggggtcaactcggaggaaggtggggatgacgtcaaatcatcatgccccttatgtcttgggcttcacgcatgctacaatggccggtacaaagggctgcaataccgtaaggtggagcgaatcccaaaaagccggtctcagttcggattgaggtctgcaactcgacctcatgaagtcggagtcgctagtaatcgcagatcagcaacgctgcggtgaatacgttcccgggcct

SEQ ID NO: 51 - DNA, 248 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatagggaaatctgctagagatagcaggtccgtaagggctctatacaggtggtgc

SEQ ID NO: 52 - DNA, 586 bp, Unknown
  16S DNA Tag
cacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatagggaaatctgctagagatagcaggtccgtaagggctctatacaggtggtgcatggctgtcgtcagctcgtgtcgtgagatgttgggttaagtcccgcaacgagcgcaaccctcgtcttatgttgccagcacgtcatggtggggactcataagagactgccggggtcaactcggaggaaggtggggatgacgtcaagtcatcatgccccttatgcctagggcttcacgcatgctacaatggccggtacaaagggctgcgaaatcgcaagatggagcgaatcccaaaaagccggtctcagttcggattggggtctgcaactcgaccccatgaagtcggagtcgctagtaatcgcagatcagcaacgctgcggtgaatacgttcccgggcct

SEQ ID NO: 53 - DNA, 223 bp, Unknown
  16S DNA Tag
caggtccgtaagggctctatacaggtggtgcatggctgtcgtcagctcgtgtcgtgagatgttgggttaagtcccgcaacgagcgcaaccctcgtcttatgttgccagcacgtcatggtggggactcataagagactgccggggtcaactcggaggaaggtggggatgacgtcaagtcatcatgccccttatgcctagggcttcacgcatgctacaatggccg

SEQ ID NO: 54 - DNA, 252 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagaaatggataccttcagttcggctggaccggtgacaggtgctgc

SEQ ID NO: 55 - DNA, 587 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagaaatggataccttcagttcggctggaccggtgacaggtgctgcatggctgtcgtcagctcgtgtcgtgagatgttgggttaagtcccgcaacgagcgcaaccctcgcccttagttgccatcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacgttcccgggcct

SEQ ID NO: 56 - DNA, 582 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagaaatggataccttcagttcggctggaccggtgacaggtgctgcatggctgtcgtcagctcgtgtcgtgagatgttgggttaagtcccgcaacgagcgcaaccctcgcccttagttgccatcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaaatcgcggatcagcatgccgcggggaatacgttccc

SEQ ID NO: 57 - DNA, 78 bp, Unknown
  16S DNA Tag
cgagctaatctccaaaagccatctcagttcggattgcactctgcaactcgagtgcatgaagttggaatcgctagtaaa

SEQ ID NO: 58 - DNA, 252 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggaaaccttcagttcggctggaccggtgacaggtgctgc

SEQ ID NO: 59 - DNA, 576 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggaaaccttcagttcggctggaccggtgacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacg

SEQ ID NO: 60 - DNA, 252 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggaaatcttcagttcggctggaccggtgacaggtgctgc

SEQ ID NO: 61 - DNA, 577 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggaaatcttcagttcggctggaccggtgacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacgt

SEQ ID NO: 62 - DNA, 249 bp, Unknown
  16S DNA Tag
cacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaacggctagagatagtcgcccccttgtggtcggtatacaggtggtgc

SEQ ID NO: 63 - DNA, 593 bp, Unknown
  16S DNA Tag
cacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaacggctagagatagtcgcccccttgtggtcggtatacaggtggtgcatggttgtcgtcagctcgtgtcgtgagatgttgggttaagtcccgcaacgagcgcaacccttgttctgtgttgccagcatgcctttcggggtgatggggactcacaggagactgccggggtcaactcggaggaaggtggggacgacgtcaaatcatcatgccccttatgtcttgggctgcacacgtgctacaatggtcggtacaaagggctgcaatgccgcgaggcggagcgaatcccaaaaagccggcctcagttcggattggggtctgcaactcgaccccatgaagttggagttgctagtaatcgcagatcagcatgctgcggtgaatacgttcccgggcct

SEQ ID NO: 64 - DNA, 106 bp, Unknown
  16S DNA Tag
agactgccggggtcaactcggaggaaggtggggacgacgtcaaatcatcatgccccttatgtcttgggctgcacacgtgctacaatggtcggtacaaagggctgca

SEQ ID NO: 65 - DNA, 251 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaccgccgcagaaatgtggtttcccctttggggctggtttacaggtggtgc

SEQ ID NO: 66 - DNA, 588 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaccgccgcagaaatgtggtttcccctttggggctggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggcc

SEQ ID NO: 67 - DNA, 251 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaccgccgcagaaatgtggtttcccctttggggctggtttacaggtggtgc

SEQ ID NO: 68 - DNA, 588 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaccgccgcagaaatgtggtttcccctttggggctggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcgggaatacgttcccgggcct

SEQ ID NO: 69 - DNA, 238 bp, Unknown
  16S DNA Tag
gatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggg

SEQ ID NO: 70 - DNA, 251 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaccgctgcagaaatgtggtttcccctttggggctggtttacaggtggtgc

SEQ ID NO: 71 - DNA, 591 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaccgctgcagaaatgtggtttcccctttggggctggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgccggtgaatacgttcccggggcct

SEQ ID NO: 72 - DNA, 94 bp, Unknown
  16S DNA Tag
taatcccaaaaagccggtctcagttcggattggggtctgcaactcgaccccatgaagtcggagtcgctagtaatcgcagatcagcaacgctgcc

SEQ ID NO: 73 - DNA, 589 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaccgctgcagaaatgtggtttcccctttggggctggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggcct

SEQ ID NO: 74 - DNA, 589 bp, Unknown; identical to SEQ ID NO: 73
  16S DNA Tag

SEQ ID NO: 75 - DNA, 250 bp, Unknown
  16S DNA Tag
cacgccctaaacgatgtcaactagttgtcgggtcttcattgacttggtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtatggaaccctgctgagaggtgggggtgcccgaaagggagccataacacaggtgctgc

SEQ ID NO: 76 - DNA, 580 bp, Unknown
  16S DNA Tag
cacgccctaaacgatgtcaactagttgtcgggtcttcattgacttggtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtatggaaccctgctgagaggtgggggtgcccgaaagggagccataacacaggtgctgcatggctgtcgtcagctcgtgtcgtgagatgttgggttaagtcccgcaacgagcgcaacccttgtccctagttgctacgcaagagcactccagggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggatcgcactctgcaactcgagtgcgtgaagctggaatcgctagtaatcgcggatcagcatgccgcggtgaatacgttcccgggtct

SEQ ID NO: 77 - DNA, 594 bp, Unknown
  16S DNA Tag
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaacgtctggagacaggcgcccccttgtggtcggtgtacaggtggtgcatggctgtcgtcagctcgtgtcgtgagatgttgggttaagtcccgcaacgagcgcaacccttgtcccgtgttgccagcaagcccttcggggtgttggggactcacgggagaccgccggggtcaactcggaggaaggtggggacgacgtcaagtcatcatgccccttatgtcttgggctgcacacgtgctacaatggccggtacaatgagctgcgataccgcaaggtggagcgaatctcaaaaagccggtctcagttcggattggggtctgcaactcgaccccatgaagtcggagtcgctagtaatcgcagatcagcattgctgcggtgaatacgttcccgggcct

SEQ ID NO: 78 - DNA, 586 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgtgatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggcct

SEQ ID NO: 79 - DNA, 439 bp, Unknown
  16S DNA Tag
cttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgtgatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccat

SEQ ID NO: 80 - DNA, 586 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggcct

SEQ ID NO: 81 - DNA, 250 bp, Unknown
  16S DNA Tag
cacgccctaaacgatgcgaactggatgttggtctcaactcggagatcagtgtcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtctggaatccctgagagatcggggagtgccttcgggaaccagaacacaggtgctgc

SEQ ID NO: 82 - DNA, 588 bp, Unknown
  16S DNA Tag
cacgccctaaacgatgcgaactggatgttggtctcaactcggagatcagtgtcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtctggaatccctgagagatcggggagtgccttcgggaaccagaacacaggtgctgcatggctgtcgtcagctcgtgtcgtgagatgttgggttaagtcccgcaacgagcgcaacccttgtccttagttgccagcacgtaaaggtgggaactctaaggagactgccggtgacaaaccggaggaaggtggggatgacgtcaagtcatcatggcccttacggccagggctacacacgtactacaatggtcggtacagagggttgcaataccgcgaggtggagccaatcccagaaagccgatcccagtccggatcgaagtctgcaactcgacttcgtgaagtcggaatcgctagtaatcgcggatcagctatgccgcggtgaatacgttcccgggcct

SEQ ID NO: 83 - DNA, 121 bp, Unknown
  16S DNA Tag
ggggagtgccttcgggaaccagaacacaggtgctgcatggctgtcgtcagctcgtgtcgtgagatgttgggttaagtcccgcaacgagcgcaacccttgtccttagttgccagcacgtaaa

SEQ ID NO: 84 - DNA, 250 bp, Unknown
  16S DNA Tag
cacgccctaaacgatgcgaactggatgttggtctcaactcggagatcagtgtcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtccggaatcctgcagagatgcgggagtgccttcgggaatcggaacacaggtgctgc

SEQ ID NO: 85 - DNA, 588 bp, Unknown
  16S DNA Tag
cacgccctaaacgatgcgaactggatgttggtctcaactcggagatcagtgtcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtccggaatcctgcagagatgcgggagtgccttcgggaatcggaacacaggtgctgcatggctgtcgtcagctcgtgtcgtgagatgttgggttaagtcccgcaacgagcgcaacccttgtccttagttgccagcgagtaatgtcgggaactctaaggagactgccggtgacaaaccggaggaaggtggggatgacgtcaagtcatcatggcccttacggccagggctacacacgtactacaatggtcggtacagagggttgcaataccgcgaggtggagccaatcccagaaagccgatcccagtccggatcggagtctgcaactcgactccgtgaagtcggaatcgctagtaatcgcggatcagctatgccgcggtgaatacgttcccgggcct

SEQ ID NO: 86 - DNA, 247 bp, Unknown
  16S DNA Tag
catgccgtaaacgttgggcgctaggtgtggggaccttccacggtctccgtgccgcagctaacgcattaagcgccccgcctggggagtacggccgcaaggctaaaactcaaaggaattgacgggggcccgcacaagcggcggagcatgctgattaattcgatgcaacgcgaagaaccttacctgggtttgacatataccggaaagctgcagagatgtagcccccttttggtcggtatacaggtggtgc

SEQ ID NO: 87 - DNA, 585 bp, Unknown
  16S DNA Tag
catgccgtaaacgttgggcgctaggtgtggggaccttccacggtctccgtgccgcagctaacgcattaagcgccccgcctggggagtacggccgcaaggctaaaactcaaaggaattgacgggggcccgcacaagcggcggagcatgctgattaattcgatgcaacgcgaagaaccttacctgggtttgacatataccggaaagctgcagagatgtagcccccttttggtcggtatacaggtggtgcatggctgtcgtcagctcgtgtcgtgagatgttgggttaagtcccgcaacgagcgcaaccctcgtcctatgttgccagcacgtaatggtggggactcataggagactgccggggtcaactcggaggaaggtggggatgacgtcaagtcttcatgccccttatgtccagggcttcaagcatgctacaatggccggtacaaagggctgcgaaaccgcaaggtggagcgaatcccaaaaagccggtctcagttcggattggggtctgcaactcgaccccatgaagtcggagtcgctagtaatcgcagatcagcaacgctgcggtgaatacgttcccgggcct

SEQ ID NO: 88 - DNA, 594 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacgccggaaagcatcagagatggtgccccccttgtggtcggtgtacaggtggtgcatggctgtcgtcagctcgtgtcgtgagatgttgggttaagtcccgcaacgagcgcaacccttgtcctgtgttgccagcatgcccttcggggtgatggggactcacaggagaccgccggggtcaactcggaggaaggtggggacgacgtcaagtcatcatgccccttatgtcttgggctgcacacgtgctacaatggcaggtacaatgagctgcgataccgtgaggtggagcgaatctcaaaaagcctgtctcagttcggattggggtctgcaactcgaccccatgaagtcggagttgctagtaatcgcagatcagcattgctgcggtgaatacgttcccgggcct

SEQ ID NO: 89 - DNA, 252 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 90 - DNA, 587 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcaaagccgcgaggcctagccaatcccataaaaccattctcagttcggattgtaggctgcaactcgcctacatgaagccggaatcgctagtaatcgcggatcagcatgccgcggtgaatacgttcccgggcct

SEQ ID NO: 91 - DNA, 586 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggcct

SEQ ID NO: 92 - DNA, 588 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgaaccggaaagacctggaaacaggtgccccgcttgcggtcggtttacaggtggtgcatggttgtcgtcagctcgtgtcgtgagatgttgggttaagtcccgcaacgagcgcaaccctcgttctatgttgccagcacgtgatggtggggactcataggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggcct

SEQ ID NO: 93 - DNA, 248 bp, Unknown
  16S DNA Tag
cacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgc

SEQ ID NO: 94 - DNA, 583 bp, Unknown
  16S DNA Tag
cacgccgtaaacgatgaatgccagctgttggggtgcttgcaccgcagtagcgcagctaacgctttgagcattccgcctggggagtacggtcgcaagattaaaactcaaaggaattgacgggggcccgcacaagcggtggagcatgtggtttaattcgaagcaacgcgcagaaccttaccatcctttgacatggcgtgttacccagagagatttggggtccacttcggtggcgcgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaggagcgatctggagcaaatccccaaaagccgtctcagttcggattgcactctgcaactcgagtgcatgaaggcggaatcgctagtaatcgtggatcagcatgccacggtgaatacgttcccgggcct

SEQ ID NO: 95 - DNA, 250 bp, Unknown
  16S DNA Tag
cacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaagtcttgacatccctctgaatcctctagagatagaggcggccttcgggacagaggtgacaggtggtgc

SEQ ID NO: 96 - DNA, 588 bp, Unknown
  16S DNA Tag
cacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaagtcttgacatccctctgaatcctctagagatagaggcggccttcgggacagaggtgacaggtggtgcatggttgtcgtcagctcgtgtcgtgagatgttgggttaagtcccgcaacgagcgcaacccttgattttagttgccagcaggtaaggctgggcactctagaatgactgccggtgacaaaccggaggaaggcggggatgacgtcaaatcatcatgccccttatgacttgggctacacacgtactacaatggctggtacaacgggaagcgaagccgcgaggtggagccaatcctataaaagccagtctcagttcggattgcaggctgcaactcgcctgcatgaagtcggaattgctagtaatcgcggatcagcatgccgcggtgaatacgttcccgggtct

SEQ ID NO: 97 - DNA, 252 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgc

SEQ ID NO: 98 - DNA, 587 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgcaggctgcaactcgcctgcatgaagccggaatcgctagtaatcgcggatcagcatgccgcggtgaatacgttcccgggcct

SEQ ID NO: 99 - DNA, 250 bp, Unknown
  16S DNA Tag
cacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgc

SEQ ID NO: 100 - DNA, 585 bp, Unknown
  16S DNA Tag
cacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccacaaatctgttctcagttcggatcgcagtctgcaactcgactgcgtgaagctggaatcgctagtaatcgcggatcagcatgccgcggtgaatacgttcccgggcct

SEQ ID NO: 101 - DNA, 250 bp, Unknown
  16S DNA Tag
cacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgc

SEQ ID NO: 102 - DNA, 585 bp, Unknown
  16S DNA Tag
cacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggcct

SEQ ID NO: 103 - DNA, 249 bp, Unknown
  16S DNA Tag
cacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgc

SEQ ID NO: 104 - DNA, 586 bp, Unknown
  16S DNA Tag
cacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaatgtcgcggtgaatacgttcccgggcct

SEQ ID NO: 105 - DNA, 249 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgc

SEQ ID NO: 106 - DNA, 249 bp, Unknown
  16S DNA Tag
cacgccgtaaacgatgtcaactagccgttgggttccttgagaacttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgc

SEQ ID NO: 107 - DNA, 586 bp, Unknown
  16S DNA Tag
cacgccgtaaacgatgtcaactagccgttgggttccttgagaacttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgttatggtgggaactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggcct

SEQ ID NO: 108 - DNA, 248 bp, Unknown
  16S DNA Tag
catgccgtaaacgttgggaactaggtgtgggtctcattccacgagatccgtgccgcagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatacaccggaatcactcagagatgggtgcgtcttcggactggtgtacaggtggtgc

SEQ ID NO: 109 - DNA, 586 bp, Unknown
  16S DNA Tag
catgccgtaaacgttgggaactaggtgtgggtctcattccacgagatccgtgccgcagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatacaccggaatcactcagagatgggtgcgtcttcggactggtgtacaggtggtgcatggttgtcgtcagctcgtgtcgtgagatgttgggttaagtcccgcaacgagcgcaaccctcgttctatgttgccagcacgtcatggtggggactcataggagactgccggggtcaactcggaggaaggtggggatgacgtcaaatcatcatgccccttatgtcttgggcttcacgcatgctacaatggccggtacaaagggctgcgaaaccgcgaggtggagcgaatcccaaaaaaccggtctcagttcggattggggtctgcaactcgaccccatgaagtcggagtcgctagtaatcgcagatcagcaacgctgcggtgaatacgttcccgggcct

SEQ ID NO: 110 - DNA, 250 bp, Unknown
  16S DNA Tag
cacgccctaaacgatgtcaactggttgttgggaaggttccttctcagtaacgtagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgtctagaattttgcagagatgtgaaagtgctcgaaagagaactagaacacaggtgctgc

SEQ ID NO: 111 - DNA, 580 bp, Unknown
  16S DNA Tag
cacgccctaaacgatgtcaactggttgttgggaaggttccttctcagtaacgtagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgtctagaattttgcagagatgtgaaagtgctcgaaagagaactagaacacaggtgctgcatggccgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaggtcctcatggcccttatgggtagggctacacacgtcatacaatggccggtacagagggctgccaacccgcgagggggagccaatcccagaaaaccggtcgtagtccggatcgcagtctgcaactcgactgcgtgaagtcggaatcgctagtaatcgcggatcagcttgccgcggtgaatacgttccccgggtc

SEQ ID NO: 112 - DNA, 93 bp, Unknown
  16S DNA Tag
ggtcgtagtccggatcgcagtctgcaactcgactgcgtgaagtcggaatcgctagtaatcgcggatcagcttgccgcggtgaatacgttcccc

SEQ ID NO: 113 - DNA, 250 bp, Unknown
  16S DNA Tag
cacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaaaaaccttaccaagtcttgacatccctctgaatcctctagagatagaggcggccttcgggacagaggtgacaggtggtgc

SEQ ID NO: 114 - DNA, 588 bp, Unknown
  16S DNA Tag
cacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaaaaaccttaccaagtcttgacatccctctgaatcctctagagatagaggcggccttcgggacagaggtgacaggtggtgcatggttgtcgtcagctcgtgtcgtgagatgttgggttaagtcccgcaacgagcgcaacccttgattttagttgccagcaggtaaggctgggcactctagaatgactgccggtgacaaaccggaggaaggcggggatgacgtcaaatcatcatgccccttatgacttgggctacacacgtactacaatggctggtacaacgggaagcgaagccgcgaggtggagccaatcctataaaagccagtctcagttcggattgcaggctgcaactcgcctgcatgaagtcggaattgctagtaatcgcggatcagcatgccgcggtgaatacgttcccgggtct

SEQ ID NO: 115 - DNA, 84 bp, Unknown
  16S DNA Tag
ctgaaactcaaaggaattgacggggacccgcacaagcagtggagtatgtggtttaattcgaagcaacgcgaaaaaccttaccaa

SEQ ID NO: 116 - DNA, 247 bp, Unknown
  16S DNA Tag
cacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcatattgaccgtgggtgaaagctcactttgtagcaatacacaatttgtaaggtgctgc

SEQ ID NO: 117 - DNA, 585 bp, Unknown
  16S DNA Tag
cacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcatattgaccgtgggtgaaagctcactttgtagcaatacacaatttgtaaggtgctgcatggctgtcgtcagctcgtgccgtgaggtgttgggttaagtcccgcaacgagcgcaacccccatcattagttgccatcaggtaaagctgggaactctaatgaaactgccgtcgtaagacgcgaggaaggaggggatgatgtcaagtcatcatggcctttatgcccagggctacacacgtgctacaatggaagggacaaagagctgccacttggcgacaaggcgctaatctcaaaaaccctttctcagttcagatcgcagtctgcaactcgactgcgtgaagctggaatcgctagtaatcgtatatcagcaatgatacggtgaatacgttcccggacct

SEQ ID NO: 118 - DNA, 247 bp, Unknown
  16S DNA Tag
cacaccgtaaacgttgggcgctaggtgtgggatccattccacgggttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatatgccggaaagctctagagatagagccccttttagtcggtatacaggtggtgc

SEQ ID NO: 119 - DNA, 585 bp, Unknown
  16S DNA Tag
cacaccgtaaacgttgggcgctaggtgtgggatccattccacgggttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatatgccggaaagctctagagatagagccccttttagtcggtatacaggtggtgcatggctgtcgtcagctcgtgtcgtgagatgttgggttaagtcccgcaacgagcgcaaccctcgtcctatgttgccagcgggtaatgccggggactcataggagactgccggggtcaactcggaggaaggtggggatgacgtcaagtcatcatgccccttatgtccagggcttcacgcatgctacaatggccggtacaaagggctgcgatgctgtaaggcggagcgaatcccaaaaagccggtctcagttcggattggggtctgcaactcgaccccatgaagtcggagtcgctagtaatcgcagatcagcaacgctgcggtgaatacgttcccgggcct

SEQ ID NO: 120 - DNA, 285 bp, Unknown
  16S DNA Tag
cgggttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatatgccggaaagctctagagatagagccccttttagtcggtatacaggtggtgcatggctgtcgtcagctcgtgtcgtgagatgttgggttaagtcccgcaacgagcgcaaccctcgtcctatgttgccagcg

SEQ ID NO: 121 - DNA, 248 bp, Unknown
  16S DNA Tag
cacgctgtaaacgatgattgctagttgtcagccggcatgccggttggtgacgcagctaacgcattaagcaatccgcctggggagtacggtcgcaagattaaaactcaaaggaattgacgggggcccgcacaagcggtggagcatgtggtttaattcgaagcaacgcgcagaaccttaccaccttttgacatgcctggacatcatgggagaccatgctttctcttcggagactgggacacaggtgctgc

SEQ ID NO: 122 - DNA, 561 bp, Unknown
  16S DNA Tag
cacgctgtaaacgatgattgctagttgtcagccggcatgccggttggtgacgcagctaacgcattaagcaatccgcctggggagtacggtcgcaagattaaaactcaaaggaattgacgggggcccgcacaagcggtggagcatgtggtttaattcgaagcaacgcgcagaaccttaccaccttttgacatgcctggacatcatgggagaccatgctttctcttcggagactgggacacaggtgctgcatggctgtcgtcagctcgtgtcgtgagatgttgggttaagtcccgcaacgagcgcaaccctcgctgttagttgccatcatttagttgggaactctaacaggactgccggtgctaagccggaggaaggtggggatgacgtcaagtcctcatggcccttacagggtgggctacacacgtgctacaatggcgactacagagggcaaatccctaaaagtcgtctcagttcggatcgtcctctgcaactcgagggcgtgaagttggaatcgctagtaatcgcggatcagcatgccgcggtgaatacgttcccgggcct

SEQ ID NO: 123 - DNA, 250 bp, Unknown
  16S DNA Tag
cacgccgtaaacgatggatgctagctgtggggggtatcgaccccttccgtagcgaagctaacgcgttaagcatcccgcctgtggagtacggccgcaaggctaaaacataaaggaattgacggggacccgcacaagtggtggagcgtgttctttaattcgatgataaacgaaaaaccttaccagggtttgacatcccaagaattttgtcgaaagacgagagtgctttattgaacttggtgacagctgttgc

SEQ ID NO: 124 - DNA, 572 bp, Unknown
  16S DNA Tag
cacgccgtaaacgatggatgctagctgtggggggtatcgaccccttccgtagcgaagctaacgcgttaagcatcccgcctgtggagtacggccgcaaggctaaaacataaaggaattgacggggacccgcacaagtggtggagcgtgttctttaattcgatgataaacgaaaaaccttaccagggtttgacatcccaagaattttgtcgaaagacgagagtgctttattgaacttggtgacagctgttgcatggccgtcgtcagctcgtgtcgtgagatgtttggttaagtccatcaacgagcgcaacccttatagttagttggatttttctagctagactgccccggtaacggggaggaaggaggggatgatgtcaggtcagtattacgcttacatcctgggctagaaacgcgctacaatggccggtacaacgggcagccaagccgcaaggcggagcaaatcccaacaaagccggtcccagttcggattggaggctgaaactcgcctccatgaagtcggaatcactagtaatcgcaaatcagcatgttgcggtgaatacgttcccgggtct

SEQ ID NO: 125 - DNA, 92 bp, Unknown
  16S DNA Tag
ttccgtagcgaagctaacgcgttaagcatcccgcctgtggagtacggccgcaaggctaaaacataaaggaattgacggggacccgcacaagt

SEQ ID NO: 126 - DNA, 250 bp, Unknown
  16S DNA Tag
cacgccgtaaacgatggatactagctgttggaggtatcgaccccttcagtagcgaagctaacgcgttaagtatcccgcctgtggagtacggtcgcaagactaaaacataaaggaattgacggggacccgcacaagcggtggagcgtgttctttaattcgatgataaacgaagaaccttaccagggcttgacatcccttgaattttgtcgaaagacgagagtgctttattgagcaaggtgacaggtgttgc

SEQ ID NO: 127 - DNA, 572 bp, Unknown
  16S DNA Tag
cacgccgtaaacgatggatactagctgttggaggtatcgaccccttcagtagcgaagctaacgcgttaagtatcccgcctgtggagtacggtcgcaagactaaaacataaaggaattgacggggacccgcacaagcggtggagcgtgttctttaattcgatgataaacgaagaaccttaccagggcttgacatcccttgaattttgtcgaaagacgagagtgctttattgagcaaggtgacaggtgttgcatggccgtcgtcagctcgtgtcgtgagatgtttggttaagtccatcaacgagcgcaacccttgtgattagttgtatttttctaatcaaactgccccggtaacggggaggaaggaggggatgatgtcaggtcagtattacccttacaccctgggctagaaacgcgctacaatggccagtacaatgggcagcgaagtcgcgagatggagcaaatcgcatcaaagctggtcccagttcggatagtaggctgaaactcgcctgcttgaagtgggaatcgctagtaatcgcaaatcagcatgttgcggtgaatacgttcccgggtct

SEQ ID NO: 128 - DNA, 185 bp, Unknown
  16S DNA Tag
gatggatactagctgttggaggtatcgaccccttcagtagcgaagctaacgcgttaagtatcccgcctgtggagtacggtcgcaagactaaaacataaaggaattgacggggacccgcacaagcggtggagcgtgttctttaattcgatgataaacgaagaaccttaccagggcttgacatccct

SEQ ID NO: 129 - DNA, 594 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaaagagctgcgaaaccgtgaggtggagcgaatctcaaaaagccggtctcagttcggattggggtctgcaactcgaccccatgaagtcggagttgctagtaatcgcagatcagcattgctgcggtgaatacgttcccgggcct

SEQ ID NO: 130 - DNA, 594 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggcaggtacaaagagctgcgaagccgtgaggcggagcgaatctcaaaaagcctgtctcagttcggattggggtctgcaactcgaccccatgaagtcggagttgctagtaatcgcagatcagcattgctgcggtgaatacgttcccgggcct

SEQ ID NO: 131 - DNA, 250 bp, Unknown
  16S DNA Tag
cacgccgtaaacgatggatgctagctgtggggggtatcgaccccttccgtagcgaagctaacgcgttaagcatcccgcctgtggagtacggccgcaaggctaaaacataaaggaattgacggggacccgcacaagtggaggagcgtgttctttaattcgatgataaacgaaaaaccttaccagggtttgacatcccaagaattttgtcgaaagacgagagtgctttattgaacttggtgacagctgttgc

SEQ ID NO: 132 - DNA, 92 bp, Unknown; identical to SEQ ID NO: 125
  16S DNA Tag

SEQ ID NO: 133 - DNA, 1419 bp, Unknown
  16S DNA Tag
atggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtgtggtagagggtggtggaatttcctgtgtagcggtgaaatgcgtagatataggaaggaacaccagtggcgaaggcgaccacctggaccaacactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctag

SEQ ID NO: 134 - DNA, 1827 bp, Unknown
  16S DNA Tag
cggtaaggctccttcgggagtacacgagcggcgaacgggtgagtaacacgtgagcaatctgcccttcacatcgggataactccgggaaaccgaagctaataccgaatacgaccacttccggcatcggatggtggtggaaagttccggcggtgaaggatgagctcgcggcctatcagcttgttggtggggtaacggcccaccaaggcttcgacgggtagccggcctgagagggtgaccggccacactgggactgagacacggcccagactcctacgggaggcagcagtggggaatattggacaatgggcgaaagcctgatccagcaacgccgcgtgagggatgacggccttcgggttgtaaacctctttcagcagggacgaagcgcaagtgacggtacctgcagaagaagcaccggccaactacgtgccagcagccgcggtaatacgtagggtgcgagcgttgtccggaattattgggcgtaaagggctcgtaggcggtctgtcgcgtcgggagtgaaaactcagggcttaaccctgagcctgcttccgatacgggcagactagaggtatgcaggggagaacggaattcctggtgtagcggtgaaatgcgcagatatcaggaggaacaccggtggcgaaggcggttctctgggcattacctgacgctgaggagcgaaagtgtggggagcgaacaggattagataccctggtagtccacaccgtaaacgttgggcgctaggtgtgggactcattccacgagttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctaggtttgacatatagggaaatctgctagagatagcaggtccgtaagggctctatacaggtggtgcatggctgtcgtcagctcgtgtcgtgagatgttgggttaagtcccgcaacgagcgcaaccctcgtcttatgttgccagcacgtcatggtggggactcataagagactgccggggtcaactcggaggaaggtggggatgacgtcaagtcatcatgccccttatgcctagggcttcacgcatgctacaatggccggtacaaagggctgcgaaatcgcaagatggagcgaatcccaaaaagccggtctcagttcggattggggtctgcaactcgaccccatgaagtcggagtcgctagtaatcgcagatcagcaacgctgcggtgaatacgttcccgggccttgtacacaccgcccgtcacgtcacgaaagttggcaacacccgaagccggtggcccaacccttgtggagggagccgtcgaaggtggggcgagcgattgggacgaagtcgtaacaaggtagccgtaccggaaggtgcggctggatcacctcctttctaaggagcatcactggcagcctcgagctgtccaggcttccttgtttcgtggacaaacgttccacggcaaggactgctcactagtggaacgtcgattatttggtctgcgaccggacaacatctcgtcagtacttccgccgtcctcggacggatcacggagtggaacctcgagcgtcgcgaacggaagcaaaccaggcacactgttgggtcctgagggatcgagtcactcgacctctccgggcctccatcccctcgaactgccgaccactggtcggcaagcgaggatccgatggcggcaccgcccgtatcttgagaactacacagtggacgcgagcatctttgtagcaagacaagctactaaagggcacatggtg

SEQ ID NO: 135 - DNA, 312 bp, Unknown
  16S DNA Tag
atcgggataactccgggaaaccgaagctaataccgaatacgaccacttccggcatcggatggtggtggaaagttccggcggtgaaggatgagctcgcggcctatcagcttgttggtggggtaacggcccaccaaggcttcgacgggtagccggcctgagagggtgaccggccacactgggactgagacacggcccagactcctacgggaggcagcagtggggaatattggacaatgggcgaaagcctgatccagcaacgccgcgtgagggatgacggccttcgggttgtaaacctctttcagcagggacgaa

SEQ ID NO: 136 - DNA, 252 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggagaccttcagttcggctggaccggtgacaggtgctgc

SEQ ID NO: 137 - DNA, 587 bp, Unknown
  16S DNA Tag
cacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggagaccttcagttcggctggaccggtgacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacgttcccgggcct

SEQ ID NO: 138 - DNA, 1406 bp, Unknown
  16S DNA Tag
aggggagcggcagacgggtgagtaacgcgtgggaatctacccatctctacggaacaactccgggaaactggagctaataccgtatacgtccttttggagaaagatttatcggagatggatgagcccgcgttggattagctagttggtggggtaatggcctaccaaggcgacgatccatagctggtctgagaggatgatcagccacactgggactgagacacggcccagactcctacgggaggcagcagtggggaatattggacaatgggcgcaagcctgatccagccatgccgcgtgagtgatgaaggccctagggttgtaaagctctttcaacggtgaagataatgacggtaaccgtagaagaagccccggctaacttcgtgccagcagccgcggtaatacgaagggggctagcgttgttcggaattactgggcgtaaagcgcacgtaggcggatacttaagtcaggggtgaaatcccggggctcaaccccggaactgcctttgatactgggtatctcgagtccggaagaggtgagtggaattccgagtgtagaggtgaaattcgtagatattcggaggaacaccagtggcgaaggcggctcactggtccggtactgacgctgaggtgcgaaagcgtggggagcaaacaggattagataccctggtagtccacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggagaccttcagttcggctggaccggtgacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagaccgcgaggtcgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggttttacccgaaggcgctgtgctaaccgcaaggaggcaggcgaccacggtagggtcagcgactggggtgaagtcgtaacaaggtagccgtaggggaacctgcggctggatcacctc

SEQ ID NO: 139 - DNA, 279 bp, Unknown
  16S DNA Tag
ccggtactgacgctgaggtgcgaaagcgtggggagcaaacaggattagataccctggtagtccacgccgtaaacgatggaagctagccgttggcaagtttacttgtcggtggcgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatcccggtcgcggtttccagagatggag

SEQ ID NO: 140 - DNA, 634 bp, Unknown
  16S DNA Tag
atggctcagattgaacgctggcggcaggcctaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtg

SEQ ID NO: 141 - DNA, 586 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacattaaggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggcct

SEQ ID NO: 142 - DNA, 117 bp, Unknown
  16S DNA Tag
ggtgccttcgggagcattgagacaggtgctgcatggctgtcgtcagctcgtgtcgtgagatgttgggttaagtcccgtaacgagcgcaacccttgtccttagttaccagcacattaa

SEQ ID NO: 143 - DNA, 585 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgtgatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgtcccgggcct

SEQ ID NO: 144 - DNA, 439 bp, Unknown; identical to SEQ ID NO: 79
  16S DNA Tag

SEQ ID NO: 145 - DNA, 588 bp, Unknown
  16S DNA Tag
cacgccctaaacgatgcgaactggatgttggtctcaactcggagatcagtgtcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtccggaatcctgcagagatgcgggagtgccttcgggaatcggaacacaggtgctgcatggctgtcgtcagctcgtgtcgtgagatgttgggttaagtcccgcaacgagcgcaacccttgtccttagttgccagcacgtaatggtgggaactctaaggagactgccggtgacaaaccggaggaaggtggggatgacgtcaagtcatcatggcccttacggccagggctacacacgtactacaatggtcggtacagagggttgcaataccgcgaggtggagccaatcccagaaagccgatcccagtccggatcgaagtctgcaactcgacttcgtgaagtcggaatcgctagtaatcgcggatcagctatgccgcggtgaatacgttcccgggcct

SEQ ID NO: 146 - DNA, 337 bp, Unknown
  16S DNA Tag
gggagtgccttcgggaatcggaacacaggtgctgcatggctgtcgtcagctcgtgtcgtgagatgttgggttaagtcccgcaacgagcgcaacccttgtccttagttgccagcacgtaatggtgggaactctaaggagactgccggtgacaaaccggaggaaggtggggatgacgtcaagtcatcatggcccttacggccagggctacacacgtactacaatggtcggtacagagggttgcaataccgcgaggtggagccaatcccagaaagccgatcccagtccggatcgaagtctgcaactcgacttcgtgaagtcggaatcgctagtaatcgcg

SEQ ID NO: 147 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacatccagagatgggtgcccccttgtggtcggtgtacaggtggtgc

SEQ ID NO: 148 - DNA, 594 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacatccagagatgggtgcccccttgtggtcggtgtacaggtggtgcatggctgtcgtcagctcgtgtcgtgagatgttgggttaagtcccgcaacgagcgcaacccttgtcccgtgttgccagcatgcccttcggggtgatggggactcacgggagaccgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagttgctagtaatcgcagatcagcagtgctgcggtgaatacgttcccgggcct

SEQ ID NO: 149 - DNA, 248 bp, Unknown
  16S DNA Tag
catgccgtaaacgttgggaactagatgtggggaccattccacggtctccgtgtcgcagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatatagaggaaaagtgcagaaatgtactccccgcaaggtctctatacaggtggtgc

SEQ ID NO: 150 - DNA, 586 bp, Unknown
  16S DNA Tag
catgccgtaaacgttgggaactagatgtggggaccattccacggtctccgtgtcgcagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatatagaggaaaagtgcagaaatgtactccccgcaaggtctctatacaggtggtgcatggttgtcgtcagctcgtgtcgtgagatgttgggttaagtcccgcaacgagcgcaaccctcgtcctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtaaggtggagcgaatcccaaaaagccggtctcagttcggattgaggtctgcaactcgacctcatgaagtcggagtcgctagtaatcgcagatcagcaacgctgcggtgaatacgttcccgggcct

SEQ ID NO: 151 - DNA, 161 bp, Unknown
  16S DNA Tag
tctccgtgtcgcagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatatagaggaaaa

SEQ ID NO: 152 - DNA, 250 bp, Unknown
  16S DNA Tag
cacgccctaaacgatgcgaactggatgttgggtgcaactaggcactcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatccacggaactttccagagatggattggtgccttcgggaaccgtgagacaggtgctgc

SEQ ID NO: 153 - DNA, 588 bp, Unknown
  16S DNA Tag
cacgccctaaacgatgcgaactggatgttgggtgcaactaggcactcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatccacggaactttccagagatggattggtgccttcgggaaccgtgagacaggtgctgcatggctgtcgtcagctcgtgtcgtgagatgttgggttaagtcccgcaacgagcgcaacccttgtccctagttgccagcacgtaatggtgggaactctagggagaccgccggtgacaaaccggaggaaggtggggatgacgtcaagtcatcatggcccttacggccagggctacacacgtactacaatggaaaggacagagggctgcgatcccgcgagggtgagccaatcccagaaaccttttctcagtccggattggagtctgcaactcgactccatgaagtcggaatcgctagtaatcgcagatcagcattgctgcggtgaatacgttcccgggcct

SEQ ID NO: 154 - DNA, 250 bp, Unknown
  16S DNA Tag
cacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtcaggaacctccgagagatcggagggtgcccgaaagggagcctgaacacaggtgctgc

SEQ ID NO: 155 - DNA, 580 bp, Unknown
  16S DNA Tag
cacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtcaggaacctccgagagatcggagggtgcccgaaagggagcctgaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggatcgcagtctgcaactcgactgcgtgaagttggaatcgctagtaatcgcggatcagcatgccgcggtgaatacgttcccgggtct

SEQ ID NO: 156 - DNA, 520 bp, Unknown
  16S DNA Tag
acgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtcaggaacctccgagagatcggagggtgcccgaaagggagcctgaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggatcgcagtctgcaactcgactgcgtg

SEQ ID NO: 157 - DNA, 248 bp, Unknown
  16S DNA Tag
cacgccgtaaacgttgggaactaggtgtgggccacattccacgtggtctgtgccgcagctaacgcattaagttccccgcctggggagtacggccgcaaggctaaaactcaaaggaattgacgggggcccgcacaagcggcggagcatgtggcttaattcgatgcaacgcgaagaaccttacctgggtttgacatgcagggaaatctcgtagagatacggggtccgtaagggccttgcacaggtggtgc

SEQ ID NO: 158 - DNA, 586 bp, Unknown
  16S DNA Tag
cacgccgtaaacgttgggaactaggtgtgggccacattccacgtggtctgtgccgcagctaacgcattaagttccccgcctggggagtacggccgcaaggctaaaactcaaaggaattgacgggggcccgcacaagcggcggagcatgtggcttaattcgatgcaacgcgaagaaccttacctgggtttgacatgcagggaaatctcgtagagatacggggtccgtaagggccttgcacaggtggtgcatggctgtcgtcagctcgtgtcgtgagatgttgggttaagtcccgcaacgagcgcaaccctcgtcctatgttgccagcgagtaatgtcggggactcataggagactgccggggtcaactcggaggaaggtggggatgacgtcaagtcatcatgccccttatatccagggctgcacacatgctacaatggccggtacaaagagctgcgataccgcaaggtggagcgaatctcataaagccggtctcagttcggattggggtctgcaactcgaccccatgaagtcggagtcgctagtaatcgcagatcagcaacgctgcggtgaatacgttcccgggcct

SEQ ID NO: 159 - DNA, 196 bp, Unknown
  16S DNA Tag
tccccgcctggggagtacggccgcaaggctaaaactcaaaggaattgacgggggcccgcacaagcggcggagcatgtggcttaattcgatgcaacgcgaagaaccttacctgggtttgacatgcagggaaatctcgtagagatacggggtccgtaagggccttgcacaggtggtgcatggctgtcgtcagctcgtg

SEQ ID NO: 160 - DNA, 250 bp, Unknown
  16S DNA Tag
cacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtcaggaacctccgagagattggagggtgcccgaaagggagcctgaacacaggtgctgc

SEQ ID NO: 161 - DNA, 580 bp, Unknown
  16S DNA Tag
cacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtcaggaacctccgagagattggagggtgcccgaaagggagcctgaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggatcgcagtctgcaactcgactgcgtgaagttggaatcgctagtaatcgcggatcagcatgccgcggtgaatacgttcccgggtct

SEQ ID NO: 162 - DNA, 252 bp, Unknown
  16S DNA Tag
cacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgc

SEQ ID NO: 163 - DNA, 587 bp, Unknown
  16S DNA Tag
cacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggcct

SEQ ID NO: 164 - DNA, 586 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggcct

SEQ ID NO: 165 - DNA, 250 bp, Marmoricola scoriae; identical to SEQ ID NO: 4

SEQ ID NO: 166 - DNA, 252 bp, Mesorhizobium opportunistum; identical to SEQ ID NO: 58

SEQ ID NO: 167 - DNA, 249 bp, Streptomyces graminilatus; identical to SEQ ID NO: 38

SEQ ID NO: 168 - DNA, 252 bp, Mesorhizobium shangrilense; identical to SEQ ID NO: 43

SEQ ID NO: 169 - DNA, 250 bp, Dyella ginsengisoli; identical to SEQ ID NO: 84

SEQ ID NO: 170 - DNA, 249 bp, Pseudomonas putida; identical to SEQ ID NO: 103

SEQ ID NO: 171 - DNA, 250 bp, Burkholderia caledonica; identical to SEQ ID NO: 75

SEQ ID NO: 172 - DNA, 249 bp, Streptomyces canus; identical to SEQ ID NO: 24

SEQ ID NO: 173 - DNA, 249 bp, Streptomyces coelicoflavus
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaagcatcagagatggtgccccccttgtggtcggtgtacaggtggtgc

SEQ ID NO: 174 - DNA, 252 bp, Arthrobacter globiformis
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaccgccgcagagatgtggtttctccttttggggccggttcacaggtggtgc

SEQ ID NO: 175 - DNA, 249 bp, Pseudomonas brassicacearum; identical to SEQ ID NO: 14

SEQ ID NO: 176 - DNA, 250 bp, Unknown
  16S DNA Tag
cacgccctaaacgatgcgaactgggtgttgggtgcaacttggcactcagtatcgaagctaacgcgttaagttcgccgcctgggaagtacggtcgcaagactgaaactcaaaggaattgacgggggcccgcacaagcggtggagtatgtggtttaattcgatgcaacgcgaagaaccttacctggccttgacatgcacggaaccctgcagagatgcgggggtgccttcgggaaccgtgacacaggtgctgc

SEQ ID NO: 177 - DNA, 255 bp, Unknown
  16S DNA Tag
ctagccttaaacgatgaatgcttggtgtctggggttataaagtccccgggtgccgtcgctaacgctttaagcattccgcctggggagtacggtcgcaagactgaaactcaaaggaattgacggggacccgcacaagcggtggagcatgtggtttaattcgacgcaacgcgaagaaccttacctgggctagaatgcctctgacaaccctagagatagggccttctgggtaaaaccagacagagtgcaaggtgctgc

SEQ ID NO: 178 - DNA, 248 bp, Unknown
  16S DNA Tag
ctggccgtaaacgatgccaactgggcgttggggcgaacagcctcagcgccggagcgaacgcggtaagttggccgcctggggagtacgaccgcaaggttaaaactcaaaggaattgacgggggcccgcacaagcagcggagcgtgtggtttaattcgaggcaacgcgaagaaccttacctgggcttgacatgggcatggtagtgaagcgaaagcggaacgacccgcaagggagtgtccacaggtgctgc

SEQ ID NO: 179 - DNA, 250 bp, Unknown
  16S DNA Tag
cacgccgtaaacgatgaatgctaggtgtcaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatcccgctgacgggtctagagataggccgttccttcgggacagcggagacaggtggtgc

SEQ ID NO: 180 - DNA, 249 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaatctgctagagatagcggagtgccttcgggaacattgagacaggtgctgc

SEQ ID NO: 181 - DNA, 249 bp, Unknown
  16S DNA Tag
cacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaatctgctagagatagcggagtgccttcgggagcattgagacaggtgctgc

SEQ ID NO: 182 - DNA, 244 bp, Unknown
  16S DNA Tag
cacactgtaaacgatgatcactcgatgttggcgatacacagccagcgtcttagcaaaagcgataagtgatccacctggggagtacgccggcaacggtgaaactcaaaggaattgacgggggtccgcacaagcggtggagcatgtggtttaattcgatgatacgcgaggaaccttacctgggctagaatgcccttgacgggtccagagacggatcgttccgcaaggacaaggagcaaggtgctgc

SEQ ID NO: 183 - DNA, 249 bp, Unknown
  16S DNA Tag
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcagcggagcatgtggcttaattcgacgcaacgcgaagaaccttaccaaggcttgacatacgccggaaaaccctggagacagggtcccccttgtggtcggtgtacaggtggtgc

SEQ ID NO: 184 - DNA, 250 bp, Unknown
  16S DNA Tag
cacgccgtaaacgatgaatgctaggtgttaggggtttcaatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtacagagatgtacctttccttcgggacagaggagacaggtggtgc

SEQ ID NO: 185 - DNA, 248 bp, Unknown
  16S DNA Tag
cacgccgtaaacggtgggcgctaggtgtgggttccttccacggaatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgc

SEQ ID NO: 186 - DNA, 244 bp, Unknown
  16S DNA Tag
cacactgtaaacgttgatgactcgatgttggcgatacacagccagcgtccaagagcaatcgttaagtcatccacctggggagtacgccggcaacggtgaaactcaaaggaattgacgggggtccgcacaagcggtggagcatgtggtttaattcgatgatacgcgaggaaccttacctggactagaatgcccttgactggtacagagatgtatcgtttcgcaagaacaaggagcaaggtgctgc

SEQ ID NO: 187 - DNA, 244 bp, Unknown
  16S DNA Tag
cacgccctaaacgatgaatactcgctgttagcgatacacagttagcggctaagcgaaagcgttaagtattccacctggggagtacgcccgcaagggtgaaactcaaaggaattgacgggggcccgcacaagcggaggagcatgtggtttaattcgatgatacgcgaggaaccttacccgggcttgaaagttagtgaatgatttagagatagatcagtccgcaaggacacgaaactaggtgctgc

SEQ ID NO: 188 - DNA, 244 bp, Unknown
  16S DNA Tag
cacactgtaaacgttgattactcgctgtgtgcgatatacagtacgcggctaagcgaaagcgataagtaatccacctggggagtacgccggcaacggtgaaactcaaaggaattgacgggggtccgcacaagcggtggagcatgtggtttaattcgatgatacgcgaggaaccttacctgggctagaatgcccttgattgtcccagagatgggaagttccgcaaggacaaggagcaaggtgctgc

SEQ ID NO: 189 - DNA, 244 bp, Unknown
  16S DNA Tag
cacactgtaaacgttgattactcgctgtgtgcgatatacagtacgcggccaagcgaaagcgctaagtaatccacctggggagtacgccggcaacggtgaaactcaaaggaattgacgggggtccgcacaagcggtggagcatgtggtttaattcgatgatacgcgaggaaccttacctgggctagaatgcccttgatgggtccggagacggatcgttccgcaaggacaaggagcaaggtgctgc

SEQ ID NO: 190 - DNA, 251 bp, Unknown
  16S DNA Tag
cacgccctaaacgatgtgggctagacgtcggggggcttgcccctcggtgtcgcagcgaacgcgttaagcccaccgcctggggagtacggccgcaaggttaaaactcaaaggaattgacgggggcccgcacaagcggtggagcatgtggtttaattcgacgcaacgcgcagaaccttaccagcccttgacatggcggttgcgggcttccgagagggggtccttcagttcggctgggccgtcacaggtgctgc

SEQ ID NO: 191 - DNA, 252 bp, Unknown
  16S DNA Tag
cacgctgtaaacgatggatgctagccgttggcgagcttgctcgtcagtggcgcagctaacgctttaagcatcccgcctggggagtacggtcgcaagattaaaactcaaaggaattgacgggggcccgcacaagcggtggagcatgtggttcaattcgaagcaacgcgcagaaccttaccagcccttgacatgtcctctatggattccagagacggagtccttcacttcgggtgggaggaacacaggtgctgc

SEQ ID NO: 192 - DNA, 247 bp, Unknown
  16S DNA Tag
ccagctgtaaacgatgcagactcggtgatgggttggcttcgtgccaacccagtgccgcagggaagccgttaagtctgccgcctggggagtacggtcgcaagactgaaacttaaaggaattggcgggggagcaccacaaggggtgaagcctgcggttcaattggagtcaacgccgggaatcttaccgggggcgacagcagagtgaaggtcaagctgaagactttactagacaagctgagaggaggtgc

SEQ ID NO: 193 - DNA, 252 bp, Unknown
  16S DNA Tag
cacgccgtaaacgatggatgctagccgttggcaggcttgcctgtcagtggcgcagctaacgcattaagcatcccgcctggggagtacggtcgcaagattaaaactcaaaggaattgacgggggcccgcacaagcggtggagcatgtggttcaattcgaagcaacgcgcagaaccttaccaacctttgacatcccgatcgcgggcaccagagatggagcctttcagttcggctggatcggagacaggtgctgc

SEQ ID NO: 194 - DNA, 251 bp, Unknown
  16S DNA Tag
ctagccctaaacgatggatacttggtgtgactgggattgaatccagtcgtgccgaagctaacgcattaagtatcccgcctggggagtacggtcgcaaggctgaaactcaaaggaattgacgggggcccgcacaagcggtggagtatgtggtttaattcgacgcaacgcgaagaaccttacctgggctcgaacggctagtgacaggtggcgaaagtcgcttttcccgcaagggacactagtcgaggtgctgc

SEQ ID NO: 195 - DNA, 252 bp, Unknown
  16S DNA Tag
cacgccgtaaacgatggatgctagccgttggcgagcttgctcgtcagtggcgcagctaacgctttaagcatcccgcctggggagtacggtcgcaagattaaaactcaaaggaattgacgggggcccgcacaagcggtggagcatgtggttcaattcgaagcaacgcgcagaaccttaccagcccttgacatgtcctctatggattccagagacggagtccttcacttcgggtgggaggaacacaggtgctgc

SEQ ID NO: 196 - DNA, 252 bp, Unknown
  16S DNA Tag
cacgccgtaaacgatgagtgctaagtgttagagggtttccgccctttagtgctgcagtaaacgcattaagcactccgcctggggagtacggccgcaaggctgaaactcaaaggaattgacggggacccgcacaagcggtggagcatgtggtttaattcgaagcaacgcgaagaaccttaccaggtcttgacatcctctgacaatcctagagataggactttccccttcgggggacagagtgacaggtggtgc

SEQ ID NO: 197 - DNA, 252 bp, Unknown
  16S DNA Tag
cacgccgtaaacgatgagtgctaagtgttagagggtttccgccctttagtgctgcagcaaacgcattaagcactccgcctggggagtacggccgcaaggctgaaactcaaaggaattgacggggacccgcacaagcggtggagcatgtggtttaattcgaagcaacgcgaagaaccttaccaggtcttgacatcctctgaccatcctagagataggaccttccccttcgggggacagagtgacaggtggtgc

SEQ ID NO: 198 - DNA, 248 bp, Unknown
  16S DNA Tag
ctggccctaaacgatgagtgcttggtgtggcgggtatcgatccctgccgtgccgaagctaacgcattaagcactccgcctggggagtacggtcgcaaggctgaaactcaaaggaattgacgggggcccgcacaagcggtggagcatgtggttcaattcgacgcaacgcgaagaaccttacctgggctcgaaatgcagatgacattcggcgaaagccggctcccgcaagggcatctgtataggtgctgc

SEQ ID NO: 199 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgaccactcctgaaaaggagcttccgcaaggacactcgtagaggtgctgc

SEQ ID NO: 200 - DNA, 249 bp, Unknown
  16S DNA Tag
cacgccgtaaacgatgagtgctaggtgtggggggtgtcgactccctccgtgccgcagctaacgcattaagcactccgcctggggagtacggccgcaaggctaaaactcaaaggaattgacgggggcccgcacaagcagcggagcatgtggtttaattcgacgcgacgcgaagaaccttacctaggcttgacatgcacgggaattgtctagaaataggcaagcccttcggggctcgtgcacaggtggtgc

SEQ ID NO: 201 - DNA, 252 bp, Unknown
  16S DNA Tag
cacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggcgttccccttcgggggacagagtgacaggtggtgc

SEQ ID NO: 202 - DNA, 248 bp, Unknown
  16S DNA Tag
ctggccctaaacgatgaatgcttggtgtgacgggtatcgatccctgtcgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggctcgaacggcgactgacatcctgcgaaagcaggctcccgcaagggcagtcgtcgaggtgctgc

SEQ ID NO: 203 - DNA, 257 bp, Unknown
  16S DNA Tag
cgcgccgtaaacgatggtcactaggtgtgcgaggtatcgacccctcgcgtgccgccgctaacgcagtaagtgacccgcctggggagtacggccgcaaggttgaaactcaaaggaattgacgggggcccgcacaagcggtggagcatggggattaattcgtcactaaccgaagaaccttacccaggcttgacatccgctgtaagtccgtgaaagcggtaccccttgctcgaaagagtgagcagcgagacaggtgttgc

SEQ ID NO: 204 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaactgcgagtgaccacgtccgaaagggcgcttccgcaaggacactcgtagaggtgctgc

SEQ ID NO: 205 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaactgcgagtgacatcctacgaaagtaggcttccgcaaggacactcgtagaggtgctgc

SEQ ID NO: 206 - DNA, 249 bp, Unknown
  16S DNA Tag
cacgctgtaaacgatgggcactaggtgtgggaggtgtcgactcctcccgtgccgtcgctaacgcattaagtgccccgcctggggagtacggccgcaaggctaaaactcaaaggaattgacgggggcccgcacaagcagcggagcatgtggtttaattcgacgcaacgcgaagaaccttacctgggcttgacatgtaggtgaccggtgtggaaacacacctttccttcgggacaccttcacaggtggtgc

SEQ ID NO: 207 - DNA, 249 bp, Unknown
  16S DNA Tag
cacgccgtaaacgatgggtgctaggtgtgggcggtgtcgactccgtccgtgccgaagctaacgcattaagcaccccgcctggggagtacggccgcaaggctaaaactcaaaggaattgacggggacccgcacaagcagcggagcatgttgtttaattcgacgcgacgcgaagaaccttaccaaggcttgacatgcacgggaatgtcgtagaaatacggcagcccttcggggctcgtgcacaggtggtgc

SEQ ID NO: 208 - DNA, 248 bp, Unknown
  16S DNA Tag
ctagccctaaacgatgaatgtttggtgtggcgggtatcgatccctgccgtgccgaagctaacgcattaaacattccgcctggggagtacggtcgcaaggctgaaactcaaaggaattgacgggggcccgcacaagcggtggagcatgtggttcaattcgacgcaacgcgaagaaccttacccaggctcgaacggcaagtgacatccggtgaaagccggctcccgcaagggcagttgccgaggtgctgc

SEQ ID NO: 209 - DNA, 249 bp, Unknown
  16S DNA Tag
cacgccgtaaacgatgggtgctaggtgtgggcggtgtcgactccgtccgtgccgaagctaacgcattaagcaccccgcctggggagtacggccgcaaggctaaaactcaaaggaattgacgggggcccgcacaagcagcggagcatgtggtttaattcgacgcgacgcgaagaaccttaccagggcttgacatgcactggaatgtcgtagaaatacggcagcccttcggggctagtgcacaggtggtgc

SEQ ID NO: 210 - DNA, 252 bp, Unknown
  16S DNA Tag
cacgccgtaaacgatggatgctagccgttggcgggtttacccgtcagtggcgcagctaacgcattaagcatcccgcctggggagtacggtcgcaagattaaaactcaaaggaattgacgggggcccgcacaagcggtggagcatgtggttcaattcgaagcaacgcgcagaaccttaccagcccttgacatgtcccgtatgggtttcagagatgagacccttcagttcggctggcgggaacacaggtgctgc

SEQ ID NO: 211 - DNA, 247 bp, Unknown
  16S DNA Tag
ccagctgtaaacgatgcagactcggtgatgggttggcctcgcgccaacccagtgccgcagggaagccgttaagtctgccgcctggggagtacggtcgcaagactgaaacttaaaggaattggcgggggagcaccacaaggggtgaagcctgcggttcaattggagtcaacgccgggaatcttaccgggggcgacagcagtatgaaggtcaagccgaagactttaccagacaagctgagaggaggtgc

SEQ ID NO: 212 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgacaaactccgaaaggagtcttccgcaaggacactcgtagaggtgctgc

SEQ ID NO: 213 - DNA, 247 bp, Unknown
  16S DNA Tag
ccagctgtaaacgatgcagactcggtgatgagttggctatatgccaactcagtgccgcagggaagccgttaagtttgccgcctggggagtacggtcgcaagactgaaacttaaaggaattggcgggggagcaccacaaggggtgaagcctgcggttcaattggagtcaacgccggaaatcttaccgggggcgacagcagtatgaaggtcaagctgaagactttaccagacaagctgagaggaggtgc

SEQ ID NO: 214 - DNA, 248 bp, Unknown
  16S DNA Tag
cacgcagtaaacgatgcagactaggcgtggggggacttgaccccctccgtgccggagccaacgcgggaagtctgccgcctggggagtacggccgcaaggctaaaactcaaaggaattgacgggggcccgcacaagcggcggagcgtgcggtttaattcgacgcaacgcgcagaaccttaccagggcttgacatgcttctgcagagccgggaaaccggttggcctttgagggtgaagcacaggtgctgc

SEQ ID NO: 215 - DNA, 247 bp, Unknown
  16S DNA Tag
ccagctgtaaacgatgcagactcggtgatgggatggcctcgagctatcccagtgccgcagggaagccgttaagtctgccgcctggggagtacggtcgcaagactgaaacttaaaggaattggcgggggagcaccacaaggggtgaagcctgcggttcaattggagtcaacgccgggaatcttaccgggggagacagcagagtgaaggtcaagctgaagactttaccagacaagctgagaggaggtgc

SEQ ID NO: 216 - DNA, 247 bp, Unknown
  16S DNA Tag
cacgccgtaaacgttgggcgctaggtgtgggatccttccacggattccgtgccgcagctaacgcattaagcgccccgcctggggagtacggccgcaaggctaaaactcaaaggaattgacgggggcccgcacaagcggcggagcatgttgcttaattcgacgcaacgcgaagaaccttaccaaggtttgacatgcagggaaaagccgtagagatacggtgtccgtaagggtcctgcacaggtggtgc

SEQ ID NO: 217 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgaccagctccgaaaggggccttccgcaaggacactcgtagaggtgctgc

SEQ ID NO: 218 - DNA, 249 bp, Unknown
  16S DNA Tag
cacgccgtaaacgatgggtgctaggtgtgggaggtgtcgactccttccgtgccgtagctaacgcattaagcaccccgcctggggagtacggccgcaaggctaaaactcaaaggaattgacggggccccgcacaagcagcggagcatgtggtttaattcgatgcaacgcgaagaaccttaccagggcttgacatgcgctggaaatccgtagaaatacggccctccttcgggaccagtgcacaggtggtgc

SEQ ID NO: 219 - DNA, 249 bp, Unknown
  16S DNA Tag
cacgctgtaaacgatgggcactaggtgtggggggtgtcgactccctccgtgccgaagctaacgcattaagtgccccgcctggggagtacggccgcaaggctaaaactcaaaggaattgacggggacccgcacaagcagcggagcatgtggtttaattcgacgcaacgcgaagaaccttacctgggcttgacatgtgcctgaccgccctggaaacagggcttcccttcggggcaggttcacaggtggtgc

SEQ ID NO: 220 - DNA, 249 bp, Unknown
  16S DNA Tag
ctagccctaaactatggatgcttggtgtagtgggtacccaatcccactgtgccgcagctaacgcgttaagcatcccgcctggggagtacggtcgcaaggctgaaactcaaaggaattgacgggggcccgcacaagcggtggagcatgtggttcaattcgacgcaacgcgaagaaccttacctgggctcgaagcgcagtggaatgaggtggaaacatctcagcccgcaagggccgctgcggaggtgctgc

SEQ ID NO: 221 - DNA, 249 bp, Unknown
  16S DNA Tag
cacgccgtaaacgatgggcactaggtgtgggaggtgtcgactcctcctgtgccgaagctaacgcattaagtgccccgcctggggagtacggccgcaaggctaaaactcaaaggaattgacggggacccgcacaagcagcggagcatgtggttcaattcgacgcaacgcgaagaaccttacctgggcttgacatgtaggtgacagccgtggaaacacggtctcccttcggggcaccttcacaggtggtgc

SEQ ID NO: 222 - DNA, 249 bp, Unknown
  16S DNA Tag
cacgccgtaaacgatgagtgctaggtgtggggggtgtcgactccctccgtgccgcagctaacgcattaagcactccgcctggggagtacggccgcaaggctaaaactcaaaggaattgacgggggcccgcacaagcagcggagcatgtggtttaattcgacgcgacgcgaagaaccttacctaggcttgacatgcacgggaatgtcgtagaaatacggcagcccttcggggctcgtgcacaggtggtgc

SEQ ID NO: 223 - DNA, 250 bp, Unknown
  16S DNA Tag
cacgccctaaaccatgtcaactgggtgttcgggaagcgatttctgagtaccgtagctaacgcgtgaagttgaccgcctggggagtacggccgcaaggttaaaactcaaaggaattgacggggacccgcacaagcggtggatgatgtggattaattcgatgcaacgcgaacaaccttacctacccttgacatgtccgagagcctgctgagaggcgggtgtgcccgaaagggaatcggaacacaggtgctgc

SEQ ID NO: 224 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggctcgaacggtgatggacatccggcgaaagccggctcccgcaagggccgtcatcgaggtgctgc

SEQ ID NO: 225 - DNA, 245 bp, Unknown
  16S DNA Tag
cacgctgtaaactatgggtgctagccgtcggtcggcatgccggtcggtggcgcagcaaacgcattaagcaccccgcctggggagtacggtcgcaagattaaaactcaaaggaattgacgggggcccgcacaagcggtggagcatgtggtttaattcgaggcaacgcgaagaaccttaccagcccttgacttcccgatcggggctccggagacggagctcttcaattggtcggtgacaggtgctgc

SEQ ID NO: 226 - DNA, 249 bp, Unknown
  16S DNA Tag
cacgccgtaaacgatgggtgctaggtgtggggggtgtcgactccctccgtgccgaagctaacgcattaagcaccccgcctggggagtacggccgcaaggctaaaactcaaaggaattgacgggggcccgcacaagcagcggagcatgttgtttaattcgacgcgacgcgaagaaccttaccaaggcttgacatgcacgggaatgtcgtggaaacacggcagcccttcggggctcgtgcacaggtggtgc

SEQ ID NO: 227 - DNA, 247 bp, Unknown
  16S DNA Tag
ctagccgtaaacgttgggtgctaggtgtgggcggatatcaaccccgtccgtgccgaagctaacgcattaagcaccccgcctggggagtacggccgcaaggctaaaactcaaaggaattgacgggggcccgcacaagcggcggagcatgcggcttaattcgatgcaacgcgaagaaccttacctgggcttgacatgcagggtgaaacccgtgaaagcgggcgctcttcggagcctgcacaggtggtgc

SEQ ID NO: 228 - DNA, 248 bp, Unknown
  16S DNA Tag
ctagccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgacatccggcgaaagccggctcccgcaagggcactcgtagaggtgctgc

SEQ ID NO: 229 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtcacatccggcgaaagccggctcccgcaaggggactcgtagaggtgctgc

SEQ ID NO: 230 - DNA, 249 bp, Unknown
  16S DNA Tag
cacgctgtaaacgatgggcactaggtgtgggaggtgtcgactcctcccgtgccgtagctaacgcattaagtgccccgcctggggagtacggccgcaaggctaaaactcaaaggaattgacggggacccgcacaagcagcggagcatgtggttcaattcgacgcaacgcgaagaaccttacctgggcttgacatgttggtgacagccgtggaaacacggtctcccttcggggcaccttcacaggtggtgc

SEQ ID NO: 231 - DNA, 248 bp, Unknown
  16S DNA Tag
ctagccctaaacgatgagtgcttggtgtggcgggtatcgatccctgccgtgccgaagctaacgcattaagcactccgcctggggagtacggtcgcaaggctgaaactcaaaggaattgacgggggcccgcacaagcggtggagcatgtggttcaattcgacgcaacgcgaagaaccttacccaggctcgaacggcagatgacatccggcgaaagtcggctcccgcaagggcatctgtcgaggtgctgc

SEQ ID NO: 232 - DNA, 252 bp, Unknown
  16S DNA Tag
cacgccgtaaacgatggatgctagccgttggcaggcttgcctgtcagtggcgcagctaacgcattaagcatcccgcctggggagtacggtcgcaagattaaaactcaaaggaattgacgggggcccgcacaagcggtggagcatgtggttcaattcgaagcaacgcgcagaaccttaccaacctttgacatgtcccgtatgagcaacagagatgtagctcttcagttcggctggcgggaacacaggtgctgc

SEQ ID NO: 233 - DNA, 249 bp, Unknown
  16S DNA Tag
cacgccgtaaacgatggatgctaggtgtgggcggtgtcgactccgtccgtgccgaagctaacgcattaagcatcccgcctggggagtacggccgcaaggctaaaactcaaaggaattgacgggggcccgcacaagcagcggagcatgctgtttaattcgaagcaacgcgaagaaccttacctgggcttgacatgtatccgaaatcctgggaaaccaggcccccagcaatggcggatatacaggtggtgc

SEQ ID NO: 234 - DNA, 251 bp, Unknown
  16S DNA Tag
ctagctataaacgatgttgactaggtgtcgacggtctaaaccccgccggtgccggagcaaacgcattaagtcaaccgcctggggactacggccgcaaggttaaaactcaaaggaattgacggggacccgcacaagcagcggagcgtgtggtttaattcgaggctacgcgaagaaccttaccagggattgacatgacggtagtagtgaagcgaaagcggaacgacccttcggggagccgtcacaggtgatgc

SEQ ID NO: 235 - DNA, 248 bp, Unknown
  16S DNA Tag
ctagccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtcacatccggcgaaagtcggctcccgcaaggggactcgtagaggtgctgc

SEQ ID NO: 236 - DNA, 249 bp, Unknown
  16S DNA Tag
cacgccgtaaacgatgggtgctaggtgtgggcggtgtcgactccgtccgtgccgaagctaacgcattaagcaccccgcctggggagtacggccgcaaggctaaaactcaaaggaattgacgggggcccgcacaagcagcggagcatgtggtttaattcgacgcgacgcgaagaaccttaccaaggcttgacatgaccaggaatgtcgtagaaatacggcagcccttcggggcctggccacaggtggtgc

SEQ ID NO: 237 - DNA, 251 bp, Unknown
  16S DNA Tag
cacgccgtaaacgatgggcactaggtgcttgggggagcgaccctctgagtgccggcgctaacgcattaagtgccccgcctggggagtacggccgcaaggctgaaactcaaaggaattgacgggggcccgcacaagcggtggagcatgtggtttaattcgacgcaacgcgaagaaccttacctaggcttgacatgcagaggaaagctttgagaaaccagagccctccttcgggacttctgcacaggtgctgc

SEQ ID NO: 238 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggtttaattcgacgcaacgcgaagaaccttacccaggcttgaactgcaggtgacatcctacgaaagtaggcttccgcaaggacacctgtagaggtgctgc

SEQ ID NO: 239 - DNA, 247 bp, Unknown
  16S DNA Tag
ctagccgtaaacgttgggtgctaggtgtgggcggatatcaaccccgtccgtgccgaagctaacgcattaagcaccccgcctggggagtacggccgcaaggctaaaactcaaaggaattgacggggccccgcacaagcggcggagcatgttgcttaattcgatgcgacgcgaagaaccttacctgggcttgacatgcagggtgaaacccgtgaaagcgggtgctcttcggagcctgcacaggtggtgc

SEQ ID NO: 240 - DNA, 251 bp, Unknown
  16S DNA Tag
ctagccgtaaacgatggacactaggtgttggtggtatcaaccccgccagtgccgaagctaacgcattaagtgtcccgcctggggagtacggccgcaaggctaaaactcaaaggaattgacgggggcccgcacaagcagcggagcgtggggtttaattcgacgcaacgcgaagaaccttaccagggcttgacatgaccaggaccgcggcggaaacgtcgctttcccgcaagggacctggccacaggtgttgc

SEQ ID NO: 241 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgaccactcctgaaagggagcttccgcaaggacactcgtagaggtgctgc

SEQ ID NO: 242 - DNA, 249 bp, Unknown
  16S DNA Tag
cacgccgtaaacgatgggtgctaggtgtgggcggtgtcgactccgtccgtgccgaagctaacgcattaagcaccccgcctggggagtacggccgcaaggctaaaactcaaaggaattgacgggggcccgcacaagcagcggagcatgttgtttaattcgacgcgacgcgaagaaccttaccaaggcttgacatgcacgggaatgtcgtagaaatacggcagcccttcggggctcgtgcacaggtggtgc

SEQ ID NO: 243 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaactgcgagtgaccacgcccgaaagggcgcttccgcaaggacactcgtagaggtgctgc

SEQ ID NO: 244 - DNA, 252 bp, Unknown
  16S DNA Tag
cacgccgtaaacgatggatgctagccgttggcgggtttactcgtcagtggcgcagctaacgcattaagcatcccgcctggggagtacggtcgcaagattaaaactcaaaggaattgacgggggcccgcacaagcggtggagcatgtggttcaattcgaagcaacgcgcagaaccttaccagcccttgacatgtcccgtatggacttcagagatgaggtccttcagttcggctggcgggaacacaggtgctgc

SEQ ID NO: 245 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaactgcaggtgacatcctacgaaagtaggtttccgcaaggacacctgtagaggtgctgc

SEQ ID NO: 246 - DNA, 253 bp, Unknown
  16S DNA Tag
ctggcagtaaacggtgcacgtttggtgtgggaggattcgaccccttccgcgccggagctaacgcgttaaacgtgccgcctggggagtacggtcgcaagattaaaactcaaagaaattgacgggggcccgcacaagcggtggagtatgtggcttaattcgatgcaacgcgaagaaccttaccaggccttgacatgcatttctaagttggtgaaagccagcgagtcccgaaagggacaacttgcacaggtgctgc

SEQ ID NO: 247 - DNA, 245 bp, Unknown
  16S DNA Tag
cacgccgtaaactatgggtgctagccgtcggccggcatgccggtcggtggcgcagcaaacgcattaagcaccccgcctggggagtacggtcgcaagattaaaactcaaaggaattgacgggggcccgcacaagcggtggagcatgtggtttaattcgaggcaacgcgaagaaccttaccagcccttgacttcccgatcggggctccggagacggagctcttcaattggtcggtgacaggtgctgc

SEQ ID NO: 248 - DNA, 248 bp, Unknown
  16S DNA Tag
ctagccctaaacgatgagtgcttggtgtggcgggtatcgatccctgccgtgccgaagctaacgcattaagcactccgcctggggagtacggtcgcaaggctgaaactcaaaggaattgacgggggcccgcacaagcggtggagcatgtggttcaattcgacgcaacgcgaagaaccttacccaggctcgaacggcgagtgacatccggcgaaagtcggcttccgcaaggacacttgtcgaggtgctgc

SEQ ID NO: 249 - DNA, 247 bp, Unknown
  16S DNA Tag
ccagctgtaaacgatgcagactcggtgatgggttggccttgtgccaacccagtgccgcagggaagccgttaagtctgccgcctggggagtacggtcgcaagactgaaacttaaaggaattggcgggggagcaccacaaggggtgaagcctgcggttcaattggagtcaacgccgggaatcttaccgggggcgacagcagtgtgaaggtcaagctgaagactttaccagacaagctgagaggaggtgc

SEQ ID NO: 250 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgacaaactacgaaagtggtcttccgcaaggacactcgtagaggtgctgc

SEQ ID NO: 251 - DNA, 249 bp, Unknown
  16S DNA Tag
ctagcagtaaacgatggccactaggtgtggggggtgttgaccccttccgtgccgtagctaacgcattaagtggcccgcctggggagtacggtcgcaagactaaaactcaaaggaattgacggggacccgcacaagcagcggagcgtgtggtttaattcgatgcaacgcgaagaaccttacctgggcttgacatgtagtcgcaacgcactgaaaggtgcgcctccgcaaggacggctacacagatgttgc

SEQ ID NO: 252 - DNA, 252 bp, Unknown
  16S DNA Tag
cacgctgtaaacgatgagtgctaagtgttagagggtttccgccctttagtgctgcagctaacgcattaagcactccgcctggggagtacggccgcaaggctgaaactcaaaggaattgacggggacccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 253 - DNA, 253 bp, Unknown
  16S DNA Tag
ctggcagtaaacggtgcacgtttggtgtgggaggattcgaccccttctgcgccggagctaacgcgttaaacgtgccgcctggggagtacggtcgcaagattaaaactcaaagaaattgacgggggcccgcacaagcggtggagtatgtggcttaattcgatgcaacgcgaagaaccttacctggccttgacatgcatctctaagctggtgaaagccagtgactcccgaaagggagaatttgcacaggtgctgc

SEQ ID NO: 254 - DNA, 252 bp, Unknown
  16S DNA Tag
cacgctgtaaacgatggatgctagccgttggcgagcttgctcgtcagtggcgcagctaacgctttaagcatcccgcctggggagtacggtcgcaagattaaaactcaaaggaattgacgggggcccgcacaagcggtggagcatgtggttcaattcgaagcaacgcgcagaaccttaccagcccttgacatcccggtcgcggagagcagagacgcactccttcagttcggctggaccggagacaggtgctgc

SEQ ID NO: 255 - DNA, 253 bp, Unknown
  16S DNA Tag
ctggcagtaaacggtgcacgtttggtgtgggaggattcgaccccttctgcgccggagctaacgcgttaaacgtgccgcctggggagtacggtcgcaagattaaaactcaaagaaattgacgggggcccgcacaagcggtggagtatgtggctcaattcgatgcaacgcgaagaaccttaccaggccttgacatgcatctctaagttggtgaaagccagtgagtggggaaacccacaatttgcacaggtgctgc

SEQ ID NO: 256 - DNA, 248 bp, Unknown
  16S DNA Tag
ctggccctaaacgatgagtgcttggtgtggcgggtatcgatccctgccgtgccgaagctaacgcattaagcactccgcctggggagtacggtcgcaaggctgaaactcaaaggaattgacgggggcccgcacaagcggtggagcatgtggttcaattcgacgcaacgcgaagaaccttacccaggctcgaacggtagctgacatcccacgaaagtgggctcccgcaagggcagctgtcgaggtgctgc

SEQ ID NO: 257 - DNA, 249 bp, Unknown
  16S DNA Tag
cacgccgtaaacgatgggtgctaggtgtgggcggtgtcgactccgtccgtgccgaagctaacgcattaagcaccccgcctggggagtacggccgcaaggctaaaactcaaaggaattgacgggggcccgcacaagcagcggagcatgtggttcaattcgacgcaacgcgaagaaccttaccgagacttgacatgcactggaatgtcgtagaaatacggcagcccttcggggctagtgcacaggtggtgc

SEQ ID NO: 258 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgatcagcgacgaaagttgccttccgcaaggacactcgtagaggtgctgc

SEQ ID NO: 259 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggcttgaacagcgagtgaccacttccgaaaggaagcttccgcaaggacactcgtagaggtgctgc

SEQ ID NO: 260 - DNA, 248 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggtttaattcgacgcaacgcgaagaaccttacccaggctcgaacggcatctgacatcctacgaaagtaggctcccgcaagggcagatgtcgaggtgctgc

SEQ ID NO: 261 - DNA, 253 bp, Unknown
  16S DNA Tag
cacgccgtaaacgatgagtgctagacgttgggaggcctagccttccggtgtcgcagctaacgcattaagcactccgcctggggagtacggccgcaaggttaaaactcaaaggaattgacgggggcccgcacaagcggtggagcatgtggtttaattcgaagcaacgcgcagaaccttaccagcccttgacatggggagtgtgagcctgggagaccgggttcttcagttcggctggctcccgcacaggtgctgc

SEQ ID NO: 262 - DNA, 249 bp, Unknown
  16S DNA Tag
cacgccgtaaacgatgggtgctaggtgtgggcggtgtcgactccgtccgtgccgtagctaacgcattaagcaccccgcctggggagtacggccgcaaggctaaaactcaaaggaattgacggggacccgcacaagcagcggagcatgttgtttaattcgacgcgacgcgaagaaccttaccaaggcttgacatgcacgggaatgtcgtagaaatacggcagcccttcggggctcgtgcacaggtggtgc

SEQ ID NO: 263 - DNA, 248 bp, Unknown
  16S DNA Tag
cacgccgtaaacgttgggcactaggtgtggggctctatcgacgggttccgtgccgtagctaacgcattaagtgccccgcctggggagtacggccgcaaggctaaaactcaaaggaattgacgggggcccgcacaagcggcggagcgtgtggctcaattcgatgcaacgcgaagaaccttacctgggcttgacatgtacggtaaagcgggtgaaagcccgtgtgcttcggcgccgtacacaggtggtgc

SEQ ID NO: 264 - DNA, 255 bp, Unknown
  16S DNA Tag
ctagccttaaacgatgaatgcttggtgtctggggttttatagtccccgggtgccgtcgctaacgctttaagcattccgcctggggagtacggtcgcaagactgaaactcaaaggaattgacggggacccgcacaagcggtggagcatgtggtttaattcgacgcaacgcgaagaaccttacctgggctagaatgcctctgaccggcgtagagatacgctttcctgggtaaaaccaggcagagtgcaaggtgctgc

SEQ ID NO: 265 - DNA, 249 bp, Unknown
  16S DNA Tag
cacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgactgcagagatgtggtttcccttgtggcctgtgtgcaggtggtgc

SEQ ID NO: 266 - DNA, 251 bp, Unknown
  16S DNA Tag
cacgccgtaaacgatgaatgccagccgttagtgggtttactcactagtggcgcagctaacgctttaagcattccgcctggggagtacggtcgcaagattaaaactcaaaggaattgacgggggcccgcacaagcggtggagcatgtggttttaattcgacgcaacgcgcagaaccttaccagcccttgacatgtccaggaccggtcgcagagatgtgaccttctcttcggagcctggaacacaggtgctgc

SEQ ID NO: 267 - DNA, 253 bp, Unknown
  16S DNA Tag
cacgccgtaaacgatgaatgccagccgttagtgggtttactcactagtggcgcagctaacgctttaagcattccgcctggggagtacggtcgcaagattaaaactcaaaggaattgacgggggcccgcacaagcggtggagcatgtggttttaattcgacgcaacgcgcagaaccttaccagcccttgacatcccggtcgcggactccagagacggagttcttcagttcggctggaccggagacaggtgctgc

SEQ ID NO: 268 - DNA, 247 bp, Unknown
  16S DNA Tag
cacgccgtaaactatgggtgctagccgtcggtcggcatgccggtcggtggcgcagcaaacgcattaagcaccccgcctggggagtacggtcgcaagattaaaactcaaaggaattgacgggggcccgcacaagcggtggagcatgtggtttttaattcgaggcaacgcgaagaaccttaccagcccttgacttcccgatcggggctccggagacggagctcttcaattggtcggtgacaggtgctgc

SEQ ID NO: 269 - DNA, 250 bp, Unknown
  16S DNA Tag
cacgccgtaaacgatgggtgctaggtgtgggcggtgtcgactccgtccgtgccgaagctaacgcattaagcaccccgcctggggagtacggccgcaaggctaaaactcaaaggaattgacgggggcccgcacaagcagcggagcatgtggttttaattcgacgcaacgcgaagaaccttaccagggcttgacatgcactggaatgtcggagaaattcggcagcccttcggggccagtgcacaggtggtgc

SEQ ID NO: 270 - DNA, 246 bp, Unknown
  16S DNA Tag
cacgccgtaaactatgggtgctagccgtcggggagcatgctcttcggtggcgcagcaaacgcattaagcaccccgcctggggagtacggtcgcaagattaaaactcaaaggaattgacgggggcccgcacaagcggtggagcatgtggttttaattcgaggcaacgcgaagaaccttaccagcccttgacttcccgatcgggggaccagagatggacctcttcaattggtcggtgacaggtgctgc

SEQ ID NO: 271 - DNA, 246 bp, Unknown
  16S DNA Tag
cacgccgtaaactatgggtgctagccgtcggtcggcatgccggtcggtggcgcagcaaacgcattaagcaccccgcctggggagtacggtcgcaagattaaaactcaaaggaattgacgggggcccgcacaagcggtggagcatgtggttttaattcgaggcaacgcgaagaaccttaccagcccttgacttcccgatcggggctccggagacggagctcttcaattggtcggtgacaggtgctgc

SEQ ID NO: 272 - DNA, 252 bp, Unknown
  16S DNA Tag
cacgccgtaaacgatgaatgccagccgttagtgggtttactcactagtggcgcagctaacgctttaagcattccgcctggggagtacggtcgcaagattaaaactcaaaggaattgacgggggcccgcacaagcggtggagcatgtggtttttaattcgacgcaacgcgcagaaccttaccagcccttgacatgtccaggaccggtcgcagagatgtgaccttctcttcggagcctggaacacaggtgctgc

SEQ ID NO: 273 - DNA, 257 bp, Unknown
  16S DNA Tag
ctagccttaaacgatgctgacttggtgttccaggtttttagtcctggggtgccggagctaacgcgttaagtcagccgcctggggagtacggtcgcaaggctgaaactcaaaggaattgacggggacccgcacaagcggtggagcatgtggtttttaattcgacgcaacgcgaagaaccttacctgggctagaatgcggtggataagccctagagatagggcgatccgggtaaccggctcctgcctgcaaggtgctgc

SEQ ID NO: 274 - DNA, 248 bp, Unknown
  16S DNA Tag
ctagccgtaaacgttgggtgctaggtgtgggtggacatcgaccccatccgtgccgaagctaacgcattaagcaccccgcctggggagtacggccgcaaggctaaaactcaaaggaattgacgggggcccgcacaagcggcggagcatgcggctttaattcgatgcgacgcgaagaaccttacctgggcttgacatgcagggcaaagccggtgaaagccggcgctctacggagcctgcacaggtggtgc

SEQ ID NO: 275 - DNA, 248 bp, Unknown
  16S DNA Tag
cacgctgtaaacgttgggcgctaggtgtggggtccttccacggattccgtgccgcagctaacgcattaagcgccccgcctggggagtacggccgcaaggctaaaactcaaaggaattgacgggggcccgcacaagcggcggagcatgtttgcttaattcgacgcgacgcgaagaaccttaccaaggtttgacatgcagggaaaagccgtagagatacggtgtccgtaagggccctgcacaggtggtgc

SEQ ID NO: 276 - DNA, 252 bp, Unknown
  16S DNA Tag
cacgccgtaaacgatgggtgctaggtgtggggggtgtcgactccctccgtgccgaagctaacgcattaagcaccccgcctggggagtacggccgcaaggctaaaactcaaaggaattgacgggggcccgcacaagcagcggagcatgtttgtttttaattcgacgcgacgcgaagaaccttaccaaggcttgacatgcacgggaatgtcgtggaaacacggcagcccttcggggctcgtgcacaggtggtgc

SEQ ID NO: 277 - DNA, 249 bp, Unknown
  16S DNA Tag
ctagccttaaacgatgtgcacttggtgttgggggtatcgacccctccagtgccgaagctaacgcattaagtgcaccgcctggggagtacggtcgcaaggctgaaactcaaaggaattgacgggggcccgcacaagcggtggagcatgtggtttaattcgacgcaacgcgaagaaccttaccggggtttgaactgccggtgacagctccagaaatggagtcttccttcgggacgccggcagaggtgctgc

SEQ ID NO: 278 - DNA, 248 bp, Unknown
  16S DNA Tag
cacgccgtaaacgttgggcgctaggtgtgggatccttccacggattccgtgccgcagctaacgcattaagcgccccgcctggggagtacggccgcaaggctaaaactcaaaggaattgacgggggcccgcacaagcggcggagcatgtttgcttaattcgacgcaacgcgaagaaccttaccaaggtttgacatgcagggaaaagccgtagagatacggtgtccgtaagggtcctgcacaggtggtgc

SEQ ID NO: 279 - DNA, 249 bp, Unknown
  16S DNA Tag
ctggccctaaacgatgaatgcttggtgtgacgggtatcgatccctgtcgtgccgcagttaacgcgttaagcattccgcctggggagtacggtcgcaaggctgaaactcaaaggaattgacgggggcccgcacaagcggtggagcatgtggttcaatttcgacgcaacgcgaagaaccttacctgggctcgaagtgcagatgacactcggcgaaagccgattcccgcaagggcatttgcagaggtgctgc

SEQ ID NO: 280 - DNA, 255 bp, Unknown
  16S DNA Tag
cacgctgtaaacgatgagtgctaagtgttagagggtttccgccctttagtgctgcagctaacgcattaagcactccgcctggggagtacggccgcaaggctgaaactcaaaggaattgacggggacccgcacaagcggtggagcatgtggtttttaatttcgaagcaacgcgaagaaccttaccaggtcttgacatcctctgacactcctagagataggacgttccccttcgggggacagagtgacaggtggtgc

SEQ ID NO: 281 - DNA, 253 bp, Unknown
  16S DNA Tag
cacgctgtaaacgatggatgctagccgttggcgagcttgctcgtcagtggcgcagctaacgctttaagcatcccgcctggggagtacggtcgcaagattaaaactcaaaggaattgacgggggcccgcacaagcggtggagcatgtggtttcaattcgaagcaacgcgcagaaccttaccagcccttgacatcccggtcgcggagagcggagacgcactccttcagttcggctggaccggagacaggtgctgc

SEQ ID NO: 282 - DNA, 249 bp, Unknown
  16S DNA Tag
ctggccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaatttcgacgcaacgcgaagaaccttacccaggcttgaactgcaggtgacatcctacgaaagtaggcttccgcaaggacacctgtagaggtgctgc

SEQ ID NO: 283 - DNA, 248 bp, Unknown
  16S DNA Tag
cacgccgtaaacgttgggcgctaggtgtgggatccttccacggattccgtgccgcagctaacgcattaagcgccccgcctggggagtacggccgcaaggctaaaactcaaaggaattgacgggggcccgcacaagcggcggagcatgtttgcttaattcgacgcaacgcgaagaaccttaccaaggtttgacatgcagggaaaagccgtagagatatggtgtccgtaagggtcctgcacaggtggtgc

SEQ ID NO: 284 - DNA, 257 bp, Unknown
  16S DNA Tag
ctggccctaaacgatgtccactagattacggcgcctttgacggcgtcgtggtcgaagcaaaagtgttaagtggaccgcctgggaagtacggtcgcaaggctaaaactcaaaggaattgacgggggctcacacaagcggtggagcatgtggttcaattcgaagcaacgcgcagaaccttacctgggcttgacatgcttggattacctcggtgaaagccgagtaggccccgcaaggggtacaacaagtacaggtgctgc

SEQ ID NO: 285 - DNA, 249 bp, Unknown
  16S DNA Tag
cacgccgtaaactatgcttgctagctgttatcagtatcgaccctggtagtagcgaagctaacgcgttaagcaagccgcctgtggagtacgagcgcaagcttaaaacataaaggaattgacggggacccgcacaagcggtggagcgtgttgtttaattcgacggtaagcgaagaaccttacccaggcttgacatcctgccaaactctccgaaaggagagtgtgccttcgggccgcagtgacaggtgatgc

SEQ ID NO: 286 - DNA, 249 bp, Unknown
  16S DNA Tag
cacgccctaaacggtgcacactaggtcttggcggattcgaccccaccagggcccaagctaacgcgttaagtgtgccgcctgaggactacggccgcaaggctaaaactcaaaggaattgacgggggcccgcacaagcggtggagtatgtggctcaattcgatgcaacgcgaagaaccttacctggccttgacatgcactagacagacgctgaaaggcgttctcccttcggggctggtgcacaggtgctgc

SEQ ID NO: 287 - DNA, 248 bp, Unknown
  16S DNA Tag
cacgccgtaaacgatgagtgctaagtgttgggggtcgaacctcagtgctgaagttaacgcattaagcactccgcctggggagtacggtcgcaagactgaaactcaaaggaattgacggggacccgcacaagcggtggagcatgtggtttaattcgaagcaacgcgaagaaccttaccaggtcttgacatactgatgaccgtcctagagataggaaatctccttcgggagcatcagatacaggtggtgc

SEQ ID NO: 288 - DNA, 258 bp, Unknown
  16S DNA Tag
ctagcagtaaacggtgcacgtttggtgtgggaggattcgaccccttccgtgccggagctaacgcgttaaacgtgccgcctgggaagtacggtcgcaagattaaaactcaaagaaattgacgggggcccgcacaagcggtggagtatgtggcttaattcgatgcaacgcgaagaaccttacctggccttgacatgtatcgtgacaatctgtgaaagcaggtgactacccgcaagggtgatggggatacacaggtgctgc

SEQ ID NO: 289 - DNA, 257 bp, Unknown
  16S DNA Tag
ctggccctaaacgatgtccactagattgcggcgggtttgaccccgtcgcggtcgaagcaaaagtgttaagtggaccgcctgggaagtacggtcgcaaggctaaaactcaaaggaattgacgggggctcacacaagcggtggagcatgtggttcaattcgaagcaacgcgcagaaccttacctgggtttgacatgcttggattatccttatgaaagtaaggtaggccccgcaaggggtacaacaagcacaggtgctgc

SEQ ID NO: 290 - DNA, 251 bp, Unknown
  16S DNA Tag
cacgccgtaaacgatgggtactaggtgcttgggggagcgaccccctgagtgccggcgctaacgcattaagtaccccgcctggggagtacggccgcaaggctgaaactcaaaggaattgacgggggcccgcacaagcggtggagcatgtggtttaattcgacgcaacgcgaagaaccttacctaggcttgacatgtatgggaaacgcttgagaaaccaggcgcctccttcgggacccatgcacaggtgctgc

SEQ ID NO: 291 - DNA, 264 bp, Unknown
  16S DNA Tag
catgccgtaaacgatgagtgttcgcccttggtctgtctacgctacgctacgctacgcggatcaggggcccagctaacgcgtgaaacactccgcctggggagtacggtcgcaagaccgaaactcaaaggaattgacgggggcctgcacaagcggtggagcatgtggtttaattcgatacaacgcgcaaaaccttaccagcccttgacatatgaacaacaaaacctgtctttaacgggatggtacttactttcatacaggtgctgc

SEQ ID NO: 292 - DNA, 248 bp, Unknown
  16S DNA Tag
cacgccgtaaacgttgggcgctaggtgtggggctcattccacgagttccgtgccgcagctaacgcattaagcgccccgcctggggagtacggccgcaaggctaaaactcaaaggaattgacgggggcccgcacaagcggcggagcatgttgcttaattcgatgcaacgcgaagaaccttacctaggcttgacatgcacggaaatctcgcagagatgcggggtccgtaagggccgtgcacaggtggtgc

SEQ ID NO: 293 - DNA, 249 bp, Unknown; identical to SEQ ID NO: 183
  16S DNA Tag

SEQ ID NO: 294 - DNA, 247 bp, Unknown
  16S DNA Tag
cacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgc

SEQ ID NO: 295 - DNA, 249 bp, Unknown
  16S DNA Tag
cacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccggagacggtcgcccccttgtggtcggtgtacaggtggtgc

SEQ ID NO: 296 - DNA, 244 bp, Unknown
  16S DNA Tag
cacgccctaaacgatgaatactcgatgttagcgatatacggttagcgtctaagcgaaagcgttaagtattccacctggggagtacgcccgcaagggtgaaactcaaaggaattgacgggggcccgcacaagcggaggagcatgtggtttaattcgatgatacgcgaggaaccttacccgggcttgaaagttactgaagacagcagagacgctgtcgtccttcgggacaggaaactaggtgctgc

SEQ ID NO: 297 - DNA, 248 bp, Unknown
  16S DNA Tag
cacgccgtaaacggtgggtgctaggtgtggggggcttccacgccctctgtgccgcagctaacgcattaagcaccccgcctggggagtacggccgcaaggctaaaactcaaaggaattgacgggggcccgcacaagcggcggagcatgtggattaattcgatgcaacgcgaagaaccttacctgggcttgacatgcactggaaaccagtagagatattggcccccttgtggccggtgtacaggtggtgc

SEQ ID NO: 298 - DNA, 248 bp, Unknown
  16S DNA Tag
cacgccgtaaacgttgggcgctaggtgtggggttcttccacggattccgcgccgtagctaacgcattaagcgccccgcctggggagtacggccgcaaggctaaaactcaaaggaattgacgggggcccgcacaagcggcggagcatgttgcttaattcgacgcaacgcgaagaaccttaccaaggcttgacatcgccggaaaaccatcagagatggtgggtcctttttgggccggtgacaggtggtgc

SEQ ID NO: 299 - DNA, 250 bp, Unknown
  16S DNA Tag
cacgccgtaaacgatgaatgctaggtgttaggggtttcaatacccttggtgccgaagttaacacattaagcattccgcctggggagtacgctcgcaagagtgaaactcaaaggaattgacggggacccgcacaagcagtggagtatgtggtttaattcgaagcaacgcgaagaaccttaccaggtcttgacatccctctgaccggtctggagacaggccttcccttcggggcagaggagacaggtggtgc

SEQ ID NO: 300 - DNA, 250 bp, Unknown
  16S DNA Tag
cacgccctaaacgatgtcaactggttgttggacggcttgctgttcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgcctggaaccctgcagagatgtgggggtgctcgaaagagaaccaggacacaggtgctgc

SEQ ID NO: 301 - DNA, 248 bp, Unknown
  16S DNA Tag
cacgctgtaaacgttgggcgctaggtgtgggcgacatccacgttgtccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggcttgacatgcgccagacatccccagagatggggcttcccttgtggttggtgtacaggtggtgc

SEQ ID NO: 302 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaaaccctggagacagggtcccccttgtggtcggtgtacaggtggtgc

SEQ ID NO: 303 - DNA, 249 bp, Unknown; identical to SEQ ID NO: 20
  16S DNA Tag

SEQ ID NO: 304 - DNA, 249 bp, Unknown
  16S DNA Tag
cacgccgtaaacgatgggcactaggtgttgggggtgtcgactcccccggcgccgtagctaacgcattaagtgccccgcctggggagtacggccgcaaggctaaaactcaaaggaattgacgggggcccgcacaagcagcggagcatgtggtttaattcgacgcaacgcgaagaaccttacctgggcttgacatgttcgtgacaggtgtggaaacacaccctcccttcggggcacgatcacaggtggtgc

SEQ ID NO: 305 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacgccggaaaaccctggagacagggtcccccttgtggtcggtgtacaggtggtgc

SEQ ID NO: 306 - DNA, 247 bp, Unknown
  16S DNA Tag
ccagctgtaaacgatgcagactcggtgatgaataggcttcgtgcctattcagtgccgcagggaagccgttaagtctgccgcctggggagtacggtcgcaagactgaaacttaaaggaattggcgggggagcaccacaaggggtgaagcctgcggttcaattggagtcaacgccgggaatcttaccgggggcgacagcagagtgaaggtcaagctgaagactttaccagacaagctgagaggaggtgc

SEQ ID NO: 307 - DNA, 252 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgc

SEQ ID NO: 308 - DNA, 252 bp, Unknown
  16S DNA Tag
cacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgc

SEQ ID NO: 309 - DNA, 252 bp, Unknown
  16S DNA Tag
cacgccctaaacgatgcgaactggatgttggtctcaactcggagatcagtgtcgaagctaacgcgttaagttcgccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgtttcccttcggggaacagagtgacaggtggtgc

SEQ ID NO: 310 - DNA, 252 bp, Unknown
  16S DNA Tag
cacgccgtaaacgatgagtgctaagtgttagagggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 311 - DNA, 252 bp, Unknown
  16S DNA Tag
cacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 312 - DNA, 252 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcccactgcccggtatagagatatacctttcccttcggggacagtggtgacaggtggtgc

SEQ ID NO: 313 - DNA, 252 bp, Unknown
  16S DNA Tag
cacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgtttcccttcggggaacagagtgacaggtggtgc

SEQ ID NO: 314 - DNA, 252 bp, Unknown
  16S DNA Tag
cacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcgttccccttcgggggacagagtgacaggtggtgc

SEQ ID NO: 315 - DNA, 252 bp, Unknown
  16S DNA Tag
cacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgc

SEQ ID NO: 316 - DNA, 253 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 317 - DNA, 252 bp, Unknown
  16S DNA Tag
cacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcccactgcccggtatagagatatacctttcccttcggggacagtggtgacaggtggtgc

SEQ ID NO: 318 - DNA, 252 bp, Unknown
  16S DNA Tag
cacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacactcctagagataggacgtttcccttcggggaacagagtgacaggtggtgc

SEQ ID NO: 319 - DNA, 252 bp, Unknown
  16S DNA Tag
cacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgc

SEQ ID NO: 320 - DNA, 249 bp, Unknown
  16S DNA Tag
cacgccataaacgatgacaactagacgttgggaacatatggttcacagtgtcgcagctaacgcgttaagttgtccgcctggggagtacggccgcaaggttgaaactcaaaggaattgacggggacccgcacaagcggtggagcatgtggtttaattcgatgcaacgcgaagaaccttacctggtcttgacatccacggaagcccgaagagatttgggtgtgccgcaaggaaccgtgagacaggtgctgc

SEQ ID NO: 321 - DNA, 252 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcccactgaccggtatagagatatacctttcccttcggggacagtggtgacaggtggtgc

SEQ ID NO: 322 - DNA, 252 bp, Unknown
  16S DNA Tag
cacgccgtaaacgatgagtgctaagtgttagggggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 323 - DNA, 252 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacggggacccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 324 - DNA, 252 bp, Unknown
  16S DNA Tag
cacgccgtaaacgatgagtgctaagtgttagagggtttccgccctttagtgctgcagcaaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 325 - DNA, 252 bp, Unknown
  16S DNA Tag
cacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggaatttccccttcgggggacagagtgacaggtggtgc

SEQ ID NO: 326 - DNA, 252 bp, Unknown
  16S DNA Tag
cacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcccactgaccggtatagagatatacctttcccttcggggacagtggtgacaggtggtgc

SEQ ID NO: 327 - DNA, 252 bp, Unknown
  16S DNA Tag
cacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccactgcccggtatagagatatacctttcccttcggggacagtggtgacaggtggtgc

SEQ ID NO: 328 - DNA, 250 bp, Unknown
  16S DNA Tag
cacgccctaaacgatgcgaactggatgttggtctcaactcggagatcagtgtcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatctgtcgaatcctgcagagatgcgggagtgccgcaaggaacgacaagacaggtgctgc

SEQ ID NO: 329 - DNA, 247 bp, Unknown
  16S DNA Tag
catgccgtaaacgttgggcgctaggtgtggggaccttccacggtttccgtgccgcagctaacgcattaagcgccccgcctggggagtacggccgcaaggctaaaactcaaaggaattgacgggggcccgcacaagcggcggagcatgctgattaattcgatgcaacgcgaagaaccttacctaggtttgacatatagagaaaacttacagagatgtaaggtccttttgggctctatacaggtggtgc

SEQ ID NO: 330 - DNA, 252 bp, Unknown
  16S DNA Tag
cacgccgtaaacgatgaatgttagccgtcgggcagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcccgatcgcggttaccggagacggtatccttcagttcggctggatcggagacaggtgctgc

SEQ ID NO: 331 - DNA, 253 bp, Unknown
  16S DNA Tag
cacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcccactgcccggtatagagatatacctttcccttttagggacagtggtgacaggtggtgc

SEQ ID NO: 332 - DNA, 250 bp, Unknown
  16S DNA Tag
cacgccctaaacgatgcgaactggatgttgggtgcaactaggcactcagtatcgaagctaacgcgttaagttcgccgcctggggagtacggtcgcaagactgaaactcaaaggaattgacgggggcccgcacaagcggtggagtatgtggtttaattcgatgcaacgcgcagaaccttacctggccttgacatccacggaaccctgcagagatgcgggggtgccttcgggaaccgtgagacaggtgctgc

SEQ ID NO: 333 - DNA, 253 bp, Unknown
  16S DNA Tag
ctggcagtaaacggtgcgcgtttggtgtgggaggattcgaccccttccgtgccggagctaacgcgttaaacgcgccgcctggggagtacggtcgcaagattaaaactcaaagaaattgacgggggcccgcacaagcggtggagtatgtggcttaattcgatgcaacgcgaagaaccttacctgggcttgacatgcatctctaagtcggtgaaagccggcgagtcccgcaagggacaatttgcacaggtgctgc

SEQ ID NO: 334 - DNA, 247 bp, Unknown
  16S DNA Tag
ctggccgtaaacgatgtgtgctggatggtggggggtaaaaccgtcactgtcgaagctaacgcgataagcacaccgcctggggagtacggccgcaaggctaaaactcaaaggaattgacgggggcccgcacaagcagcggagcgtgtggtttaattcgaggcaacgcgaagaaccttacctgggcttgacatactagtggtagggaaccgaaaggggaccgaccttcgggagctagtacaggtgctgc

SEQ ID NO: 335 - DNA, 244 bp, Unknown
  16S DNA Tag
cacactgtaaacgttgattactcgctgtgtgcgatatacagtacgcggccaagcgaaagcgataagtaatccacctggggagtacgccggcaacggtgaaactcaaaggaattgacgggggtccgcacaagcggtggagcatgtggtttaattcgatgatacgcgaggaaccttacctggactagaatgcccttgaccggttcagagatggacttttcagcaatgacaaggagcaaggtgctgc

SEQ ID NO: 336 - DNA, 252 bp, Unknown
  16S DNA Tag
cacgccgtaaacgatgagtgctaggtgtcacgggctttgaccctcgtggtgccgaagctaacgcgttaagcactccgcctggggagtacggccgcaaggctaaaactcaaaggaattgacgggggcccgcacaagcggtggagcatgttgtttaattcgacgcaacgcgaagaaccttacctgggctagaaagtacgagaatccgagcgaaagttcggagtgctcttcggagaactcgtggttaggtgctgc

SEQ ID NO: 337 - DNA, 249 bp, Unknown
  16S DNA Tag
cacgccataaacgatgaggactagacgttggaggctttaggctttcagtgtcgtagctaacgcgctaagtcctccgcctggggagtacggccgcaaggttgaaactcaaaggaattgacggggacccgcacaagcggtggagcatgtggtttaattcgatgcaacgcgaagaaccttacctggtcttgacatccatggaatcccgcagagatgtgggagtgccgtaaggaaccatgagacaggtgctgc

SEQ ID NO: 338 - DNA, 250 bp, Unknown
  16S DNA Tag
cacgccgtaaactatggatgctagctgttagaagtatcgacccttctagtagcgaagctaacgcgttaagcatcccgcctgtggagtacggtcgcaagactaaaacataaaggaattgacggggacccgcacaagcggtggagcgtgttgtttaattcgatggtaagcgaagaaccttacccaggcttgacatccttggaaagcatccgaaaggatactgtgccctcgggagccaagtgacaggtgatgc

SEQ ID NO: 339 - DNA, 244 bp, Unknown
  16S DNA Tag
cacactgtaaacgttgattactcgatgttggcgatatactgtcagcgtcttagcgcaagcgataagtaatccacctggggagtacgccggcaacggtgaaactcaaaggaattgacgggggtccgcacaagcggtggagcatgtggtttaattcgatgatacgcgaggaaccttacctggactagaatgcccttgatggatacagagatgtatcgttccgcaaggacaaggagcaaggtgctgc

SEQ ID NO: 340 - DNA, 250 bp, Unknown
  16S DNA Tag
cacgccgtaaactatggatgctagctgttagaagtatcgacccttctagtagcgaagctaacgcgttaagcatcccgcctgtggagtacggtcgcaagactaaaacataaaggaattgacggggacccgcacaagcggtggagcgtgttgtttaattcgatggtaagcgaagaaccttacccaggcttgacatccttggaaggcttccgaaaggaaactgtgccctcgggaaccaagtgacaggtgatgc

SEQ ID NO: 341 - DNA, 249 bp, Unknown
  16S DNA Tag
cacgccgtaaactatggatgctagctgttagaagtatcgacccttctagtagcgaagctaacgcgttaagcatcccgcctgtggagtacggtcgcaagactaaaacataaaggaattgacggggacccgcacaagcggtggagcgtgttgtttaattcgatggtaagcgaagaaccttacccaggcttgacatccttggaatttctgcgaaagcagagagtgcctttggaaccaagtgacaggtgttgc

SEQ ID NO: 342 - DNA, 251 bp, Unknown
  16S DNA Tag
cacgccgtaaacgatgagtgctagatgctgtgggtattgacccccgcggtgtcgcagccaacgcgttaagcactccgcctggggagtacggccgcaaggctaaaactcaaaggaattgacgggggcccgcacaagcggtggagcatgtggttcaattcgacgcaacgcgcagaaccttacctgggttaaatccactagaaggctacagagatgtggctttgcccttcggggagctagtgagaaggtgctgc

SEQ ID NO: 343 - DNA, 249 bp, Unknown
  16S DNA Tag
cacgccgtaaacgttgggcgctagatgtggggacctttccacggtttccgtgtcgtagctaacgcattaagcgccccgcctggggagtacggccgcaaggctaaaactcaaaggaattgacggggcccgcacaagcggcggagcatgcggattaattcgatgcaacgcgaagaaccttaccaaggcttgacataaccgagaacgccgcagaaatgtggaactctttggacactcggttacaggtggtgc

SEQ ID NO: 344 - DNA, 252 bp, Unknown
  16S DNA Tag
ctggcagtaaacggtgcgcgtttggtgtgggaggaatcgaccccttccgtgccggagctaacgcgttaaacgcgccgcctggggagtacggtcgcaagattaaaactcaaagaaattgacggggcccgcacaagcggtggagtatgtggcttaattcgatgcaacgcgaagaaccttacctaggcttgacatgcatctctaagtcggtgaaagccggcgagtaccgcaaggtacaatttgcacaggtgctgc

SEQ ID NO: 345 - DNA, 249 bp, Unknown
  16S DNA Tag
cacgccgtaaacgttgggcgctagatgtggggacctttccacggtttccgtgtcgtagctaacgcattaagcgccccgcctggggagtacggccgcaaggctaaaactcaaaggaattgacggggcccgcacaagcggcggagcatgcggattaattcgatgcaacgcgaagaaccttaccaaggcttgacataaccgagaacgccctagaaatagggaactctttggacactcggttacaggtggtgc

SEQ ID NO: 346 - DNA, 251 bp, Unknown
  16S DNA Tag
ctagccgtaaacgatgtgaacttggcgttggtgggttaaactccatcagtgccgtagcaaacgcgataagttcaccgcctggggactacggccgcaaggttaaaactcaaaggaattgacgggggcccgcacaagcagcggagcgtgtggtttaattcgatgctacacgaagaaccttacccaggcttgacatacaggtggtagggatccgaaaggtgaccgacccgcaagggagcctgtacaggtgttgc

SEQ ID NO: 347 - DNA, 251 bp, Unknown
  16S DNA Tag
ctagccgtaaacgatgtgaacttggcgttggtgggttaaactccatcagtgccgaagcaaacgcgataagttcaccgcctggggactacggccgcaaggttaaaactcaaaggaattgacgggggcccgcacaagcagcggagcgtgtggtttaattcgatgctacacgaagaaccttacccaggcttgacatacaggtggtagggatccgaaaggtgaccgacccgcaagggagcctgaacaggtgttgc

SEQ ID NO: 348 - DNA, 248 bp, Unknown
  16S DNA Tag
cacgccgtaaacgttgggaactaggtgttggcgacattccacgtcgtcggtgccgcagctaacgcattaagttccccgcctggggagtacggccgcaaggctaaaactcaaaggaattgacggggcccgcacaagcagcggagcatgtggcttaattcgacgcaacgcgaagaaccttaccaaggcttgacatataccggaaagcatcagagatggtgccccccttgtggtcggtatacaggtggtgc

SEQ ID NO: 349 - DNA, 252 bp, Unknown
  16S DNA Tag
ctggccctaaacgatgaatacttggtgtctggagtttcaatactccgggtgccgtcgctaacgttttaagtattccgcctggggagtacgcacgcaagtgtgaaactcaaaggaattgacggggacccgcacaagcggtggagcatgtggtttaattcgacgcaacgcgaagaaccttacctaggctagaatgtgagggaattctgggtaatgccagaagtccgggaaaccggacccaaaacaaggtgctgc

SEQ ID NO: 350 - DNA, 248 bp, Unknown
  16S DNA Tag
cacgccgtaaacgatgaatgttagccgtcgggcagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatgcccggctagctacagagatgtagtgttcccttcggggaccgggacacaggtgctgc

SEQ ID NO: 351 - DNA, 244 bp, Unknown
  16S DNA Tag
cacgctgtaaacgatgattactcgatgttggcgataaactgtcagcgtcttagcgaaagcgttaagtaatccacctggggagtacgcccgcaagggtgaaactcaaaggaattgacgggggcccgcacaagcggtggagcatgtggtttaattcgatgatacgcgaggaaccttacctaggctagaatgcgcgtgaccggtccagagatggacctttccttcgggacacaaagcaaggtgctgc

SEQ ID NO: 352 - DNA, 249 bp, Unknown
  16S DNA Tag
cacgccataaacgatgagaactggatgtcggaggggtctgcccttcggtgtcgtagctaacgcgttaagttctccgcctggggagtacggccgcaaggttgaaactcaaaggaattgacggggacccgcacaagcggtggagcatgtggtttaattcgatgcaacgcgaagaaccttacctggtcttgacatcctaggaacctcgcagagatgtgggggtgccttcgggaacctggagacaggtgctgc

SEQ ID NO: 353 - DNA, 250 bp, Unknown
  16S DNA Tag
cacgccgtaaacgatgaatgctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtacagagatgtacctttccttcgggacagaggagacaggtggtgc

SEQ ID NO: 354 - DNA, 247 bp, Unknown
  16S DNA Tag
cacgccctaaacgatggatactcgacatcagcgatacactgttggtgtctgagcgaaagcattaagtatcccacctgggaagtacgaccgcaaggttgaaactcaaaggaattgacgggggtccgcacaagcggtggagcatgtggtttaattcgatgatacgcgaggaaccttacctgggctagaatgtcgggggaatatgggtgaaagctcatagtgtagcaatacaccgccgataaggtgctgc

SEQ ID NO: 355 - DNA, 253 bp, Unknown
  16S DNA Tag
ctggccctaaacgatgaatacttggtgtctggaattattattgttccgggtgccgtcgctaacgttttaagtattccgcctggggagtacgcacgcaagtgtgaaactcaaaggaattgacggggacccgcacaagcggtggagcatgtggtttaattcgacgcaacgcgaagaaccttacctaggctagaatgtgagggaatgttgggtaacgccaacagtctgggaaaccagacccaaaacaaggtgctgc

SEQ ID NO: 356 - DNA, 249 bp, Unknown
  16S DNA Tag
cacgccataaacgatgagaactggatgtcgggagggtctgcctctcggtgtcgtagctaacgcgttaagttctccgcctggggagtacggccgcaaggttgaaactcaaaggaattgacggggacccgcacaagcggtggagcatgtggtttaattcgatgcaacgcgaagaaccttacctggtcttgacatcccaggaacctcgcagagatgtgggggtgccttcgggaacctggagacaggtgctgc

SEQ ID NO: 357 - DNA, 244 bp, Unknown
  16S DNA Tag
cacactgtaaacgatgatcactcgatgttggcgatacacagccagcgtcttagcaaaagcgataagtgatccacctggggagtacgccggcaacggtgaaactcaaaggaattgacgggggtccgcacaagcggtggagcatgtggtttaattcgatgatacgcgaggaaccttacctgggctagaatgcccttgaccgacccagagacgggtctttccgcaaggacaaggagcaaggtgctgc

SEQ ID NO: 358 - DNA, 249 bp, Unknown
  16S DNA Tag
cacgccataaacgatgaggactagacgttggaggggtaagcctttcagtgtcgtagctaacgcgctaagtcctccgcctggggagtacggccgcaaggttgaaactcaaaggaattgacggggacccgcacaagcggtggagcatgtggtttaattcgatgcaacgcgaagaaccttacctggtcttgacatccatggaaccctgcagagatgtgggggtgccgtaaggaaccatgagacaggtgctgc

SEQ ID NO: 359 - DNA, 252 bp, Unknown; identical to SEQ ID NO: 174
  16S DNA Tag

SEQ ID NO: 360 - DNA, 248 bp, Unknown
  16S DNA Tag
cacgccgtaaacggtgggcactaggtgtggggaacattccacgttctccgtgccgaagctaacgcattaagtgccccgcctggggagtacggccgcaaggctaaaactcaaaggaattgacgggggcccgcacaagcggcggagcatgcggattaattcgatgcaacgcgaagaaccttacctgggtttgacatataccggaaagctgcagagatgtagcccccgcaaggtcggtatacaggtggtgc

SEQ ID NO: 361 - DNA, 251 bp, Unknown
  16S DNA Tag
cacgctgtaaacgatgggcactaggtgtccggggtattgaccccctgggtgccgcagctaacgcattaagtgccccgcctggggagtacggtcgcaagattaaaactcaaaggaattgacgggggcccgcacaagcggtggagcatgtggttcaattcgacgcaacgcgaagaaccttacctgggctagacaacggcggaccgtcctagaaataggatcttcccttcggggactgccggttcaggtgctgc

SEQ ID NO: 362 - DNA, 248 bp, Unknown
  16S DNA Tag
ccagctgtaaacgatgcagactcggtgatgaataggcttcgtgcctattcagtgccgcagggaagccgttaagtctgccgcctggggagtacggtcgcaagactgaaacttaaaggaattggcgggggagcaccacaaggggtgaagcctgcggttcaatttggagtcaacgccggaaatcttaccgggggcgacagcagaatgaaggtcaagctgaagactttaccagacaagctgagaggaggtgc

SEQ ID NO: 363 - DNA, 251 bp, Unknown
  16S DNA Tag
cacgccgtaaacgatgggcactaggcgtcggggggagcgaccctcccggtgccgtcgctaacgcagtaagtgccccgcctggggagtacggccgcaaggttgaaactcaaaggaattgacgggggcccgcacaagcggtggagcatgtggtttaattcgaagcaacgcgaagaaccttacctgggcttgacatgcataggaaagccggtggaaacatcggcccctcttcggagcttatgcacaggtgctgc

SEQ ID NO: 364 - DNA, 253 bp, Unknown
  16S DNA Tag
ctggcagtaaacggtgtgcgtttggtgtgggaggattcgaccccttccgtgccggagctaacgcgttaaacgcaccgcctggggagtacggtcgcaagattaaaactcaaagaaattgacgggggcccgcacaagcggtggagtatgtggcttaattcgatgcaacgcgaagaaccttacctgggcttgacatgcatctctaaggcggtgaaagccgttgagtcccgcaagggacaatttgcacaggtgctgc

SEQ ID NO: 365 - DNA, 253 bp, Unknown
  16S DNA Tag
ctggcagtaaacggtgcgcgtttggtgtgggaggattcgaccccttccgtgccggagctaacgcgttaaacgcgccgcctggggagtacggtcgcaagattaaaactcaaagaaattgacgggggcccgcacaagcggtggagtatgtggcttaattcgatgcaacgcgaagaaccttacctgggcttgacatgcatctctcagtcggtgaaagccggcgagtcccgcaagggacaatttgcacaggtgctgc

SEQ ID NO: 366 - DNA, 249 bp, Unknown
  16S DNA Tag
cacgccgtaaacgttgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcattagagatagtgccccccttgtggtcggtatacaggtggtgc

SEQ ID NO: 367 - DNA, 253 bp, Unknown
  16S DNA Tag
ctggcagtaaacggtgcgcgtttggtgtgggaggattcgaccccttccgtgccggagctaacgcgttaaacgcgccgcctggggagtacggtcgcaagattaaaactcaaagaaattgacgggggcccgcacaagcggtggagtatgtggcttaattcgatgcaacgcgaagaaccttacctgggcttgacatgcatctctaagtcggtgaaagccggcgaatcccgcaagggataatttgcacaggtgctgc

SEQ ID NO: 368 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatataccggaaagcatcagagatggtgccccccttgtggtcggtatacaggtggtgc

SEQ ID NO: 369 - DNA, 255 bp, Unknown
  16S DNA Tag
ctggccgtaaacgatgggtactggatagggggctcgccgatgggctccctgtcgtagggaaaccgtgaagtaccccgcctggggagtatggtcgcaaggctgaaactcaaaggaattgacgggggctcacacaagcggtggagtatgtggcttaattcgaggctacgcgcaaaaccttatcccaggcttgacatgcacggattagccggcggaaacgtcggtgaggccgcaaggtacaacgtgcacaggtgctgc

SEQ ID NO: 370 - DNA, 250 bp, Unknown
  16S DNA Tag
cacgctgtaaacgatgtcaactagctgttggatatatgaatatatttagtggcgcagcaaacgcgataagttgaccgcctggggagtacggtcgcaagattaaaactcaaaggaattgacgggggcccgcacaagcggtggagcatgtggtttaattcgatgcaacgcgaagaaccttacctacccttgacatacagtgaactatgcagagatgcgttggtgccttcgggaacactgatacaggtgctgc

SEQ ID NO: 371 - DNA, 252 bp, Unknown
  16S DNA Tag
cacgccgtaaacgatgaatgctagacgttggcgagcatgctcgtcagtgtcgcagctaacgcattaagcattccgcctggggagtacggtcgcaagattaaaactcaaaggaattgacgggggcccgcacaagcggtggagcatgtggtttaattcgaagcaacgcgcagaaccttaccaacccttgacatggggagtatgggctggagagatccggtccttcagttcggctggctcccacacaggtgctgc

SEQ ID NO: 372 - DNA, 250 bp, Unknown
  16S DNA Tag
cacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaatttgccagagatggcttagtgctcgaaagagaaccgtaacacaggtgctgc

SEQ ID NO: 373 - DNA, 248 bp, Unknown
  16S DNA Tag
cacgccgtaaacgttgggcgctaggtgtgggggacattccacgtcctccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatatagggaaatcctccagagatggggggtccgtaagggtcctatacaggtggtgc

SEQ ID NO: 374 - DNA, 248 bp, Unknown
  16S DNA Tag
cacgccgtaaacgttgggcgctaggtgtggggctcattccacgagttccgtgccgcagctaacgcattaagcgccccgcctggggagtacggccgcaaggctaaaactcaaaggaattgacgggggcccgcacaagcggcggagcatgttgcttaattcgatgcaacgcgaagaaccttacctaggcttgacatgcacggaaatctcccagagatggggggtccgtaagggccgtgcacaggtggtgc

SEQ ID NO: 375 - DNA, 252 bp, Unknown
  16S DNA Tag
cacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgc

SEQ ID NO: 376 - DNA, 252 bp, Unknown
  16S DNA Tag
cacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 377 - DNA, 252 bp, Unknown
  16S DNA Tag
cacgccgtaaacgatgagtgctaagtgttagggggtttccgccccttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgc

SEQ ID NO: 378 - DNA, 252 bp, Unknown; identical to SEQ ID NO: 319
  16S DNA Tag

SEQ ID NO: 379 - DNA, 246 bp, Unknown
  16S DNA Tag
cacaccgtaaacgttgggcgctaggtgtgggatccattccacgggttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatatgccctgccgcctcagagatggggcttcttttgggggtgtacaggtggtgc

SEQ ID NO: 380 - DNA, 248 bp, Unknown
  16S DNA Tag
cacgctgtaaacgttgggcgctaggtgtggggggcctctccggtttcctgtgccgtagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatggccgcaaaacttccagagatggggggtccttcgggggcggtcacaggtggtgc

SEQ ID NO: 381 - DNA, 246 bp, Unknown
  16S DNA Tag
cacaccgtaaacgttgggcgctaggtgtggggtccattccacggattccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatacaccctgccgctccagagatggggcttcttttgggggtgtacaggtggtgc

SEQ ID NO: 382 - DNA, 246 bp, Unknown
  16S DNA Tag
cacaccgtaaacgttgggcgctaggtgtgggatccattccacgggttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatacaccctgccgctccagagatggggcttcttttgggggtgtacaggtggtgc

SEQ ID NO: 383 - DNA, 248 bp, Unknown
  16S DNA Tag
cacgcagtaaacgatgccgactaggcgtggggggagttgaccccctccgtgccggagccaacgcgggaagtcggccgcctggggagtacggccgcaaggctaaaactcaaaggaattgacgggggcccgcacaagcggcggagcgtgcggtttaattcgacgcgacgcgtagaaccttaccaaggcttgacatgcgcctgcatcccccggaaacggggtggccttcgagggtggcgcacaggtgctgc

SEQ ID NO: 384 - DNA, 249 bp, Unknown
  16S DNA Tag
ctagccctaaacgatgagtgcttggtgtggcgggtatcgatccctgccgtgccgaagctaacgcattaaagcactccgcctggggagtacggtcgcaaggctgaaactcaaaggaattgacgggggcccgcacaagcggtggagcatgtggtttaattcgacgcaacgcgaagaaccttacccaggctcgaacggcagatgacatccgatgaaaatcggctcccgcaagggcatctgtcgaggtgctgc

SEQ ID NO: 385 - DNA, 249 bp, Unknown
  16S DNA Tag
ccagctgtaaacgatgcagactcggtgatgggttggcttcgagccaacccagtgccgcagggaaagccgttaaagtctgccgcctggggagtacggtcgcaagactgaaacttaaaggaattggcgggggagcaccacaaggggtgaagcctgcggttcaattggagtcaacgccgggaatcttaccgggggcgacagcagagtgaaggtcaagctgaagactttaccagacaagctgagaggaggtgc

SEQ ID NO: 386 - DNA, 249 bp, Unknown
  16S DNA Tag
ccagctgtaaacgatgcagactcggtgatgagttggcttattgctaactcagtgccgcagggaaagccgttaagtttgccgcctggggagtacggtcgcaagactgaaacttaaagggaattggcgggggagcaccacaaggggtgaagcctgcggttcaattggagtcaacgccggaaatcttaccgggggcgacagcagagtgaaggtcaagctgaagactttaccagacaagctgagaggaggtgc

SEQ ID NO: 387 - DNA, 253 bp, Unknown
  16S DNA Tag
cacgctgtaaacgatgagtgctaagtgttagagggtttccgccctttagtgctgcagctaacgcattaaagcactccgcctggggagtacggccgcaaggctgaaactcaaaggaattgacggggacccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 388 - DNA, 253 bp, Unknown
  16S DNA Tag
cacgccgtaaacgatgagtgctaagtgttagagggtttccgccctttagtgctgcagcaaacgcattaaagcactccgcctggggagtacggtcgcaagactgaaactcaaaggaattgacgggggcccgcacaagcggtggagcatgtggtttaattcgaagcaacgcgaagaaccttaccaggtcttgacatcccactgcccggtatagagatatacctttcccttcggggacagtggtgacaggtggtgc

SEQ ID NO: 389 - DNA, 248 bp, Unknown
  16S DNA Tag
ccagctgtaaacgatgcagactcggtgatgggctggccttgtgccagcccagtgccgcagggaaagccgttaagtctgccgcctggggagtacggtcgcaagactgaaacttaaaggaattggcgggggagcaccacaaggggtgaagcctgcggttcaattggagtcaacgccgggaatcttaccgggggcgacagcagagtgaaggtcaagctgaagactttaccagacaagctgagaggaggtgc

SEQ ID NO: 390 - DNA, 254 bp, Unknown
  16S DNA Tag
cacgccgtaaacgatgagtgctaagtgttagagggtttccgccctttagtgctgcagctaacgcattaaagcactccgcctggggagtacggccgcaaggctgaaactcaaaggaattggacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 391 - DNA, 253 bp, Unknown
  16S DNA Tag
cacgccgtaaacgatgagtgctaagtgttagagggtttccgccctttagtgctgcagctaacgcattaaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgc

SEQ ID NO: 392 - DNA, 249 bp, Unknown
  16S DNA Tag
ctagccctaaacgatgaatgcttggtgtgacgggtatcgatccctgtcgtgccgaagctaacgcattaaagcattccgcctggggagtacggtcgcaaggctgaaactcaaaggaattgacgggggcccgcacaagcggtggagcatgtggttcaattcgacgcaacgcgaagaaccttacccaggctcgaacggcaactgacatccggcgaaagccggctcccgcaagggtagttgtcgaggtgctgc

SEQ ID NO: 393 - DNA, 255 bp, Unknown
  16S DNA Tag
cacgccgtaaacgatgagtgctaagtgttagggggtttccgccccttagtgctgcagctaacgcattaaagcactccgcctggggagtacggccgcaaggctgaaactcaaaggaattgacgggggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 394 - DNA, 253 bp, Unknown
  16S DNA Tag
cacgccgtaaacgatgagtgctaagtgttagagggtttccgccctttagtgctgcagctaacgcattaaagcactccgcctggggagtacggtcgcaagactgaaactcaaaggaattgacgggggcccgcacaagcggtggagcatgtggtttaattcgaagcaacgcgaagaaccttaccaggtcttgacatcccactgcccggtatagagatatacctttcccttcggggacagtggtgacaggtggtgc

SEQ ID NO: 395 - DNA, 253 bp, Unknown
  16S DNA Tag
cacgccgtaaacgatgagtgctaagtgttagagggtttccgccctttagtgctgcagctaacgcattaaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgtttcccttcggggaacagagtgacaggtggtgc

SEQ ID NO: 396 - DNA, 253 bp, Unknown
  16S DNA Tag
cacgccgtaaacgatgagtgctaagtgttagagggtttccgccctttagtgctgcagcaaacgcattaaagcactccgcctggggagtacggccgcaaggctgaaactcaaaggaattgacggggacccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 397 - DNA, 254 bp, Unknown
  16S DNA Tag
cacgccgtaaacgatgagtgctaagtgttagagggtttccgccctttagtgctgcagctaacgcattaaagcactccgcctggggagtacggtcgcaagactgaaactcaaaggaattggacgggggcccgcacaagcggtggagcatgtggtttaattcgaagcaacgcgaagaaccttaccaggtcttgacatcctctgacaactctagagatagagcgttccccttcgggggacagagtgacaggtggtgc

SEQ ID NO: 398 - DNA, 251 bp, Unknown
  16S DNA Tag
cacgccataaacgatgagaactggatgtcgggagggtctgcctctcggtgtcgtagctaacgcgttaaagttctccgcctggggagtacggccgcaaggttgaaactcaaaggaattgacggggacccggcacaagcggtggagcatgtggtttaattcgatgcaacgcgaagaaccttacctggtcttgacatcccaggaaccctgcagagatgtgggggtgccttcgggaacctggagacaggtgctgc

SEQ ID NO: 399 - DNA, 251 bp, Unknown
  16S DNA Tag
cacgccgtaaacgatgggcactaggtgtgggaggtgtcgactcctcccgtgccggagctaacgcattaaagtgccccgcctggggagtacggccgcaaggctaaaactcaaaggaattgacggggacccgcacaagcagcggagcatgtggtttaattcgacgcaacgcgaagaaccttacctgggtttgacatgttcctgaccgccgtggaaacacggcttccccttgtgggcaggatcacaggtggtgc

SEQ ID NO: 400 - DNA, 253 bp, Unknown
  16S DNA Tag
cacgccgtaaacgatgagtgctaagtgttagagggtttccgccctttagtgctgcagctaacgcattaaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 401 - DNA, 252 bp, Unknown
  16S DNA Tag
cacgccctaaacgatgcgaactggatgttgggtgcaactaggcactcagtatcgaagctaaacgcgttaaagttcgccgcctggggagtacggtcgcaagactgaaactcaaaggaattgacgggggcccgcacaagcggtggagtatgtggtttaattcgatgcaacgcgaagaaccttacctggccttgacatccacggaactttccagagatggattggtgccttcgggaaccgtgagacaggtgctgc

SEQ ID NO: 402 - DNA, 248 bp, Unknown
  16S DNA Tag
ccagctgtaaacgatgcagactcggtgatgaataggcttcgtgcctattcagtgccgcagggaaagccgttaagtctgccgcctggggagtacggtcgcaagactgaaacttaaaggaattggcgggggagcaccacaaggggtgaagcctgcggttcaattggagtcaacgccggaaatcttaccgggggcgacagcagaatgaaggtcaagctgaagactttaccagacaagctgagaggaggtgc

SEQ ID NO: 403 - DNA, 253 bp, Unknown
  16S DNA Tag
cacgccgtaaacgatgagtgctaagtgttagagggtttccgccctttagtgctgcagcaaacgcattaaagcactccgcctggggagtacggccgcaaggctgaaactcaaaggaattgacggggacccgcacaagcggtggagcatgtggtttaattcgaagcaacgcgaagaaccttaccaggtcttgacatctcctgacaatcctagagataggacgttccccttcgggggacaggatgacaggtggtgc

SEQ ID NO: 404 - DNA, 253 bp, Unknown
  16S DNA Tag
cacgccgtaaacgatgagtgctaagtgttagagggtttccgccctttagtgctgcagctaacgcattaaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgc

SEQ ID NO: 405 - DNA, 251 bp, Unknown
  16S DNA Tag
cacaccgtaaacgttgggcgctaggtgtgggatccattccacgggttccgtgccgcagctaacgcattaaagcgccccgcctggggagtacggccgcaaggctaaaactcaaaggaattgacgggggcccgcacaagcggcggagcatgcggattaattcgatgcaacgcgaagaaccttacctgggtttgacatatgcgagaagcctccagtgatgggggtctctttggacactcgtatacaggtggtgc

SEQ ID NO: 406 - DNA, 254 bp, Unknown
  16S DNA Tag
cacgccgtaaacgatgaatgccagccgttggcgagcttgctcgtcagtggcgcagctaaacgctttaaagcattccgcctggggagtacggtcgcaagattaaaactcaaaggaattgacgggggcccgcacaagcggtggagcatgtggtttaattcgaagcaacgcgcagaaccttaccagcctttgacatgtcccgtgtggatcctggagacaggatccttcagttcggctggcgggaacacaggtgctgc

SEQ ID NO: 407 - DNA, 251 bp, Unknown
  16S DNA Tag
cacgccgtaaacgatgagtgctaagtgttagagggtttccgccctttagtgctgcagcaaacgcattaaagcactccgcctggggagtacggtcgcaagactgaaactcaaaggaattgacggggacccgcacaagcggtggagcatgtggtttaattcgaagcaacgcgaagaaccttaccaggtcttgacatcctctgctacttctagagatagaaggttcccttcggggacagagtgacaggtggtgc

SEQ ID NO: 408 - DNA, 253 bp, Unknown
  16S DNA Tag
cacgccgtaaacgatgagtgctaagtgttagagggtttccgccctttagtgctgcagctaacgcattaaagcactccgcctggggagtacggtcgcaagactgaaactcaaaggaattgacgggggcccgcacaagcggtggagcatgtggtttaattcgaagcaacgcgaagaaccttaccaggtcttgacatcccactgaccggtatagagatatacctttcccttcggggacagtggtgacaggtggtgc

SEQ ID NO: 409 - DNA, 251 bp, Unknown
  16S DNA Tag
cacgccgtaaacgatgggcgctaggtgtgggaggtgtcgactccttccgtgccgcagctaacgcattaaagcgccccgcctggggagtacggccgcaaggctaaaactcaaaggaattgacgggggcccggcacaagcagcggagcatgtggtttaattggatgcaacgccaagaaccttaccaaggcatgacatgttcgggaaagctggagaaattcagccctccttcgggacccgatcacaggtggtgc

SEQ ID NO: 410 - DNA, 249 bp, Unknown
  16S DNA Tag
ccagctgtaaacgatgcagactcggtgatgggttggcttcgcgccaacccagtgccgcagggaaagccgttaaagtctgccgcctggggagtacggtcgcaagactgaaacttaaaggaattggcgggggagcaccacaaggggtgaagcctgcggttcaattggagtcaacgccgggaatcttaccgggggcgacagcagtgtgaaggtcaagctgaagactttaccagacaagctgagaggaggtgc

SEQ ID NO: 411 - DNA, 249 bp, Unknown
  16S DNA Tag
ctagccctaaacgatgaatgcttggtgtggcgggtatcgatccctgccgtgccgaagctaacgcattaaagcattccgcctggggagtacggtcgcaaggctgaaactcaaaggaattgacgggggcccgcacaagcggtggagcatgtggttcaattcgacgcaacgcgaagaaccttacccaggctcgaacggcattggacatccggcgaaagccggctcccgcaagggccgatgtcgaggtgctgc

SEQ ID NO: 412 - DNA, 253 bp, Unknown
  16S DNA Tag
cacgccgtaaacgatgagtgctaagtgttagagggtttccgccctttagtgctgcagctaacgcattaaagcactccgcctggggagtacggccgcaaggctgaaactcaaaggaattgacggggacccgcacaagcggtggagcatgtggtttaattcgaagcaacgcgaagaaccttaccaggtcttgacatcctctgacactcctagagataggacgttccccttcgggggacagagtgacaggtggtgc

SEQ ID NO: 413 - DNA, 253 bp, Unknown
  16S DNA Tag
cacgccgtaaacgatgagtgctaagtgttagagggtttccgccctttagtgctgcagctaacgcattaaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggactttccccttcgggggacagagtgacaggtggtgc

SEQ ID NO: 414 - DNA, 248 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacggggcccgcacaagcagcggagcatgtggcttaattcgacgcaacgcgaagaaccttaccaaggcttgacatataccggaaagcatcagagatggtgccccccttgtggtcggtatacaggtggtgc

SEQ ID NO: 415 - DNA, 248 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacggggcccgcacaagcagcggagcatgtggcttaattcgacgcaacgcgaagaaccttaccaaggcttgacatacaccggaaacggccagagatggtcgcccccttgtggtcggtgtacaggtggtgc

SEQ ID NO: 416 - DNA, 248 bp, Unknown
  16S DNA Tag
cacaccgtaaacgttgggcgctaggtgtgggacctattccatgggttccgtgccgtagctaacgcattaagcgccccgcctggggagtacggccgcaaggctaaaactcaaaggaattgacggggcccgcacaagcggcggagcatgcggattaattcgatgcaacgcgaagaaccttacctgggtttgacatacaccggaagcctctagagataggggtctctttgatactggtgtacaggtggtgc

SEQ ID NO: 417 - DNA, 248 bp, Unknown
  16S DNA Tag
cacgccttaaacgatggatactaagtgtcggcgggttaccgccggtgccgcagctaacgcagtaagtatcccgcctgggaagtacggccgcaaggttgaaactcaaaggaattgacggggcccgcacaagcggtggagcatgtggtttaattcgacgcaacgcgaagaaccttacctaggctggacatgcacgtagtaggagggtgaaagcccaacgaggtagcaataccagcgtgctcaggtgctgc

SEQ ID NO: 418 - DNA, 247 bp, Unknown
  16S DNA Tag
ctagccgtaaacgatggacactaggtgtggggggagtcgaatccctctgtgccgcagttaacgcgttaagtgtcccgcctggggagtacggccgcaaggctaaaactcaaaagaattgacggggcccgcacaagcagcggagcatgttctttaattcgatgcaacgcgaagaaccttacctggacttgacatggtgctgcaagcgcacggaaacgtgtgaccttcgagggtgcaccacagatgctgc

SEQ ID NO: 419 - DNA, 249 bp, Unknown
  16S DNA Tag
catgccgtaaacgttgggcactaggtgtgggggacattccacgttttccgcgccgtagctaacgcattaagtgccccgcctggggagtacggccgcaaggctaaaactcaaaggaattgacggggcccgcacaagcggcggagcatgcggattaattcgatgcaacgcgaagaaccttaccaaggcttgacatgggccggatcgccgcagaaatgcggtttcccttcggggccggttcacaggtggtgc

SEQ ID NO: 420 - DNA, 248 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacggggcccgcacaagcagcggagcatgtggcttaattcgacgcaacgcgaagaaccttaccaaggcttgacatacgccggaaaaccctggagacagggtcccccttgtggtcggtgtacaggtggtgc

SEQ ID NO: 421 - DNA, 248 bp, Unknown
  16S DNA Tag
cacgccgtaaacggtgggtgctaggtgtgggggacttccacgtcctctgtgccgcagctaacgcattaagcaccccgcctggggagtacggccgcaaggctaaaactcaaaggaattgacgggggcccgcacaagcggcggagcatgtggattaattcgatgcaacgcgaagaaccttacctgggcttgacatgcactggaaaccagtagagatattggcccccttgtggccggtgtacaggtggtgc

SEQ ID NO: 422 - DNA, 249 bp, Unknown
  16S DNA Tag
cacgccataaacgatgaggactagacgttggaagggtaagcctttcagtgtcgtagctaacgcgctaagtcctccgcctggggagtacggccgcaaggttgaaactcaaaggaattgacggggacccgcacaagcggtggagcatgtggtttaattcgatgcaacgcgaagaaccttacctggtcttgacatccatgggacccagcagagatgcaggggtgccgtaaggaaccatgagacaggtgctgc

SEQ ID NO: 423 - DNA, 250 bp, Unknown
  16S DNA Tag
cacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgtctggaattttgcagagatgcgaaagtgcccgaaagggagccagaacacaggtgctgc

SEQ ID NO: 424 - DNA, 247 bp, Unknown
  16S DNA Tag
cacgctgtaaacgttgggcgctaggtgtggggaccttccacggtttccgcgccgtagctaacgcattaagcgccccgcctggggagtacggccgcaaggctaaaactcaaaggaattgacgggggcccgcacaagcggcggagcatgttgcttaattcgacgcaacgcgaagaaccttaccaaggcttgacattgcccggaaacactcagagatgggtgcctcttcggatcgggtgacaggtggtgc

SEQ ID NO: 425 - DNA, 249 bp, Unknown
  16S DNA Tag
cacaccgtaaacgttgggcgctaggtgtgggatccattccacgggttccgtgccgcagctaacgcattaagcgccccgcctggggagtacggccgcaaggctaaaactcaaaggaattgacgggggcccgcacaagcggcggagcatgcggattaattcgatgcaacgcgaagaaccttacctgggtttgacatacgccggaagcctccagagatgggggtctctttgatactggtgtacaggtggtgc

SEQ ID NO: 426 - DNA, 248 bp, Unknown; identical to SEQ ID NO: 51
  16S DNA Tag

SEQ ID NO: 427 - DNA, 250 bp, Unknown; identical to SEQ ID NO: 81
  16S DNA Tag

SEQ ID NO: 428 - DNA, 252 bp, Unknown; identical to SEQ ID NO: 11
  16S DNA Tag

SEQ ID NO: 429 - DNA, 251 bp, Unknown
  16S DNA Tag
ctagccgtaaacgatggacactaggtgttggtggtatcaaccccgccagtgccgaagctaacgcattaagtgtcccgcctggggagtacggccgcaaggctaaaactcaaaggaattgacgggggcccgcacaagcagcggagcgtggggtttaattcgacgcaacgcgaagaaccttaccaaggcttgacatgaccaggacagcggcggaaacgtcgtcttctcgcaagggacctggccacaggtgttgc

SEQ ID NO: 430 - DNA, 252 bp, Unknown
  16S DNA Tag
cacgccgtaaacgatgaatgctagccgttggcgagcttgctcgtcagtggcgcagctaacgctttaagcattccgcctggggagtacggtcgcaagattaaaactcaaaggaattgacgggggcccgcacaagcggtggagcatgtggttcaattcgaagcaacgcgcagaaccttaccagcccttgacatcccggtcgcggacgccagagacggtgtccttcagttcggctggaccggagacaggtgctgc

SEQ ID NO: 431 - DNA, 252 bp, Unknown
  16S DNA Tag
cacgccgtaaacgatgaatgctagccgttggcgagcttgctcgtcagtggcgcagctaacgctttaagcattccgcctggggagtacggtcgcaagattaaaactcaaaggaattgacgggggcccgcacaagcggtggagcatgtggttcaattcgaagcaacgcgcagaaccttaccagcccttgacatcccggtcgcggtttccagagacggagaccttcagttcggctggaccggagacaggtgctgc

SEQ ID NO: 432 - DNA, 252 bp, Unknown; identical to SEQ ID NO: 58
  16S DNA Tag

SEQ ID NO: 433 - DNA, 250 bp, Unknown; identical to SEQ ID NO: 84
  16S DNA Tag

SEQ ID NO: 434 - DNA, 250 bp, Unknown
  16S DNA Tag
cacgccgtaaacgatgaatgccagccgttagtgggtttactcactagtggcgcagctaacgctttaagcattccgcctggggagtacggtcgcaagattaaaactcaaaggaattgacgggggcccgcacaagcggtggagcatgtggtttaattcgacgcaacgcgcagaaccttaccagcccttgacatgtccaggaccggtcgcagagatgtgaccttctcttcggagcctggagcacaggtgctgc

SEQ ID NO: 435 - DNA, 250 bp, Unknown; identical to SEQ ID NO: 152
  16S DNA Tag

SEQ ID NO: 436 - DNA, 249 bp, Unknown
  16S DNA Tag
catgccctaaacggtgcacactaggtcttggaggattcgaccccttcagggcccaagctaacgcgttaagtgtgccgcctgaggactacggtcgcaagactaaaactcaaaggaattgacgggggcccgcacaagcggtggagtatgtggcttaattcgatgcaacgcgaagaaccttacctagccttgacatgcactagaccgagcctgaaagggctctttccttcgggactggtgcacaggtgctgc

SEQ ID NO: 437 - DNA, 248 bp, Unknown
  16S DNA Tag
cacgccgtaaacgttgggcgctaggtgtcggggaccttccacgtcctcggtgccgcagctaacgcattaagcgccccgcctggggagtacggccgcaaggctaaaactcaaaggaattgacgggggcccgcacaagcggcggagcatgtggcttaattcgacgcaacgcgaagaaccttacctgggtttgacatgcagggaaatctcatagagatatgaggtccgcaagggtcctgcacaggtggtgc

SEQ ID NO: 438 - DNA, 247 bp, Unknown
  16S DNA Tag
cacgccctaaacgatgattactcgacatacgcgatacactgtgtgtgtctgagcgaaagcattaagtaatccacctgggaagtacgttcgcaagaatgaaactcaaaggaattgacgggggtccgcacaagcggtggagcatgtggtttaattcgatgatacgcgaggaaccttacctgggctagaatgcagtctgaccgccggtgaaagctggttttgtagcaatacacagattgtaaggtgctgc

SEQ ID NO: 439 - DNA, 247 bp, Unknown
  16S DNA Tag
cacgccctaaacgatgattactcgacatcagcgatacactgttggtgtctgagcgaaagcattaagtaatccacctgggaagtacgttcgcaagaatgaaactcaaaggaattgacgggggtccgcacaagcggtggagcatgtggtttaattcgatgatacgcgaggaaccttacctgggctagaatgcagtctgaccgtgggtgaaagctcatcttgtagcaatacacagattgtaaggtgctgc

SEQ ID NO: 440 - DNA, 248 bp, Unknown
  16S DNA Tag
cacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcctgtgttacccgtagagatatggggtccacttcggtggcgcagagacaggtgctgc

SEQ ID NO: 441 - DNA, 250 bp, Unknown
  16S DNA Tag
cacgccgtaaacgatggatactagctgttggaggtatcgaccccttcagtagcgaagctaacgcgttaagtatcccgcctgtggagtacggtcgcaagactaaaacataaaggaattgacggggacccgcacaagcggtggatcgtgttctttaattcgatgctaaacgaagaaccttaccagggcttgacatctagggaatttttgggaaaccaattagtgccttttggaaccctatgacaggtgatgc

SEQ ID NO: 442 - DNA, 253 bp, Unknown
  16S DNA Tag
ctagctgtaaacggtgcatatttgctgtaaaaggaatcgaccccttttgtggcgtagcaaacgcgttaaatatgccgcctgggaagtacggtcgcaagattaaaactcaaagaaattgacgggggcctgcacaagcggtggagtatgtggcttaattcgatgcaacgcgaagaaccttacctggccttgacatgcatatagtagaagggtgaaagcctgacgaggtagcaataccagtatgcacaggtgctgc

SEQ ID NO: 443 - DNA, 249 bp, Unknown; identical to SEQ ID NO: 38
  16S DNA Tag

SEQ ID NO: 444 - DNA, 250 bp, Unknown
  16S DNA Tag
cacgccgtaaactatggatgctagctgttataggtatcgacccctgtagtagcgaagctaacgcgttaagcatcccgcctgtggagtacggtcgcaagactaaaacataaaggaattgacggggacccgcacaagcggtggagcgtgttgtttaattcgatggtaagcgaagaaccttacccaggtttgacatcctcggaagcactgcgaaagcagagtgtgccttttggaaccgagtgacaggtgctgc

SEQ ID NO: 445 - DNA, 253 bp, Unknown
  16S DNA Tag
cacgccgtaaacgatgataactagctgtccgggcacatggtgtttgggtggcgcagctaacgcattaagttatccgcctggggagtacggtcgcaagattaaaactcaaaggaattgacgggggcctgcacaagcggtggagcatgtggtttaattcgaagcaacgcgcagaaccttaccagcgtttgacatcctcatcgcggataccagagatggtttccttcagttcggctggatgagtgacaggtgctgc

SEQ ID NO: 446 - DNA, 249 bp, Unknown
  16S DNA Tag
cacgccataaacgatgagaactagacgttgaaggggtaagccctccagtgtcgtagctaacgcgctaagttctccgcctggggagtacggccgcaaggttgaaactcaaaggaattgacggggacccgcacaagcggtggagcatgtggtttaattcgatgcaacgcgaagaaccttacctggtcttgacatcctgggaatcccgtagagatatgggagtgccgcaaggaacccagagacaggtgctgc

SEQ ID NO: 447 - DNA, 247 bp, Unknown
  16S DNA Tag
cacgccctaaacgatgattactcgacatacgcgatacactgtgtgtgtctgagcgaaagcattaagtaatccacctgggaagtacgaccgcaaggttgaaactcaaaggaattggcgggggtccgcacaagcggtggagcatgtggtttaattcgatgatacgcgaggaaccttacctgggctagaatgcggtctgaccgccggtgaaagctggttttgtagcaatacacagatcgtaaggtgctgc

SEQ ID NO: 448 - DNA, 249 bp, Unknown
  16S DNA Tag
cacgccgtaaacgatgtctactagccgttggggtccttgaggccttagtggcgcagctaacgcactaagtagaccgcctggggagtacggtcgcaagattaaaactcaaatgaattgacgggggcccgcacaagcggtggagcatgtggtttaattcgacgcaacgcgaagaaccttaccaggtcttgacatccagagaactttccagagatggattggtgccttcgggaactctgagacaggtgctgc

SEQ ID NO: 449 - DNA, 249 bp, Unknown
  16S DNA Tag
catgccgtaaacgttgggcgctaggtgtggggactttccacggtttccgcgccgtagctaacgcattaagcgccccgcctggggagtacggccgcaaggctaaaactcaaaggaattgacgggggcccgcacaagcggcggagcatgttgcttaattcgacgcaacgcgaagaaccttaccaaggtttgacatcacccgtggactcgcagagatgtgaggtcatttagttggcgggtgacaggtggtgc

SEQ ID NO: 450 - DNA, 247 bp, Unknown
  16S DNA Tag
cacgccctaaacgatggatactcgacatacgcgatacactgtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagtctgaccgtgggtgaaagctcattttgtagcaatacacagattgtaaggtgctgc

SEQ ID NO: 451 - DNA, 250 bp, Unknown
  16S DNA Tag
cacgccgtaaacgatggatactagctgttggaggtatcgaccccttcagtagcgaagctaacgcgttaagtatcccgcctgtggagtacggtcgcaagactaaaacataaaggaattgacggggacccgcacaagcggtggatcgtgttctttaattcgatgataaacgaagaaccttaccagggcttgacatccagggaagcaccacgaaagtggagtgtgccttttggaaccctgtgacaggtgatgc

SEQ ID NO: 452 - DNA, 250 bp, Unknown
  16S DNA Tag
cacgccctaaacgatgcgaactggatgttgggttcaactaggaactcagtatcgaagctaacgcgttaagttcgccgcctggggagtacggtcgcaagactgaaactcaaaggaattgacgggggcccgcacaagcggtggagtatgtggtttaattcgatgcaacgcgcagaaccttacctggccttgacatccacggaactttccagagatggattggtgccttcgggaaccgtgagacaggtgctgc

SEQ ID NO: 453 - DNA, 250 bp, Unknown
  16S DNA Tag
catgccgtaaacgttgggcgctagatgtggggaccattccacggtttccgtgtcgcagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatttacgagaacgctgcagaaatgcagaactctttggacactcgtaaacaggtggtgc

SEQ ID NO: 454 - DNA, 249 bp, Unknown
  16S DNA Tag
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacgtccagagatgggcgcccccttgtggtcggtgtacaggtggtgc

SEQ ID NO: 455 - DNA, 249 bp, Unknown
  16S DNA Tag
cacgccgtaaacggtgggcactaggtgtgggcgacattccacgtcgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaacgtccagagatgggcgcccccttgtggtcggtgtacaggtggtgc

SEQ ID NO: 456 - DNA, 247 bp, Unknown
  16S DNA Tag
cacgctgtaaacgttgggcgctaggtgtgggggtcttccacgatctccgtgccggagctaacgcattaagcgccccgcctggggagtacggccgcaaggctaaaactcaaaggaattgacgggggcccgcacaagcggcggagcatgttgcttaattcgacgcaacgcgaagaaccttaccaaggtttgacatcacccggacagctgcagagatgtggccttcttcggactgggtgacaggtggtgc

SEQ ID NO: 457 - DNA, 247 bp, Unknown
  16S DNA Tag
cacgccgtaaacggtgggtgctaggtgtgggggacttccacgtcctccgtgccgcagctaacgcattaagcaccccgcctggggagtacggccgcaaggctaaaactcaaaggaattgacgggggcccgcacaagcggcggagcatgtggattaattcgatgcaacgcgaagaaccttacctgggcttgacatgcaccggaaacctgcagagatgtaggcctcttcggactggtgtacaggtggtgc

SEQ ID NO: 458 - DNA, 251 bp, Unknown
  16S DNA Tag
ctagccctaaacgatggatgcttggtgtgttgggtacccaatcccaacgtgccgaagctaacgcgataagcatcccgcctggggagtacggtcgcaaggctgaaactcaaaggaattgacgggggcccgcacaagcggtggagcatgtggttcaattcgacgcaacgcgaagaaccttacctgggctcgaagcgcagtggaccggggtagaaatatccctttctcgcaagagactgctgcggaggtgctgc

SEQ ID NO: 459 - DNA, 248 bp, Unknown
  16S DNA Tag
ctagccctaaacgatgaatgcttggtgtggcgggtatcgatccctgccgtgccgaagctaacgcattaagcattccgcctggggagtacggtcgcaaggctgaaactcaaaggaattgacgggggcccgcacaagcggtggagcatgtggttcaattcgacgcaacgcgaagaaccttacccaggctcgaacggcattggacatccggcgaaagccggctcccgcaagggccgatgtcgaggtgctgc

SEQ ID NO: 460 - DNA, 249 bp, Unknown
  16S DNA Tag
ctagccctaaacgatggatgtttggtgtgacaggtacccaatcctgtcgtgccggagctaacgcgttaaacatcccgcctggggagtacggtcgcaaggctgaaactcaaaggaattgacgggggcccgcacaagcggtggagcatgtggttcaattcgacgcaacgcgaagaaccttacctgggctcgaaatgtagtggatcggggtagaaatatcctttcccgcaagggctgctatataggtgctgc

SEQ ID NO: 461 - DNA, 267 bp, Unknown
  16S DNA Tag
ctagccctaaacgttgggtactgggtagtagactagacatgggtttactgccgcagcaaaagtgctaagtaccccgcctggggagtatggtcgcaaggctgaaactcaaaggaattgacgggggctcacacaagcggtggagcatgtggcttaattcgaggctacgcgaagaaccttatcctggacttgacatgtgcgaaagcgtcaggtggtagaagccggaaacggtaacgaatcccgcaagggagagcctgatacaggtgctgc

SEQ ID NO: 462 - DNA, 1448 bp, Arthrobacter globiformis
gatgaacgctggcggcgtgcttaacacatgcaagtcgaacgatgatccggtgcttgcaccggggattagtggcgaacgggtgagtaacacgtgagtaacctgcccttgactctgggataagcctgggaaactgggtctaataccggatatgactcctcatcgcatggtggggggtggaaagcttttgtggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgccgtgaaagtccggggctcaactccggatctgcggtgggtacgggcaggctagagtgatgtaggggagactggaattcctggtgtagcggtgaaatgcgcagatatcaggaggaacaccgatggcgaaggcaggtctctgggcattaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaccgccgcagagatgtggtttctccttttggggccggttcacaggtggtgcatggttgtcgtcagctcgtgtcgtgagatgttgggttaagtcccgcaacgagcgcaaccctcgttccatgttgccagcgcgtaatggcggggactcatgggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaaccccttgtgggagggagccgtcgaaggtgggactggcgattgggact

SEQ ID NO: 463 - DNA, 1344 bp, Streptomyces vastus
gacgaacgctggcggcgtgcttaacacatgcaagtcgaacgatgaaccacttcggtggggattagtggcgaacgggtgagtaacacgtgggcaatctgccctgcactctgggacaagccctggaaacggggtctaataccggataatacttccactctcctgggtgggggttgaaagctccggcggtgcaggatgagcccgcggcctatcagctagtcggtgaggtaatggctcaccgaggcgacgacgggtagccggcctgagagggcgaccggccacactgggactgagacacggcccagactcctacgggaggcagcagtggggaatattgcacaatgggcgcaagcctgatgcagcgacgccgcgtgggggatgacggccttcgggttgtaaacctctttcagcagggaagaagcgcaagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagctcggggcttaaccccgggtctgcattcgatacgggctggctggagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcagcggagcatgtggcttaattcgacgcaacgcgaagaaccttaccaaggcttgacatacgccggaaaaccctggagacagggtcccccttgtggtcggtgtacaggtggtgcatggctgtcgtcagctcgtgtcgtgagatgttgggttaagtcccgcaacgagcgcaacccttgtcccgtgttgccagcaacggtttcggccggttggggactcacgggagaccgccggggtcaactcggaggaaggtggggacgacgtcaagtcatcatgccccttatgtcttgggctgcacacgtgctacaatggccggtacaatgagctgcgataccgcaaggtggagcgaatctcaaaaagccggtctcagttcggattggggtctgcaactcgaccccatgaagtcggagttgctagtaatcgcagatcagcagtgctgcggtgaatacgttcccgggcct

SEQ ID NO: 464 - DNA, 1344 bp, Streptomyces vastus
gacgaacgctggcggcgtgcttaacacatgcaagtcgaacgatgaaccacttcggtggggattagtggcgaacgggtgagtaacacgtgggcaatctgccctgcactctgggacaagccctggaaacggggtctaataccggataatacttccactctcctgggtgggggttgaaagctccggcggtgcaggatgagcccgcggcctatcagctagtcggtgaggtaatggctcaccgaggcgacgacgggtagccggcctgagagggcgaccggccacactgggactgagacacggcccagactcctacgggaggcagcagtggggaatattgcacaatgggcgcaagcctgatgcagcgacgccgcgtgggggatgacggccttcgggttgtaaacctctttcagcagggaagaagcgtaagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagctcggggcttaaccccgggtctgcattcgatacgggctggctggagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcagcggagcatgtggcttaattcgacgcaacgcgaagaaccttaccaaggcttgacatacgccggaaaaccctggagacagggtcccccttgtggtcggtgtacaggtggtgcatggctgtcgtcagctcgtgtcgtgagatgttgggttaagtcccgcaacgagcgcaacccttgtcccgtgttgccagcaacggtttcggccggttggggactcacgggagaccgccggggtcaactcggaggaaggtggggacgacgtcaagtcatcatgccccttatgtcttgggctgcacacgtgctacaatggccggtacaatgagctgcgataccgcaaggtggagcgaatctcaaaaagccggtctcagttcggattggggtctgcaactcgaccccatgaagtcggagttgctagtaatcgcagatcagcagtgctgcggtgaatacgttcccgggcct

SEQ ID NO: 465 - DNA, 1451 bp, Niastella yeongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcactggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttggggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggtcaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaatgatacggtgaatacgttcccggaccttgcacacaccgcccgtcaagccatggaagctgggtgtacctaaagtcggtaaccgcaaggagccgcctagggtaaaactagtgactggggct

SEQ ID NO: 466 - DNA, 1451 bp, Niastella yeongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcaccggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttggggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggtcaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaatgatacggtgaatacgttcccggaccttgcacacaccgcccgtcaagccatggaagctgggtgtacctaaagtcggtaaccgcaaggagccgcctagggtaaaactagtgactggggct

SEQ ID NO: 467 - DNA, 1451 bp, Niastella yeongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcaccggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttgaggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggttaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaacgatacggtgaatacgttcccggaccttgcacacaccgcccgtcaagccatggaagctgggtgtacctaaagtcggtaaccgcaaggagccgcctagggtaaaactagtgactggggct

SEQ ID NO: 468 - DNA, 247 bp, Niastella yeongjuensis; identical to SEQ ID NO: 294

SEQ ID NO: 469 - DNA, 249 bp, Streptomyces roseiscleroticus; identical to SEQ ID NO: 183

SEQ ID NO: 470 - DNA, 249 bp, Streptomyces galilaeus
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaagcatcagagatggtgccccccttgtggtcggtgtacaggtggtgc

SEQ ID NO: 471 - DNA, 252 bp, Bacillus megaterium; identical to SEQ ID NO: 162

SEQ ID NO: 472 - DNA, 252 bp, Bacillus marisflavi; identical to SEQ ID NO: 315

SEQ ID NO: 473 - DNA, 252 bp, Bacillus simplex; identical to SEQ ID NO: 97

SEQ ID NO: 474 - DNA, 252 bp, Bacillus kribbensis; identical to SEQ ID NO: 307

SEQ ID NO: 475 - DNA, 857 bp, Zea mays
ccccatcaccattgctgcgacgagagtgagcgggagagggtaggtggcgaggcggcggagatggggcgggggccggtgcagctgcgccggatcgagaacaagatcaaccgccaggtgaccttctccaagcgccggaacgggctgctgaagaaggcccacgagatctccgtgctctgcgacgcagaggtcgcgctcatcgtcttctccactaaggggaagctctacgagtactctagccattccagcatggaaggcattcttgagcgttaccagcgttactcatttgaagaaagggcagtacttaacccaagtattgaagaccaggcaaattggggagatgaatatgtccggttaaaatccaaacttgatgcacttcagaagagtcaaaggcagctgttaggagaacaattgagttcactgaccataaaagaactccagcaactggagcaacaactggacagttctttgaagcatattaggtcaagaaagaatcagctcatgttcgattcaatttccgcgcttcagaaaaaggagaaagcacttacagatcaaaacggtgtcctgcaaaagttcatggaggcagagaaggagaaaaacaaggctttgatgaacgcgcagctccgggagcagcaaaatggagcatcaacaagctccccatcactttcaccaccaatagttccagattccatgccaactctaaatatagggccatgtcaacatagaggggcagcagaatctgagtctgaaccgtctcctgctcctgcacaagcaaacaggggcaacctgccaccatggatgctccgcactgtcaagtaacaggtgaggtcttcccagtgtagttttgcagctgatctcga

SEQ ID NO: 476 - protein, 251 aa, Zea mays
MGRGPVQLRRIENKINRQVTFSKRRNGLLKKAHEISVLCDAEVALIVFSTKGKLYEYSSHSSMEGILERYQRYSFEERAVLNPSIEDQANWGDEYVRLKSKLDALQKSQRQLLGEQLSSLTIKELQQLEQQLDSSLKHIRSRKNQLMFDSISALQKKEKALTDQNGVLQKFMEAEKEKNKALMNAQLREQQNGASTSSPSLSPPIVPDSMPTLNIGPCQHRGAAESESEPSPAPAQANRGNLPPWMLRTVK